SEQUENCE LISTING (55 sequences)

SEQ ID NO: 1 - DNA, 1866 bp, adeno-associated virus 2
  REP78 Start Codon (1)..(3)
  REP52 Start Codon (673)..(675)
atgccggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 2 - DNA, 1872 bp, Artificial Sequence
  AAV2 REP78 optimized for Nicotiana benthamiana
  REP78 Start Codon (7)..(9)
  REP52 Start Codon (679)..(681)
gggtttatgactggtttctacgaaatcgttattaaggttccatctgatttggatggtcatcttcctggaatctctgattcattcgttaactgggttgctgaaaaagagtgggaattgccacctgattcagatcttgatttgaatcttatcgaacaagctccacttactgttgctgagaagttgcaaagagattttcttacagagtggagaagggtttctaaggctcctgaggctcttttctttgttcaattcgaaaagggagagtcatacttccatttgcatgttcttgttgaaactacaggtgttaagtcattggttcttggaagatttttgtctcaaatcagagaaaagcttatccaaagaatctataggggtattgagccaactttgcctaattggtttgctgttactaagacaagaaatggtgctggaggtggaaataaggttgttgatgaatgttacatcccaaactaccttttgccaaagactcaacctgaacttcaatgggcttggacaaatttggagcaatatctttctgcttgtttgaatcttacagagagaaaaaggttggttgctcaacatcttactcatgtttctcaaacacaagaacaaaataaggagaaccaaaacccaaactcagatgctcctgttattagatcaaaaacttctgctaggtacatggaattggttggttggcttgttgataagggaattacatctgaaaaacagtggattcaagaggatcaagcttcatacatctcttttaatgctgcttctaactcaagatctcaaattaaggctgctcttgataatgctggaaagattatgtcattgactaaaacagctccagattatcttgttggacaacaacctgttgaagatatctcttcaaacagaatctataagatcttggagcttaatggttacgatccacaatacgctgcttctgtttttcttggttgggctactaagaaattcggaaagaggaacacaatttggctttttggtcctgctactacaggaaaaactaatattgctgaagctattgctcatacagttccattctacggttgtgttaactggactaatgagaacttcccttttaatgattgtgttgataagatggttatttggtgggaagagggaaagatgacagctaaagttgttgaatcagctaaggctattttgggtggatctaaagttagagttgatcaaaagtgtaaatcttcagctcaaattgatccaactcctgttattgttacttcaaacacaaacatgtgtgctgttattgatggtaactctactacattcgaacatcaacaacctcttcaagataggatgttcaagttcgagttgactagaaggcttgatcatgattttggaaaggttacaaagcaagaggttaaggatttctttagatgggctaaagatcatgttgttgaggttgaacatgagttttacgttaagaaaggtggagctaagaaaaggccagctccttcagatgctgatatttctgaaccaaagagagttagggagtcagttgctcaaccttcaacatctgatgctgaagcttctattaattacgctgatagataccaaaataagtgttcaaggcatgttggtatgaatttgatgctttttccatgtagacaatgtgagaggatgaatcaaaactctaacatctgtttcactcatggacaaaaggattgtttggaatgtttcccagtttcagagtctcaacctgtttcagttgttaagaaagcttaccaaaagctttgttacatccatcatatcatgggaaaagttcctgatgcttgtacagcttgtgatttggttaatgttgatcttgatgattgtatttttgaacaataa

SEQ ID NO: 3 - DNA, 1872 bp, Artificial Sequence
  AAV2 REP78 optimized for Arabidopsis thaliana
  REP78 Start Codon (7)..(9)
  REP52 Start Codon (679)..(681)
gggtttatgactggtttttatgaaattgttattaaggttccttctgatcttgatggacatcttcctggaatttctgattcttttgttaattgggttgctgaaaaggaatgggaacttcctcctgattctgatctggatcttaatcttattgaacaagctcctcttactgttgctgaaaagcttcaaagagattttcttactgaatggagaagagtttctaaggctcctgaagctcttttttttgttcaatttgaaaagggagaatcttattttcatttgcatgttcttgttgaaactactggagttaagtctttggttcttggaagatttctttctcaaattagagaaaagcttattcaaagaatttatagaggaattgaacctactcttcctaattggtttgctgttactaagactagaaatggagctggaggaggaaataaggttgttgatgaatgttatattcctaattatcttcttcctaagactcaacctgaacttcaatgggcttggactaatttggaacaatatctttctgcttgtcttaatcttactgaaagaaagagacttgttgctcaacatcttactcatgtttctcaaactcaagaacaaaataaggaaaatcaaaatcctaattctgatgctcctgttattagatctaagacttctgctagatatatggaacttgttggatggcttgttgataagggaattacttctgaaaagcaatggattcaagaagatcaagcttcttatatttcttttaatgctgcttctaattctagatctcaaattaaggctgctcttgataatgctggaaagattatgtctcttactaagactgctcctgattatcttgttggacaacaacctgttgaagatatttcttctaatagaatttataagattcttgaacttaatggatatgatcctcaatatgctgcttctgtttttcttggatgggctactaagaagtttggaaagagaaatactatttggctttttggacctgctactactggaaagactaatattgctgaagctattgctcatactgttcctttttatggatgtgttaattggactaatgaaaattttccttttaatgattgtgttgataagatggttatttggtgggaagaaggaaagatgactgctaaggttgttgaatctgctaaggctattcttggaggatctaaggttagagttgatcaaaagtgtaagtcttctgctcaaattgatcctactcctgttattgttacttctaatactaatatgtgtgctgttattgatggaaattctactacttttgaacatcaacaacctcttcaagatagaatgtttaagtttgaacttactagaagacttgatcatgattttggaaaggttactaagcaagaagttaaggatttttttagatgggctaaggatcatgttgttgaagttgaacatgaattttatgttaagaagggaggagctaagaagagacctgctccttctgatgctgatatttctgaacctaagagagttagagaatctgttgctcaaccttctacttctgatgctgaagcttctattaattatgctgatagatatcaaaataagtgttctagacatgttggaatgaatcttatgctttttccttgtagacaatgtgaaagaatgaatcaaaattctaatatttgttttactcatggacaaaaggattgtcttgaatgttttcctgtttctgaatctcaacctgtttctgttgttaagaaggcttatcaaaagctttgttatattcatcatattatgggaaaggttcctgatgcttgtactgcttgtgatcttgttaatgttgatcttgatgattgtatttttgaacaatga

SEQ ID NO: 4 - DNA, 1872 bp, Artificial Sequence
  AAV2 REP78 optimized for Solanum tuberosum
  REP78 Start Codon (7)..(9)
  REP52 Start Codon (679)..(681)
gggtttatgactggtttttatgaaattgttattaaggttccttctgatcttgatggacatcttcctggaatttctgattcttttgttaattgggttgctgaaaaggaatgggaacttcctcctgattctgatctggatcttaatcttattgaacaagctcctcttactgttgctgaaaagcttcaaagagattttcttactgaatggagaagagtttctaaggctcctgaagctcttttttttgttcaatttgaaaagggagaatcttattttcatttgcatgttcttgttgaaactactggagttaagtctttggttcttggaagatttctttctcaaattagagaaaagcttattcaaagaatttatagaggaattgaacctactcttcctaattggtttgctgttactaagactagaaatggagctggaggaggaaataaggttgttgatgaatgttatattcctaattatcttcttcctaagactcaacctgaacttcaatgggcttggactaatttggaacaatatctttctgcttgtcttaatcttactgaaagaaagagacttgttgctcaacatcttactcatgtttctcaaactcaagaacaaaataaggaaaatcaaaatcctaattctgatgctcctgttattagatctaagacttctgctagatatatggaacttgttggatggcttgttgataagggaattacttctgaaaagcaatggattcaagaagatcaagcttcttatatttcttttaatgctgcttctaattctagatctcaaattaaggctgctcttgataatgctggaaagattatgtctcttactaagactgctcctgattatcttgttggacaacaacctgttgaagatatttcttctaatagaatttataagattcttgaacttaatggatatgatcctcaatatgctgcttctgtttttcttggatgggctactaagaagtttggaaagagaaatactatttggctttttggacctgctactactggaaagactaatattgctgaagctattgctcatactgttcctttttatggatgtgttaattggactaatgaaaattttccttttaatgattgtgttgataagatggttatttggtgggaagaaggaaagatgactgctaaggttgttgaatctgctaaggctattcttggaggatctaaggttagagttgatcaaaagtgtaagtcttctgctcaaattgatcctactcctgttattgttacttctaatactaatatgtgtgctgttattgatggaaattctactacttttgaacatcaacaacctcttcaagatagaatgtttaagtttgaacttactagaagacttgatcatgattttggaaaggttactaagcaagaagttaaggatttttttagatgggctaaggatcatgttgttgaagttgaacatgaattttatgttaagaagggaggagctaagaagagacctgctccttctgatgctgatatttctgaacctaagagagttagagaatctgttgctcaaccttctacttctgatgctgaagcttctattaattatgctgatagatatcaaaataagtgttctagacatgttggaatgaatcttatgctttttccttgtagacaatgtgaaagaatgaatcaaaattctaatatttgttttactcatggacaaaaggattgtcttgaatgttttcctgtttctgaatctcaacctgtttctgttgttaagaaggcttatcaaaagctttgttatattcatcatattatgggaaaggttcctgatgcttgtactgcttgtgatcttgttaatgttgatcttgatgattgtatttttgaacaataa

SEQ ID NO: 5 - DNA, 1872 bp, Artificial Sequence
  AAV2 REP78 optimized for Cannabis sativa
  REP78 Start Codon (7)..(9)
  REP52 Start Codon (679)..(681)
gggtttatgactggtttttatgaaattgttattaaagttccttcagatttggatggacatttgcctggaatttcagattcatttgttaattgggttgctgaaaaagaatgggaattgcctcctgattcagatctggatttgaatttgattgaacaagctcctttgactgttgctgaaaaattgcaaagagattttttgactgaatggagaagagtttcaaaagctcctgaagctttgttttttgttcaatttgaaaaaggagaatcatattttcatttgcatgttttggttgaaactactggagttaaatcattggttttgggaagatttttgtcacaaattagagaaaaattgattcaaagaatttatagaggaattgaacctactttgcctaattggtttgctgttactaaaactagaaatggagctggaggaggaaataaagttgttgatgaatgctatattcctaattatttgttgcctaaaactcaacctgaattgcaatgggcttggactaatttggaacaatatttgtcagcttgcttgaatttgactgaaagaaaaagattggttgctcaacatttgactcatgtttcacaaactcaagaacaaaataaagaaaatcaaaatcctaattcagatgctcctgttattagatcaaaaacttcagctagatatatggaattggttggatggttggttgataaaggaattacttcagaaaaacaatggattcaagaagatcaagcttcatatatttcatttaatgctgcttcaaattcaagatcacaaattaaagctgctttggataatgctggaaaaattatgtcattgactaaaactgctcctgattatttggttggacaacaacctgttgaagatatttcatcaaatagaatttataaaattttggaattgaatggatatgatcctcaatatgctgcttcagtttttttgggatgggctactaaaaaatttggaaaaagaaatactatttggttgtttggacctgctactactggaaaaactaatattgctgaagctattgctcatactgttcctttttatggatgcgttaattggactaatgaaaattttccttttaatgattgcgttgataaaatggttatttggtgggaagaaggaaaaatgactgctaaagttgttgaatcagctaaagctattttgggaggatcaaaagttagagttgatcaaaaatgcaaatcatcagctcaaattgatcctactcctgttattgttacttcaaatactaatatgtgcgctgttattgatggaaattcaactacttttgaacatcaacaacctttgcaagatagaatgtttaaatttgaattgactagaagattggatcatgattttggaaaagttactaaacaagaagttaaagatttttttagatgggctaaagatcatgttgttgaagttgaacatgaattttatgttaaaaaaggaggagctaaaaaaagacctgctccttcagatgctgatatttcagaacctaaaagagttagagaatcagttgctcaaccttcaacttcagatgctgaagcttcaattaattatgctgatagatatcaaaataaatgctcaagacatgttggaatgaatttgatgttgtttccttgcagacaatgcgaaagaatgaatcaaaattcaaatatttgctttactcatggacaaaaagattgcttggaatgctttcctgtttcagaatcacaacctgtttcagttgttaaaaaagcttatcaaaaattgtgctatattcatcatattatgggaaaagttcctgatgcttgcactgcttgcgatttggttaatgttgatttggatgattgcatttttgaacaataa

SEQ ID NO: 6 - DNA, 1872 bp, Artificial Sequence
  AAV2 REP78 optimized for Fagopyrum esculentum
  REP78 Start Codon (7)..(9)
  REP52 Start Codon (679)..(681)
gggtttatgactggtttctacgagatcgttatcaaggttccttccgatctcgatggacatctccctggaatctccgattccttcgttaactgggttgctgagaaggagtgggagctccctcctgattccgatctggatctcaacctcatcgagcaggctcctctcaccgttgctgagaagctccagagggatttcctcaccgagtggaggagggtttccaaggctcctgaggctctcttcttcgttcagttcgagaagggagagtcctacttccatttgcatgttctcgttgagaccaccggagttaagtccttggttctcggaaggttcctctcccagatcagggagaagctcatccagaggatctacaggggaatcgagcctaccctccctaactggttcgctgttaccaagaccaggaacggagctggaggaggaaacaaggttgttgatgagtgctacatccctaactacctcctccctaagacccagcctgagctccagtgggcttggaccaacttggagcagtacctctccgcttgcctcaacctcaccgagaggaagaggctcgttgctcagcatctcacccatgtttcccagacccaggagcagaacaaggagaaccagaaccctaactccgatgctcctgttatcaggtccaagacctccgctaggtacatggagctcgttggatggctcgttgataagggaatcacctccgagaagcagtggatccaggaggatcaggcttcctacatctccttcaacgctgcttccaactccaggtcccagatcaaggctgctctcgataacgctggaaagatcatgtccctcaccaagaccgctcctgattacctcgttggacagcagcctgttgaggatatctcctccaacaggatctacaagatcctcgagctcaacggatacgatcctcagtacgctgcttccgttttcctcggatgggctaccaagaagttcggaaagaggaacaccatctggctcttcggacctgctaccaccggaaagaccaacatcgctgaggctatcgctcataccgttcctttctacggatgcgttaactggaccaacgagaacttccctttcaacgattgcgttgataagatggttatctggtgggaggagggaaagatgaccgctaaggttgttgagtccgctaaggctatcctcggaggatccaaggttagggttgatcagaagtgcaagtcctccgctcagatcgatcctacccctgttatcgttacctccaacaccaacatgtgcgctgttatcgatggaaactccaccaccttcgagcatcagcagcctctccaggataggatgttcaagttcgagctcaccaggaggctcgatcatgatttcggaaaggttaccaagcaggaggttaaggatttcttcaggtgggctaaggatcatgttgttgaggttgagcatgagttctacgttaagaagggaggagctaagaagaggcctgctccttccgatgctgatatctccgagcctaagagggttagggagtccgttgctcagccttccacctccgatgctgaggcttccatcaactacgctgataggtaccagaacaagtgctccaggcatgttggaatgaacctcatgctcttcccttgcaggcagtgcgagaggatgaaccagaactccaacatctgcttcacccatggacagaaggattgcctcgagtgcttccctgtttccgagtcccagcctgtttccgttgttaagaaggcttaccagaagctctgctacatccatcatatcatgggaaaggttcctgatgcttgcaccgcttgcgatctcgttaacgttgatctcgatgattgcatcttcgagcagtaa

SEQ ID NO: 7 - DNA, 1872 bp, Artificial Sequence
  AAV2 REP78 optimized for Oryza sativa
  REP78 Start Codon (7)..(9)
  REP52 Start Codon (679)..(681)
gggtttatgactggtttctacgagatcgtgatcaaggtgccgtccgacctcgacggccacctcccgggcatctccgactccttcgtgaactgggtggccgagaaggagtgggagctcccgccggactccgacctggacctcaacctcatcgagcaggccccgctcaccgtggccgagaagctccagagggacttcctcaccgagtggaggagggtgtccaaggccccggaggccctcttcttcgtgcagttcgagaagggcgagtcctacttccacttgcacgtgctcgtggagaccaccggcgtgaagtccttggtgctcggcaggttcctctcccagatcagggagaagctcatccagaggatctacaggggcatcgagccgaccctcccgaactggttcgccgtgaccaagaccaggaacggcgccggcggcggcaacaaggtggtggacgagtgctacatcccgaactacctcctcccgaagacccagccggagctccagtgggcctggaccaacttggagcagtacctctccgcctgcctcaacctcaccgagaggaagaggctcgtggcccagcacctcacccacgtgtcccagacccaggagcagaacaaggagaaccagaacccgaactccgacgccccggtgatcaggtccaagacctccgccaggtacatggagctcgtgggctggctcgtggacaagggcatcacctccgagaagcagtggatccaggaggaccaggcctcctacatctccttcaacgccgcctccaactccaggtcccagatcaaggccgccctcgacaacgccggcaagatcatgtccctcaccaagaccgccccggactacctcgtgggccagcagccggtggaggacatctcctccaacaggatctacaagatcctcgagctcaacggctacgacccgcagtacgccgcctccgtgttcctcggctgggccaccaagaagttcggcaagaggaacaccatctggctcttcggcccggccaccaccggcaagaccaacatcgccgaggccatcgcccacaccgtgccgttctacggctgcgtgaactggaccaacgagaacttcccgttcaacgactgcgtggacaagatggtgatctggtgggaggagggcaagatgaccgccaaggtggtggagtccgccaaggccatcctcggcggctccaaggtgagggtggaccagaagtgcaagtcctccgcccagatcgacccgaccccggtgatcgtgacctccaacaccaacatgtgcgccgtgatcgacggcaactccaccaccttcgagcaccagcagccgctccaggacaggatgttcaagttcgagctcaccaggaggctcgaccacgacttcggcaaggtgaccaagcaggaggtgaaggacttcttcaggtgggccaaggaccacgtggtggaggtggagcacgagttctacgtgaagaagggcggcgccaagaagaggccggccccgtccgacgccgacatctccgagccgaagagggtgagggagtccgtggcccagccgtccacctccgacgccgaggcctccatcaactacgccgacaggtaccagaacaagtgctccaggcacgtgggcatgaacctcatgctcttcccgtgcaggcagtgcgagaggatgaaccagaactccaacatctgcttcacccacggccagaaggactgcctcgagtgcttcccggtgtccgagtcccagccggtgtccgtggtgaagaaggcctaccagaagctctgctacatccaccacatcatgggcaaggtgccggacgcctgcaccgcctgcgacctcgtgaacgtggacctcgacgactgcatcttcgagcagtga

SEQ ID NO: 8 - DNA, 1872 bp, Artificial Sequence
  AAV2 REP78 optimized for Zea mays
  REP78 Start Codon (7)..(9)
  REP52 Start Codon (679)..(681)
gggtttatgactggtttctacgagatcgtgatcaaggtgccgtccgacctggacggccacctgccgggcatctccgactccttcgtgaactgggtggccgagaaggagtgggagctgccgccggactccgacctggacctgaacctgatcgagcaggccccgctgaccgtggccgagaagctgcagagggacttcctgaccgagtggaggagggtgtccaaggccccggaggccctgttcttcgtgcagttcgagaagggcgagtcctacttccacttgcacgtgctggtggagaccaccggcgtgaagtccttggtgctgggcaggttcctgtcccagatcagggagaagctgatccagaggatctacaggggcatcgagccgaccctgccgaactggttcgccgtgaccaagaccaggaacggcgccggcggcggcaacaaggtggtggacgagtgctacatcccgaactacctgctgccgaagacccagccggagctgcagtgggcctggaccaacttggagcagtacctgtccgcctgcctgaacctgaccgagaggaagaggctggtggcccagcacctgacccacgtgtcccagacccaggagcagaacaaggagaaccagaacccgaactccgacgccccggtgatcaggtccaagacctccgccaggtacatggagctggtgggctggctggtggacaagggcatcacctccgagaagcagtggatccaggaggaccaggcctcctacatctccttcaacgccgcctccaactccaggtcccagatcaaggccgccctggacaacgccggcaagatcatgtccctgaccaagaccgccccggactacctggtgggccagcagccggtggaggacatctcctccaacaggatctacaagatcctggagctgaacggctacgacccgcagtacgccgcctccgtgttcctgggctgggccaccaagaagttcggcaagaggaacaccatctggctgttcggcccggccaccaccggcaagaccaacatcgccgaggccatcgcccacaccgtgccgttctacggctgcgtgaactggaccaacgagaacttcccgttcaacgactgcgtggacaagatggtgatctggtgggaggagggcaagatgaccgccaaggtggtggagtccgccaaggccatcctgggcggctccaaggtgagggtggaccagaagtgcaagtcctccgcccagatcgacccgaccccggtgatcgtgacctccaacaccaacatgtgcgccgtgatcgacggcaactccaccaccttcgagcaccagcagccgctgcaggacaggatgttcaagttcgagctgaccaggaggctggaccacgacttcggcaaggtgaccaagcaggaggtgaaggacttcttcaggtgggccaaggaccacgtggtggaggtggagcacgagttctacgtgaagaagggcggcgccaagaagaggccggccccgtccgacgccgacatctccgagccgaagagggtgagggagtccgtggcccagccgtccacctccgacgccgaggcctccatcaactacgccgacaggtaccagaacaagtgctccaggcacgtgggcatgaacctgatgctgttcccgtgcaggcagtgcgagaggatgaaccagaactccaacatctgcttcacccacggccagaaggactgcctggagtgcttcccggtgtccgagtcccagccggtgtccgtggtgaagaaggcctaccagaagctgtgctacatccaccacatcatgggcaaggtgccggacgcctgcaccgcctgcgacctggtgaacgtggacctggacgactgcatcttcgagcagtga

SEQ ID NO: 9 - DNA, 1872 bp, Artificial Sequence
  AAV2 REP78 optimized for Solanum lycopersicoides
  REP78 Start Codon (7)..(9)
  REP52 Start Codon (679)..(681)
gggtttatgactggtttttacgagattgttattaaggttccatcagatcttgatggacatcttccaggaatttcagattcatttgttaattgggttgcagagaaggagtgggagcttccaccagattcagatctggatcttaatcttattgagcaagcaccacttacagttgcagagaagcttcaaagagattttcttacagagtggagaagagtttcaaaggcaccagaggcacttttttttgttcaatttgagaagggagagtcatactttcatttgcatgttcttgttgagacaacaggagttaagtcattggttcttggaagatttctttcacaaattagagagaagcttattcaaagaatttacagaggaattgagccaacacttccaaattggtttgcagttacaaagacaagaaatggagcaggaggaggaaataaggttgttgatgagtgttacattccaaattaccttcttccaaagacacaaccagagcttcaatgggcatggacaaatttggagcaatacctttcagcatgtcttaatcttacagagagaaagagacttgttgcacaacatcttacacatgtttcacaaacacaagagcaaaataaggagaatcaaaatccaaattcagatgcaccagttattagatcaaagacatcagcaagatacatggagcttgttggatggcttgttgataagggaattacatcagagaagcaatggattcaagaggatcaagcatcatacatttcatttaatgcagcatcaaattcaagatcacaaattaaggcagcacttgataatgcaggaaagattatgtcacttacaaagacagcaccagattaccttgttggacaacaaccagttgaggatatttcatcaaatagaatttacaagattcttgagcttaatggatacgatccacaatacgcagcatcagtttttcttggatgggcaacaaagaagtttggaaagagaaatacaatttggctttttggaccagcaacaacaggaaagacaaatattgcagaggcaattgcacatacagttccattttacggatgtgttaattggacaaatgagaattttccatttaatgattgtgttgataagatggttatttggtgggaggagggaaagatgacagcaaaggttgttgagtcagcaaaggcaattcttggaggatcaaaggttagagttgatcaaaagtgtaagtcatcagcacaaattgatccaacaccagttattgttacatcaaatacaaatatgtgtgcagttattgatggaaattcaacaacatttgagcatcaacaaccacttcaagatagaatgtttaagtttgagcttacaagaagacttgatcatgattttggaaaggttacaaagcaagaggttaaggatttttttagatgggcaaaggatcatgttgttgaggttgagcatgagttttacgttaagaagggaggagcaaagaagagaccagcaccatcagatgcagatatttcagagccaaagagagttagagagtcagttgcacaaccatcaacatcagatgcagaggcatcaattaattacgcagatagataccaaaataagtgttcaagacatgttggaatgaatcttatgctttttccatgtagacaatgtgagagaatgaatcaaaattcaaatatttgttttacacatggacaaaaggattgtcttgagtgttttccagtttcagagtcacaaccagtttcagttgttaagaaggcataccaaaagctttgttacattcatcatattatgggaaaggttccagatgcatgtacagcatgtgatcttgttaatgttgatcttgatgattgtatttttgagcaatga

SEQ ID NO: 10 - DNA, 1872 bp, Artificial Sequence
  AAV2 REP78 optimized for Solanum lycopersicum
  REP78 Start Codon (7)..(9)
  REP52 Start Codon (679)..(681)
gggtttatgactggtttttatgaaattgttattaaggttccttctgatcttgatggacatcttcctggaatttctgattcttttgttaattgggttgctgaaaaggaatgggaacttcctcctgattctgatcttgatcttaatcttattgaacaagctcctcttactgttgctgaaaagcttcaaagagattttcttactgaatggagaagagtttctaaggctcctgaagctcttttttttgttcaatttgaaaagggagaatcttattttcatcttcatgttcttgttgaaactactggagttaagtctcttgttcttggaagatttctttctcaaattagagaaaagcttattcaaagaatttatagaggaattgaacctactcttcctaattggtttgctgttactaagactagaaatggagctggaggaggaaataaggttgttgatgaatgttatattcctaattatcttcttcctaagactcaacctgaacttcaatgggcttggactaatcttgaacaatatctttctgcttgtcttaatcttactgaaagaaagagacttgttgctcaacatcttactcatgtttctcaaactcaagaacaaaataaggaaaatcaaaatcctaattctgatgctcctgttattagatctaagacttctgctagatatatggaacttgttggatggcttgttgataagggaattacttctgaaaagcaatggattcaagaagatcaagcttcttatatttcttttaatgctgcttctaattctagatctcaaattaaggctgctcttgataatgctggaaagattatgtctcttactaagactgctcctgattatcttgttggacaacaacctgttgaagatatttcttctaatagaatttataagattcttgaacttaatggatatgatcctcaatatgctgcttctgtttttcttggatgggctactaagaagtttggaaagagaaatactatttggctttttggacctgctactactggaaagactaatattgctgaagctattgctcatactgttcctttttatggatgtgttaattggactaatgaaaattttccttttaatgattgtgttgataagatggttatttggtgggaagaaggaaagatgactgctaaggttgttgaatctgctaaggctattcttggaggatctaaggttagagttgatcaaaagtgtaagtcttctgctcaaattgatcctactcctgttattgttacttctaatactaatatgtgtgctgttattgatggaaattctactacttttgaacatcaacaacctcttcaagatagaatgtttaagtttgaacttactagaagacttgatcatgattttggaaaggttactaagcaagaagttaaggatttttttagatgggctaaggatcatgttgttgaagttgaacatgaattttatgttaagaagggaggagctaagaagagacctgctccttctgatgctgatatttctgaacctaagagagttagagaatctgttgctcaaccttctacttctgatgctgaagcttctattaattatgctgatagatatcaaaataagtgttctagacatgttggaatgaatcttatgctttttccttgtagacaatgtgaaagaatgaatcaaaattctaatatttgttttactcatggacaaaaggattgtcttgaatgttttcctgtttctgaatctcaacctgtttctgttgttaagaaggcttatcaaaagctttgttatattcatcatattatgggaaaggttcctgatgcttgtactgcttgtgatcttgttaatgttgatcttgatgattgtatttttgaacaataa

SEQ ID NO: 11 - DNA, 1872 bp, Artificial Sequence
  AAV2 REP78 optimized for Lactuca sativa
  REP78 Start Codon (7)..(9)
  REP52 Start Codon (679)..(681)
gggtttatgactggtttttatgaaattgttattaaagttccatctgatcttgatggacatcttccaggaatttctgattcttttgttaattgggttgctgaaaaagaatgggaacttccaccagattctgatcttgatcttaatcttattgaacaagctccacttacagttgctgaaaaacttcaaagagattttcttacagaatggagaagagtttctaaagctccagaagctcttttttttgttcaatttgaaaaaggagaatcttattttcatcttcatgttcttgttgaaacaacaggagttaaatctcttgttcttggaagatttctttctcaaattagagaaaaacttattcaaagaatttatagaggaattgaaccaacacttccaaattggtttgctgttacaaaaacaagaaatggagctggaggaggaaataaagttgttgatgaatgttatattccaaattatcttcttccaaaaacacaaccagaacttcaatgggcttggacaaatcttgaacaatatctttctgcttgtcttaatcttacagaaagaaaaagacttgttgctcaacatcttacacatgtttctcaaacacaagaacaaaataaagaaaatcaaaatccaaattctgatgctccagttattagatctaaaacatctgctagatatatggaacttgttggatggcttgttgataaaggaattacatctgaaaaacaatggattcaagaagatcaagcttcttatatttcttttaatgctgcttctaattctagatctcaaattaaagctgctcttgataatgctggaaaaattatgtctcttacaaaaacagctccagattatcttgttggacaacaaccagttgaagatatttcttctaatagaatttataaaattcttgaacttaatggatatgatccacaatatgctgcttctgtttttcttggatgggctacaaaaaaatttggaaaaagaaatacaatttggctttttggaccagctacaacaggaaaaacaaatattgctgaagctattgctcatacagttccattttatggatgtgttaattggacaaatgaaaattttccatttaatgattgtgttgataaaatggttatttggtgggaagaaggaaaaatgacagctaaagttgttgaatctgctaaagctattcttggaggatctaaagttagagttgatcaaaaatgtaaatcttctgctcaaattgatccaacaccagttattgttacatctaatacaaatatgtgtgctgttattgatggaaattctacaacatttgaacatcaacaaccacttcaagatagaatgtttaaatttgaacttacaagaagacttgatcatgattttggaaaagttacaaaacaagaagttaaagatttttttagatgggctaaagatcatgttgttgaagttgaacatgaattttatgttaaaaaaggaggagctaaaaaaagaccagctccatctgatgctgatatttctgaaccaaaaagagttagagaatctgttgctcaaccatctacatctgatgctgaagcttctattaattatgctgatagatatcaaaataaatgttctagacatgttggaatgaatcttatgctttttccatgtagacaatgtgaaagaatgaatcaaaattctaatatttgttttacacatggacaaaaagattgtcttgaatgttttccagtttctgaatctcaaccagtttctgttgttaaaaaagcttatcaaaaactttgttatattcatcatattatgggaaaagttccagatgcttgtacagcttgtgatcttgttaatgttgatcttgatgattgtatttttgaacaatga

SEQ ID NO: 12 - protein, 621 aa, adeno-associated virus 2
MPGFYEIVIKVPSDLDGHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 13 - protein, 621 aa, Artificial Sequence
  AAV2 REP78 optimized for plant expression
MTGFYEIVIKVPSDLDGHLPGISDSFVNWVAEKEWELPPDSDLDLNLIEQAPLTVAEKLQRDFLTEWRRVSKAPEALFFVQFEKGESYFHLHVLVETTGVKSLVLGRFLSQIREKLIQRIYRGIEPTLPNWFAVTKTRNGAGGGNKVVDECYIPNYLLPKTQPELQWAWTNLEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 14 - DNA, 2208 bp, adeno-associated virus 2
  VP1 Start Codon (1)..(3)
  VP2 Start Codon (412)..(414)
  VP3 Start Codon (607)..(609)
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 15 - DNA, 2214 bp, Artificial Sequence
  AAV2 CAP optimized for Nicotiana benthamiana
  VP1 Start Codon (7)..(9)
  VP2 Start Codon (418)..(420)
  VP3 Start Codon (613)..(615)
gggtttatgactgccgccggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctactggatcaggtgctcctatggctgataataacgaaggtgctgatggagttggtaattcatctggaaattggcattgtgattctacttggatgggagatagagttattactacatcaactaggacatgggctcttccaacatacaataaccatttgtacaagcaaatttcatctcaatcaggagcttctaacgataaccattacttcggatactctacaccatggggttacttcgatttcaacagattccattgtcatttttcacctagagattggcaaaggcttattaataacaattggggttttagaccaaagaggcttaacttcaagttgtttaatatccaagttaaagaagttactcaaaacgatggaactacaactatcgctaataaccttacttctacagttcaagtttttacagattcagagtatcaacttccttacgttttgggatctgctcatcaaggttgtttgccaccttttccagctgatgtttttatggttcctcaatatggttaccttactttgaataacggatctcaagctgttggtagatcatctttctactgtcttgaatacttcccttctcaaatgttgaggacaggaaataacttcactttttcatacacattcgaggatgttccatttcattcatcttacgctcattcacaatctcttgatagattgatgaatcctcttatcgatcaatatctttactacttgtctagaactaacacaccatcaggtacaactacacaatcaaggcttcaattttctcaagctggagcttcagatattagagatcaatctaggaattggttgccaggtccttgttacagacaacaaagggtttcaaagacttctgctgataataacaattcagaatactcttggactggagctacaaaataccatcttaatggtagggattctttggttaatccaggacctgctatggcttcacataaggatgatgaagagaagtttttcccacaatctggagttcttatcttcggaaagcaaggttcagaaaagactaacgttgatatcgagaaggttatgatcacagatgaagaggaaatcagaactacaaatcctgttgctactgagcaatacggttcagtttctacaaatttgcaaagaggaaataggcaagctgctactgctgatgttaatacacaaggagttcttcctggtatggtttggcaagatagggatgtttacttgcaaggtccaatttgggctaaaattcctcatactgatggacattttcatccatctcctcttatgggaggttttggtttgaagcatccacctccacaaatccttattaaaaacacaccagttcctgctaatccttcaactacattttctgctgctaagttcgcttcttttattactcaatactctacaggacaagtttcagttgagattgaatgggagttgcaaaaggaaaactcaaaaagatggaacccagagatccaatacacttctaactacaataagtcagttaacgttgatttcactgttgatacaaatggtgtttactctgaaccaaggcctattggaactagataccttacaaggaatttgtaa

SEQ ID NO: 16 - DNA, 2214 bp, Artificial Sequence
  AAV2 CAP optimized for Arabidopsis thaliana
  VP1 Start Codon (7)..(9)
  VP2 Start Codon (418)..(420)
  VP3 Start Codon (613)..(615)
gggtttatgactgccgccggttatcttcctgattggcttgaagatactctttctgaaggaattagacaatggtggaagcttaagcctggacctcctcctcctaagcctgctgaaagacataaggatgattctagaggacttgttcttcctggatataagtatcttggaccttttaatggacttgataagggagaacctgttaatgaagctgatgctgctgctcttgaacatgataaggcttatgatagacaacttgattctggagataatccttatcttaagtataatcatgctgatgctgaatttcaagaaagacttaaggaagatacttcttttggaggaaatcttggaagagctgtttttcaagctaagaagagagttcttgaacctcttggacttgttgaagaacctgttaagacggctcctggaaagaagagacctgttgaacattctcctgttgaacctgattcttcttctggaactggaaaggctggacaacaacctgctagaaagagacttaattttggacaaactggagatgctgattctgttcctgatcctcaacctcttggacaacctcctgctgctccttctggacttggaactaatactatggctactggatctggagctcctatggctgataataatgaaggagctgatggagttggaaattcttctggaaattggcattgtgattctacttggatgggagatagagttattactacttctactagaacttgggctcttcctacttataataatcatctttataagcaaatttcttctcaatctggagcttctaatgataatcattattttggatattctactccttggggatattttgattttaatagatttcattgtcatttttctcctagagattggcaaagacttattaataataattggggatttagacctaagagacttaattttaagctttttaatattcaagttaaggaagttactcaaaatgatggaactactactattgctaataatcttacttctactgttcaagtttttactgattctgaatatcaacttccttatgttcttggatctgctcatcaaggatgtcttcctccttttcctgctgatgtttttatggttcctcaatatggatatcttactcttaataatggatctcaagctgttggaagatcttctttttattgtcttgaatattttccttctcaaatgcttagaactggaaataattttactttttcttatacttttgaagatgttccttttcattcttcttatgctcattctcaatctcttgatagacttatgaatcctcttattgatcaatatctttattatctttctagaactaatactccttctggaactactactcaatctagacttcaattttctcaagctggagcttctgatattagagatcaatctagaaattggcttcctggaccttgttatagacaacaaagagtttctaagacttctgctgataataataattctgaatattcttggactggagctactaagtatcatcttaatggaagagattctcttgttaatcctggacctgctatggcttctcataaggatgatgaagaaaagttttttcctcaatctggagttcttatttttggaaagcaaggatctgaaaagactaatgttgatattgaaaaggttatgattactgatgaagaagaaattagaactactaatcctgttgctactgaacaatatggatctgtttctactaatcttcaaagaggaaatagacaagctgctactgctgatgttaatactcaaggagttcttcctggaatggtttggcaagatagagatgtttatcttcaaggacctatttgggctaagattcctcatactgatggacattttcatccttctcctcttatgggaggatttggacttaagcatcctcctcctcaaattcttattaagaatactcctgttcctgctaatccttctactactttttctgctgctaagtttgcttcttttattactcaatattctactggacaagtttctgttgaaattgaatgggaacttcaaaaggaaaattctaagagatggaatcctgaaattcaatatacttctaattataataagtctgttaatgttgattttactgttgatactaatggagtttattctgaacctagacctattggaactagatatcttactagaaatctttga

SEQ ID NO: 17 - DNA, 2214 bp, Artificial Sequence
  AAV2 CAP optimized for Solanum tuberosum
  VP1 Start Codon (7)..(9)
  VP2 Start Codon (418)..(420)
  VP3 Start Codon (613)..(615)
gggtttatgactgccgccggttatcttcctgattggcttgaagatactctttctgaaggaattagacaatggtggaagcttaagcctggacctcctcctcctaagcctgctgaaagacataaggatgattctagaggacttgttcttcctggatataagtatcttggaccttttaatggacttgataagggagaacctgttaatgaagctgatgctgctgctcttgaacatgataaggcttatgatagacaacttgattctggagataatccttatcttaagtataatcatgctgatgctgaatttcaagaaagacttaaggaagatacttcttttggaggaaatcttggaagagctgtttttcaagctaagaagagagttcttgaacctcttggacttgttgaagaacctgttaagacggctcctggaaagaagagacctgttgaacattctcctgttgaacctgattcttcttctggaactggaaaggctggacaacaacctgctagaaagagacttaattttggacaaactggagatgctgattctgttcctgatcctcaacctcttggacaacctcctgctgctccttctggacttggaactaatactatggctactggatctggagctcctatggctgataataatgaaggagctgatggagttggaaattcttctggaaattggcattgtgattctacttggatgggagatagagttattactacttctactagaacttgggctcttcctacttataataatcatctttataagcaaatttcttctcaatctggagcttctaatgataatcattattttggatattctactccttggggatattttgattttaatagatttcattgtcatttttctcctagagattggcaaagacttattaataataattggggatttagacctaagagacttaattttaagctttttaatattcaagttaaggaagttactcaaaatgatggaactactactattgctaataatcttacttctactgttcaagtttttactgattctgaatatcaacttccttatgttcttggatctgctcatcaaggatgtcttcctccttttcctgctgatgtttttatggttcctcaatatggatatcttactcttaataatggatctcaagctgttggaagatcttctttttattgtcttgaatattttccttctcaaatgcttagaactggaaataattttactttttcttatacttttgaagatgttccttttcattcttcttatgctcattctcaatctcttgatagacttatgaatcctcttattgatcaatatctttattatctttctagaactaatactccttctggaactactactcaatctagacttcaattttctcaagctggagcttctgatattagagatcaatctagaaattggcttcctggaccttgttatagacaacaaagagtttctaagacttctgctgataataataattctgaatattcttggactggagctactaagtatcatcttaatggaagagattctcttgttaatcctggacctgctatggcttctcataaggatgatgaagaaaagttttttcctcaatctggagttcttatttttggaaagcaaggatctgaaaagactaatgttgatattgaaaaggttatgattactgatgaagaagaaattagaactactaatcctgttgctactgaacaatatggatctgtttctactaatcttcaaagaggaaatagacaagctgctactgctgatgttaatactcaaggagttcttcctggaatggtttggcaagatagagatgtttatcttcaaggacctatttgggctaagattcctcatactgatggacattttcatccttctcctcttatgggaggatttggacttaagcatcctcctcctcaaattcttattaagaatactcctgttcctgctaatccttctactactttttctgctgctaagtttgcttcttttattactcaatattctactggacaagtttctgttgaaattgaatgggaacttcaaaaggaaaattctaagagatggaatcctgaaattcaatatacttctaattataataagtctgttaatgttgattttactgttgatactaatggagtttattctgaacctagacctattggaactagatatcttactagaaatctttaa

SEQ ID NO: 18 - DNA, 2214 bp, Artificial Sequence
  AAV2 CAP optimized for Cannabis sativa
  VP1 Start Codon (7)..(9)
  VP2 Start Codon (418)..(420)
  VP3 Start Codon (613)..(615)
gggtttatgactgccgccggttatttgcctgattggttggaagatactttgtcagaaggaattagacaatggtggaaattgaaacctggacctcctcctcctaaacctgctgaaagacataaagatgattcaagaggattggttttgcctggatataaatatttgggaccttttaatggattggataaaggagaacctgttaatgaagctgatgctgctgctttggaacatgataaagcttatgatagacaattggattcaggagataatccttatttgaaatataatcatgctgatgctgaatttcaagaaagattgaaagaagatacttcatttggaggaaatttgggaagagctgtttttcaagctaaaaaaagagttttggaacctttgggattggttgaagaacctgttaaaacggctcctggaaaaaaaagacctgttgaacattcacctgttgaacctgattcatcatcaggaactggaaaagctggacaacaacctgctagaaaaagattgaattttggacaaactggagatgctgattcagttcctgatcctcaacctttgggacaacctcctgctgctccttcaggattgggaactaatactatggctactggatcaggagctcctatggctgataataatgaaggagctgatggagttggaaattcatcaggaaattggcattgcgattcaacttggatgggagatagagttattactacttcaactagaacttgggctttgcctacttataataatcatttgtataaacaaatttcatcacaatcaggagcttcaaatgataatcattattttggatattcaactccttggggatattttgattttaatagatttcattgccatttttcacctagagattggcaaagattgattaataataattggggatttagacctaaaagattgaattttaaattgtttaatattcaagttaaagaagttactcaaaatgatggaactactactattgctaataatttgacttcaactgttcaagtttttactgattcagaatatcaattgccttatgttttgggatcagctcatcaaggatgcttgcctccttttcctgctgatgtttttatggttcctcaatatggatatttgactttgaataatggatcacaagctgttggaagatcatcattttattgcttggaatattttccttcacaaatgttgagaactggaaataattttactttttcatatacttttgaagatgttccttttcattcatcatatgctcattcacaatcattggatagattgatgaatcctttgattgatcaatatttgtattatttgtcaagaactaatactccttcaggaactactactcaatcaagattgcaattttcacaagctggagcttcagatattagagatcaatcaagaaattggttgcctggaccttgctatagacaacaaagagtttcaaaaacttcagctgataataataattcagaatattcatggactggagctactaaatatcatttgaatggaagagattcattggttaatcctggacctgctatggcttcacataaagatgatgaagaaaaattttttcctcaatcaggagttttgatttttggaaaacaaggatcagaaaaaactaatgttgatattgaaaaagttatgattactgatgaagaagaaattagaactactaatcctgttgctactgaacaatatggatcagtttcaactaatttgcaaagaggaaatagacaagctgctactgctgatgttaatactcaaggagttttgcctggaatggtttggcaagatagagatgtttatttgcaaggacctatttgggctaaaattcctcatactgatggacattttcatccttcacctttgatgggaggatttggattgaaacatcctcctcctcaaattttgattaaaaatactcctgttcctgctaatccttcaactactttttcagctgctaaatttgcttcatttattactcaatattcaactggacaagtttcagttgaaattgaatgggaattgcaaaaagaaaattcaaaaagatggaatcctgaaattcaatatacttcaaattataataaatcagttaatgttgattttactgttgatactaatggagtttattcagaacctagacctattggaactagatatttgactagaaatttgtaa

SEQ ID NO: 19 - DNA, 2214 bp, Artificial Sequence
  AAV2 CAP optimized for Fagopyrum esculentum
  VP1 Start Codon (7)..(9)
  VP2 Start Codon (418)..(420)
  VP3 Start Codon (613)..(615)
gggtttatgactgccgccggttatctccctgattggctcgaggataccctctccgagggaatcaggcagtggtggaagctcaagcctggacctcctcctcctaagcctgctgagaggcataaggatgattccaggggactcgttctccctggatacaagtacctcggacctttcaacggactcgataagggagagcctgttaacgaggctgatgctgctgctctcgagcatgataaggcttacgataggcagctcgattccggagataacccttacctcaagtacaaccatgctgatgctgagttccaggagaggctcaaggaggatacctccttcggaggaaacctcggaagggctgttttccaggctaagaagagggttctcgagcctctcggactcgttgaggagcctgttaagacggctcctggaaagaagaggcctgttgagcattcccctgttgagcctgattcctcctccggaaccggaaaggctggacagcagcctgctaggaagaggctcaacttcggacagaccggagatgctgattccgttcctgatcctcagcctctcggacagcctcctgctgctccttccggactcggaaccaacaccatggctaccggatccggagctcctatggctgataacaacgagggagctgatggagttggaaactcctccggaaactggcattgcgattccacctggatgggagatagggttatcaccacctccaccaggacctgggctctccctacctacaacaaccatctctacaagcagatctcctcccagtccggagcttccaacgataaccattacttcggatactccaccccttggggatacttcgatttcaacaggttccattgccatttctcccctagggattggcagaggctcatcaacaacaactggggattcaggcctaagaggctcaacttcaagctcttcaacatccaggttaaggaggttacccagaacgatggaaccaccaccatcgctaacaacctcacctccaccgttcaggttttcaccgattccgagtaccagctcccttacgttctcggatccgctcatcagggatgcctccctcctttccctgctgatgttttcatggttcctcagtacggatacctcaccctcaacaacggatcccaggctgttggaaggtcctccttctactgcctcgagtacttcccttcccagatgctcaggaccggaaacaacttcaccttctcctacaccttcgaggatgttcctttccattcctcctacgctcattcccagtccctcgataggctcatgaaccctctcatcgatcagtacctctactacctctccaggaccaacaccccttccggaaccaccacccagtccaggctccagttctcccaggctggagcttccgatatcagggatcagtccaggaactggctccctggaccttgctacaggcagcagagggtttccaagacctccgctgataacaacaactccgagtactcctggaccggagctaccaagtaccatctcaacggaagggattccctcgttaaccctggacctgctatggcttcccataaggatgatgaggagaagttcttccctcagtccggagttctcatcttcggaaagcagggatccgagaagaccaacgttgatatcgagaaggttatgatcaccgatgaggaggagatcaggaccaccaaccctgttgctaccgagcagtacggatccgtttccaccaacctccagaggggaaacaggcaggctgctaccgctgatgttaacacccagggagttctccctggaatggtttggcaggatagggatgtttacctccagggacctatctgggctaagatccctcataccgatggacatttccatccttcccctctcatgggaggattcggactcaagcatcctcctcctcagatcctcatcaagaacacccctgttcctgctaacccttccaccaccttctccgctgctaagttcgcttccttcatcacccagtactccaccggacaggtttccgttgagatcgagtgggagctccagaaggagaactccaagaggtggaaccctgagatccagtacacctccaactacaacaagtccgttaacgttgatttcaccgttgataccaacggagtttactccgagcctaggcctatcggaaccaggtacctcaccaggaacctctaa

SEQ ID NO: 20 - DNA, 2214 bp, Artificial Sequence
  AAV2 CAP optimized for Oryza sativa
  VP1 Start Codon (7)..(9)
  VP2 Start Codon (418)..(420)
  VP3 Start Codon (613)..(615)
gggtttatgactgccgccggttatctcccggactggctcgaggacaccctctccgagggcatcaggcagtggtggaagctcaagccgggcccgccgccgccgaagccggccgagaggcacaaggacgactccaggggcctcgtgctcccgggctacaagtacctcggcccgttcaacggcctcgacaagggcgagccggtgaacgaggccgacgccgccgccctcgagcacgacaaggcctacgacaggcagctcgactccggcgacaacccgtacctcaagtacaaccacgccgacgccgagttccaggagaggctcaaggaggacacctccttcggcggcaacctcggcagggccgtgttccaggccaagaagagggtgctcgagccgctcggcctcgtggaggagccggtgaagacggccccgggcaagaagaggccggtggagcactccccggtggagccggactcctcctccggcaccggcaaggccggccagcagccggccaggaagaggctcaacttcggccagaccggcgacgccgactccgtgccggacccgcagccgctcggccagccgccggccgccccgtccggcctcggcaccaacaccatggccaccggctccggcgccccgatggccgacaacaacgagggcgccgacggcgtgggcaactcctccggcaactggcactgcgactccacctggatgggcgacagggtgatcaccacctccaccaggacctgggccctcccgacctacaacaaccacctctacaagcagatctcctcccagtccggcgcctccaacgacaaccactacttcggctactccaccccgtggggctacttcgacttcaacaggttccactgccacttctccccgagggactggcagaggctcatcaacaacaactggggcttcaggccgaagaggctcaacttcaagctcttcaacatccaggtgaaggaggtgacccagaacgacggcaccaccaccatcgccaacaacctcacctccaccgtgcaggtgttcaccgactccgagtaccagctcccgtacgtgctcggctccgcccaccagggctgcctcccgccgttcccggccgacgtgttcatggtgccgcagtacggctacctcaccctcaacaacggctcccaggccgtgggcaggtcctccttctactgcctcgagtacttcccgtcccagatgctcaggaccggcaacaacttcaccttctcctacaccttcgaggacgtgccgttccactcctcctacgcccactcccagtccctcgacaggctcatgaacccgctcatcgaccagtacctctactacctctccaggaccaacaccccgtccggcaccaccacccagtccaggctccagttctcccaggccggcgcctccgacatcagggaccagtccaggaactggctcccgggcccgtgctacaggcagcagagggtgtccaagacctccgccgacaacaacaactccgagtactcctggaccggcgccaccaagtaccacctcaacggcagggactccctcgtgaacccgggcccggccatggcctcccacaaggacgacgaggagaagttcttcccgcagtccggcgtgctcatcttcggcaagcagggctccgagaagaccaacgtggacatcgagaaggtgatgatcaccgacgaggaggagatcaggaccaccaacccggtggccaccgagcagtacggctccgtgtccaccaacctccagaggggcaacaggcaggccgccaccgccgacgtgaacacccagggcgtgctcccgggcatggtgtggcaggacagggacgtgtacctccagggcccgatctgggccaagatcccgcacaccgacggccacttccacccgtccccgctcatgggcggcttcggcctcaagcacccgccgccgcagatcctcatcaagaacaccccggtgccggccaacccgtccaccaccttctccgccgccaagttcgcctccttcatcacccagtactccaccggccaggtgtccgtggagatcgagtgggagctccagaaggagaactccaagaggtggaacccggagatccagtacacctccaactacaacaagtccgtgaacgtggacttcaccgtggacaccaacggcgtgtactccgagccgaggccgatcggcaccaggtacctcaccaggaacctctga

SEQ ID NO: 21 - DNA, 2214 bp, Artificial Sequence
  AAV2 CAP optimized for Zea mays
  VP1 Start Codon (7)..(9)
  VP2 Start Codon (418)..(420)
  VP3 Start Codon (613)..(615)
gggtttatgactgccgccggttatctgccggactggctggaggacaccctgtccgagggcatcaggcagtggtggaagctgaagccgggcccgccgccgccgaagccggccgagaggcacaaggacgactccaggggcctggtgctgccgggctacaagtacctgggcccgttcaacggcctggacaagggcgagccggtgaacgaggccgacgccgccgccctggagcacgacaaggcctacgacaggcagctggactccggcgacaacccgtacctgaagtacaaccacgccgacgccgagttccaggagaggctgaaggaggacacctccttcggcggcaacctgggcagggccgtgttccaggccaagaagagggtgctggagccgctgggcctggtggaggagccggtgaagacggccccgggcaagaagaggccggtggagcactccccggtggagccggactcctcctccggcaccggcaaggccggccagcagccggccaggaagaggctgaacttcggccagaccggcgacgccgactccgtgccggacccgcagccgctgggccagccgccggccgccccgtccggcctgggcaccaacaccatggccaccggctccggcgccccgatggccgacaacaacgagggcgccgacggcgtgggcaactcctccggcaactggcactgcgactccacctggatgggcgacagggtgatcaccacctccaccaggacctgggccctgccgacctacaacaaccacctgtacaagcagatctcctcccagtccggcgcctccaacgacaaccactacttcggctactccaccccgtggggctacttcgacttcaacaggttccactgccacttctccccgagggactggcagaggctgatcaacaacaactggggcttcaggccgaagaggctgaacttcaagctgttcaacatccaggtgaaggaggtgacccagaacgacggcaccaccaccatcgccaacaacctgacctccaccgtgcaggtgttcaccgactccgagtaccagctgccgtacgtgctgggctccgcccaccagggctgcctgccgccgttcccggccgacgtgttcatggtgccgcagtacggctacctgaccctgaacaacggctcccaggccgtgggcaggtcctccttctactgcctggagtacttcccgtcccagatgctgaggaccggcaacaacttcaccttctcctacaccttcgaggacgtgccgttccactcctcctacgcccactcccagtccctggacaggctgatgaacccgctgatcgaccagtacctgtactacctgtccaggaccaacaccccgtccggcaccaccacccagtccaggctgcagttctcccaggccggcgcctccgacatcagggaccagtccaggaactggctgccgggcccgtgctacaggcagcagagggtgtccaagacctccgccgacaacaacaactccgagtactcctggaccggcgccaccaagtaccacctgaacggcagggactccctggtgaacccgggcccggccatggcctcccacaaggacgacgaggagaagttcttcccgcagtccggcgtgctgatcttcggcaagcagggctccgagaagaccaacgtggacatcgagaaggtgatgatcaccgacgaggaggagatcaggaccaccaacccggtggccaccgagcagtacggctccgtgtccaccaacctgcagaggggcaacaggcaggccgccaccgccgacgtgaacacccagggcgtgctgccgggcatggtgtggcaggacagggacgtgtacctgcagggcccgatctgggccaagatcccgcacaccgacggccacttccacccgtccccgctgatgggcggcttcggcctgaagcacccgccgccgcagatcctgatcaagaacaccccggtgccggccaacccgtccaccaccttctccgccgccaagttcgcctccttcatcacccagtactccaccggccaggtgtccgtggagatcgagtgggagctgcagaaggagaactccaagaggtggaacccggagatccagtacacctccaactacaacaagtccgtgaacgtggacttcaccgtggacaccaacggcgtgtactccgagccgaggccgatcggcaccaggtacctgaccaggaacctgtga

SEQ ID NO: 22 - DNA, 2214 bp, Artificial Sequence
  AAV2 CAP optimized for Solanum lycopersicoides
  VP1 Start Codon (7)..(9)
  VP2 Start Codon (418)..(420)
  VP3 Start Codon (613)..(615)
gggtttatgactgccgccggttatcttccagattggcttgaggatacactttcagagggaattagacaatggtggaagcttaagccaggaccaccaccaccaaagccagcagagagacataaggatgattcaagaggacttgttcttccaggatacaagtaccttggaccatttaatggacttgataagggagagccagttaatgaggcagatgcagcagcacttgagcatgataaggcatacgatagacaacttgattcaggagataatccataccttaagtacaatcatgcagatgcagagtttcaagagagacttaaggaggatacatcatttggaggaaatcttggaagagcagtttttcaagcaaagaagagagttcttgagccacttggacttgttgaggagccagttaagacggcaccaggaaagaagagaccagttgagcattcaccagttgagccagattcatcatcaggaacaggaaaggcaggacaacaaccagcaagaaagagacttaattttggacaaacaggagatgcagattcagttccagatccacaaccacttggacaaccaccagcagcaccatcaggacttggaacaaatacaatggcaacaggatcaggagcaccaatggcagataataatgagggagcagatggagttggaaattcatcaggaaattggcattgtgattcaacatggatgggagatagagttattacaacatcaacaagaacatgggcacttccaacatacaataatcatctttacaagcaaatttcatcacaatcaggagcatcaaatgataatcattactttggatactcaacaccatggggatactttgattttaatagatttcattgtcatttttcaccaagagattggcaaagacttattaataataattggggatttagaccaaagagacttaattttaagctttttaatattcaagttaaggaggttacacaaaatgatggaacaacaacaattgcaaataatcttacatcaacagttcaagtttttacagattcagagtaccaacttccatacgttcttggatcagcacatcaaggatgtcttccaccatttccagcagatgtttttatggttccacaatacggataccttacacttaataatggatcacaagcagttggaagatcatcattttactgtcttgagtactttccatcacaaatgcttagaacaggaaataattttacattttcatacacatttgaggatgttccatttcattcatcatacgcacattcacaatcacttgatagacttatgaatccacttattgatcaatacctttactacctttcaagaacaaatacaccatcaggaacaacaacacaatcaagacttcaattttcacaagcaggagcatcagatattagagatcaatcaagaaattggcttccaggaccatgttacagacaacaaagagtttcaaagacatcagcagataataataattcagagtactcatggacaggagcaacaaagtaccatcttaatggaagagattcacttgttaatccaggaccagcaatggcatcacataaggatgatgaggagaagttttttccacaatcaggagttcttatttttggaaagcaaggatcagagaagacaaatgttgatattgagaaggttatgattacagatgaggaggagattagaacaacaaatccagttgcaacagagcaatacggatcagtttcaacaaatcttcaaagaggaaatagacaagcagcaacagcagatgttaatacacaaggagttcttccaggaatggtttggcaagatagagatgtttaccttcaaggaccaatttgggcaaagattccacatacagatggacattttcatccatcaccacttatgggaggatttggacttaagcatccaccaccacaaattcttattaagaatacaccagttccagcaaatccatcaacaacattttcagcagcaaagtttgcatcatttattacacaatactcaacaggacaagtttcagttgagattgagtgggagcttcaaaaggagaattcaaagagatggaatccagagattcaatacacatcaaattacaataagtcagttaatgttgattttacagttgatacaaatggagtttactcagagccaagaccaattggaacaagataccttacaagaaatctttga

SEQ ID NO: 23 - DNA, 2214 bp, Artificial Sequence
  AAV2 CAP optimized for Solanum lycopersicum
  VP1 Start Codon (7)..(9)
  VP2 Start Codon (418)..(420)
  VP3 Start Codon (613)..(615)
gggtttatgactgccgccggttatcttcctgattggcttgaagatactctttctgaaggaattagacaatggtggaagcttaagcctggacctcctcctcctaagcctgctgaaagacataaggatgattctagaggacttgttcttcctggatataagtatcttggaccttttaatggacttgataagggagaacctgttaatgaagctgatgctgctgctcttgaacatgataaggcttatgatagacaacttgattctggagataatccttatcttaagtataatcatgctgatgctgaatttcaagaaagacttaaggaagatacttcttttggaggaaatcttggaagagctgtttttcaagctaagaagagagttcttgaacctcttggacttgttgaagaacctgttaagactgctcctggaaagaagagacctgttgaacattctcctgttgaacctgattcttcttctggaactggaaaggctggacaacaacctgctagaaagagacttaattttggacaaactggagatgctgattctgttcctgatcctcaacctcttggacaacctcctgctgctccttctggacttggaactaatactatggctactggatctggagctcctatggctgataataatgaaggagctgatggagttggaaattcttctggaaattggcattgtgattctacttggatgggagatagagttattactacttctactagaacttgggctcttcctacttataataatcatctttataagcaaatttcttctcaatctggagcttctaatgataatcattattttggatattctactccttggggatattttgattttaatagatttcattgtcatttttctcctagagattggcaaagacttattaataataattggggatttagacctaagagacttaattttaagctttttaatattcaagttaaggaagttactcaaaatgatggaactactactattgctaataatcttacttctactgttcaagtttttactgattctgaatatcaacttccttatgttcttggatctgctcatcaaggatgtcttcctccttttcctgctgatgtttttatggttcctcaatatggatatcttactcttaataatggatctcaagctgttggaagatcttctttttattgtcttgaatattttccttctcaaatgcttagaactggaaataattttactttttcttatacttttgaagatgttccttttcattcttcttatgctcattctcaatctcttgatagacttatgaatcctcttattgatcaatatctttattatctttctagaactaatactccttctggaactactactcaatctagacttcaattttctcaagctggagcttctgatattagagatcaatctagaaattggcttcctggaccttgttatagacaacaaagagtttctaagacttctgctgataataataattctgaatattcttggactggagctactaagtatcatcttaatggaagagattctcttgttaatcctggacctgctatggcttctcataaggatgatgaagaaaagttttttcctcaatctggagttcttatttttggaaagcaaggatctgaaaagactaatgttgatattgaaaaggttatgattactgatgaagaagaaattagaactactaatcctgttgctactgaacaatatggatctgtttctactaatcttcaaagaggaaatagacaagctgctactgctgatgttaatactcaaggagttcttcctggaatggtttggcaagatagagatgtttatcttcaaggacctatttgggctaagattcctcatactgatggacattttcatccttctcctcttatgggaggatttggacttaagcatcctcctcctcaaattcttattaagaatactcctgttcctgctaatccttctactactttttctgctgctaagtttgcttcttttattactcaatattctactggacaagtttctgttgaaattgaatgggaacttcaaaaggaaaattctaagagatggaatcctgaaattcaatatacttctaattataataagtctgttaatgttgattttactgttgatactaatggagtttattctgaacctagacctattggaactagatatcttactagaaatctttaa

SEQ ID NO: 24 - DNA, 2214 bp, Artificial Sequence
  AAV2 CAP optimized for Lactuca sativa
  VP1 Start Codon (7)..(9)
  VP2 Start Codon (418)..(420)
  VP3 Start Codon (613)..(615)
gggtttatgactgccgccggttatcttccagattggcttgaagatacactttctgaaggaattagacaatggtggaaacttaaaccaggaccaccaccaccaaaaccagctgaaagacataaagatgattctagaggacttgttcttccaggatataaatatcttggaccatttaatggacttgataaaggagaaccagttaatgaagctgatgctgctgctcttgaacatgataaagcttatgatagacaacttgattctggagataatccatatcttaaatataatcatgctgatgctgaatttcaagaaagacttaaagaagatacatcttttggaggaaatcttggaagagctgtttttcaagctaaaaaaagagttcttgaaccacttggacttgttgaagaaccagttaaaacagctccaggaaaaaaaagaccagttgaacattctccagttgaaccagattcttcttctggaacaggaaaagctggacaacaaccagctagaaaaagacttaattttggacaaacaggagatgctgattctgttccagatccacaaccacttggacaaccaccagctgctccatctggacttggaacaaatacaatggctacaggatctggagctccaatggctgataataatgaaggagctgatggagttggaaattcttctggaaattggcattgtgattctacatggatgggagatagagttattacaacatctacaagaacatgggctcttccaacatataataatcatctttataaacaaatttcttctcaatctggagcttctaatgataatcattattttggatattctacaccatggggatattttgattttaatagatttcattgtcatttttctccaagagattggcaaagacttattaataataattggggatttagaccaaaaagacttaattttaaactttttaatattcaagttaaagaagttacacaaaatgatggaacaacaacaattgctaataatcttacatctacagttcaagtttttacagattctgaatatcaacttccatatgttcttggatctgctcatcaaggatgtcttccaccatttccagctgatgtttttatggttccacaatatggatatcttacacttaataatggatctcaagctgttggaagatcttctttttattgtcttgaatattttccatctcaaatgcttagaacaggaaataattttacattttcttatacatttgaagatgttccatttcattcttcttatgctcattctcaatctcttgatagacttatgaatccacttattgatcaatatctttattatctttctagaacaaatacaccatctggaacaacaacacaatctagacttcaattttctcaagctggagcttctgatattagagatcaatctagaaattggcttccaggaccatgttatagacaacaaagagtttctaaaacatctgctgataataataattctgaatattcttggacaggagctacaaaatatcatcttaatggaagagattctcttgttaatccaggaccagctatggcttctcataaagatgatgaagaaaaattttttccacaatctggagttcttatttttggaaaacaaggatctgaaaaaacaaatgttgatattgaaaaagttatgattacagatgaagaagaaattagaacaacaaatccagttgctacagaacaatatggatctgtttctacaaatcttcaaagaggaaatagacaagctgctacagctgatgttaatacacaaggagttcttccaggaatggtttggcaagatagagatgtttatcttcaaggaccaatttgggctaaaattccacatacagatggacattttcatccatctccacttatgggaggatttggacttaaacatccaccaccacaaattcttattaaaaatacaccagttccagctaatccatctacaacattttctgctgctaaatttgcttcttttattacacaatattctacaggacaagtttctgttgaaattgaatgggaacttcaaaaagaaaattctaaaagatggaatccagaaattcaatatacatctaattataataaatctgttaatgttgattttacagttgatacaaatggagtttattctgaaccaagaccaattggaacaagatatcttacaagaaatctttga

SEQ ID NO: 25 - protein, 735 aa, adeno-associated virus 2 [sequence omitted]

SEQ ID NO: 26 - protein, 735 aa, Artificial Sequence
  AAV2 CAP optimized for plant expression
MTAAGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 27 - DNA, 618 bp, adeno-associated virus 2
atgctggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctga

SEQ ID NO: 28 - DNA, 618 bp, Artificial Sequence
  AAV2 AAP optimized for Nicotiana benthamiana
atgttagagactcagacacaatacttgactccatcactttcagatagccatcagcagcctccactcgtttgggaactcataaggtggcttcaagctgttgctcatcaatggcaaacaattactagggctcctacagaatgggttattccaagagagattggaattgctattcctcatggttgggctactgaatcttcaccacctgctccagagcctggaccatgtccacctactacaactacatcaacaaataagtttcctgctaatcaagaaccaagaactacaattactacacttgctactgctcctcttggaggtattttgacatcaactgattctacagctacttttcatcatgttacaggaaaagattcttcaactacaactggagattcagatccaagggattctacatcttcatctttgacttttaagtcaaaaagatctagaaggatgacagttagaaggagacttcctattactttgccagctaggtttagatgtcttttgacaaggtctacttcatctagaacttcatctgctaggagaattaaggatgcaagcagaagaagccaacagacaagttcctggtgccacagcatggatacaagcccttaa

SEQ ID NO: 29 - DNA, 618 bp, Artificial Sequence
  AAV2 AAP optimized for Arabidopsis thaliana
atgcttgaaactcaaactcaatatcttactccttctctttctgattctcatcaacaacctcctcttgtttgggaacttattagatggcttcaagctgttgctcatcaatggcaaactattactagagctcctactgaatgggttattcctagagaaattggaattgctattcctcatggatgggctactgaatcttctcctcctgctcctgaacctggaccttgtcctcctactactactacttctactaataagtttcctgctaatcaagaacctagaactactattactactcttgctactgctcctcttggaggaattcttacttctactgattctactgctacttttcatcatgttactggaaaggattcttctactactactggagattctgatcctagagattctacttcttcttctcttacttttaagtctaagagatctagaagaatgactgttagaagaagacttcctattactcttcctgctagatttagatgtcttcttactagatctacttcttctagaacttcttctgctagaagaattaaggatgcttctagaagatctcaacaaacttcttcttggtgtcattctatggatacttctccttga

SEQ ID NO: 30 - DNA, 618 bp, Artificial Sequence
  AAV2 AAP optimized for Solanum tuberosum
atgcttgaaactcaaactcaatatcttactccttctctttctgattctcatcaacaacctcctcttgtttgggaacttattagatggcttcaagctgttgctcatcaatggcaaactattactagagctcctactgaatgggttattcctagagaaattggaattgctattcctcatggatgggctactgaatcttctcctcctgctcctgaacctggaccttgtcctcctactactactacttctactaataagtttcctgctaatcaagaacctagaactactattactactcttgctactgctcctcttggaggaattcttacttctactgattctactgctacttttcatcatgttactggaaaggattcttctactactactggagattctgatcctagagattctacttcttcttctcttacttttaagtctaagagatctagaagaatgactgttagaagaagacttcctattactcttcctgctagatttagatgtcttcttactagatctacttcttctagaacttcttctgctagaagaattaaggatgcttctagaagatctcaacaaacttcttcttggtgtcattctatggatacttctccttaa

SEQ ID NO: 31 - DNA, 618 bp, Artificial Sequence
  AAV2 AAP optimized for Cannabis sativa
atgttggaaactcaaactcaatatttgactccttcattgtcagattcacatcaacaacctcctttggtttgggaattgattagatggttgcaagctgttgctcatcaatggcaaactattactagagctcctactgaatgggttattcctagagaaattggaattgctattcctcatggatgggctactgaatcatcacctcctgctcctgaacctggaccttgccctcctactactactacttcaactaataaatttcctgctaatcaagaacctagaactactattactactttggctactgctcctttgggaggaattttgacttcaactgattcaactgctacttttcatcatgttactggaaaagattcatcaactactactggagattcagatcctagagattcaacttcatcatcattgacttttaaatcaaaaagatcaagaagaatgactgttagaagaagattgcctattactttgcctgctagatttagatgcttgttgactagatcaacttcatcaagaacttcatcagctagaagaattaaagatgcttcaagaagatcacaacaaacttcatcatggtgccattcaatggatacttcaccttaa

SEQ ID NO: 32 - DNA, 618 bp, Artificial Sequence
  AAV2 AAP optimized for Fagopyrum esculentum
atgctcgagacccagacccagtacctcaccccttccctctccgattcccatcagcagcctcctctcgtttgggagctcatcaggtggctccaggctgttgctcatcagtggcagaccatcaccagggctcctaccgagtgggttatccctagggagatcggaatcgctatccctcatggatgggctaccgagtcctcccctcctgctcctgagcctggaccttgccctcctaccaccaccacctccaccaacaagttccctgctaaccaggagcctaggaccaccatcaccaccctcgctaccgctcctctcggaggaatcctcacctccaccgattccaccgctaccttccatcatgttaccggaaaggattcctccaccaccaccggagattccgatcctagggattccacctcctcctccctcaccttcaagtccaagaggtccaggaggatgaccgttaggaggaggctccctatcaccctccctgctaggttcaggtgcctcctcaccaggtccacctcctccaggacctcctccgctaggaggatcaaggatgcttccaggaggtcccagcagacctcctcctggtgccattccatggatacctccccttaa

SEQ ID NO: 33 - DNA, 618 bp, Artificial Sequence
  AAV2 AAP optimized for Oryza sativa
atgctcgagacccagacccagtacctcaccccgtccctctccgactcccaccagcagccgccgctcgtgtgggagctcatcaggtggctccaggccgtggcccaccagtggcagaccatcaccagggccccgaccgagtgggtgatcccgagggagatcggcatcgccatcccgcacggctgggccaccgagtcctccccgccggccccggagccgggcccgtgcccgccgaccaccaccacctccaccaacaagttcccggccaaccaggagccgaggaccaccatcaccaccctcgccaccgccccgctcggcggcatcctcacctccaccgactccaccgccaccttccaccacgtgaccggcaaggactcctccaccaccaccggcgactccgacccgagggactccacctcctcctccctcaccttcaagtccaagaggtccaggaggatgaccgtgaggaggaggctcccgatcaccctcccggccaggttcaggtgcctcctcaccaggtccacctcctccaggacctcctccgccaggaggatcaaggacgcctccaggaggtcccagcagacctcctcctggtgccactccatggacacctccccgtga

SEQ ID NO: 34 - DNA, 618 bp, Artificial Sequence
  AAV2 AAP optimized for Zea mays
atgctggagacccagacccagtacctgaccccgagcctgagcgacagccaccagcagccgccgctggtgtgggagctgatcaggtggctgcaggccgtggcccaccagtggcagaccatcaccagggccccgaccgagtgggtgatcccgagggagatcggcatcgccatcccgcacggctgggccaccgagagcagcccgccggccccggagccgggcccgtgcccgccgaccaccaccaccagcaccaacaagttcccggccaaccaggagccgaggaccaccatcaccaccctggccaccgccccgctgggcggcatcctgaccagcaccgacagcaccgccaccttccaccacgtgaccggcaaggacagcagcaccaccaccggcgacagcgacccgagggacagcaccagcagcagcctgaccttcaagagcaagaggagcaggaggatgaccgtgaggaggaggctgccgatcaccctgccggccaggttcaggtgcctgctgaccaggagcaccagcagcaggaccagcagcgccaggaggatcaaggacgccagcaggaggagccagcagaccagctcctggtgccacagcatggacaccagcccgtga

SEQ ID NO: 35 - DNA, 618 bp, Artificial Sequence
  AAV2 AAP optimized for Solanum lycopersicoides
atgcttgagacacaaacacaataccttacaccatcactttcagattcacatcaacaaccaccacttgtttgggagcttattagatggcttcaagcagttgcacatcaatggcaaacaattacaagagcaccaacagagtgggttattccaagagagattggaattgcaattccacatggatgggcaacagagtcatcaccaccagcaccagagccaggaccatgtccaccaacaacaacaacatcaacaaataagtttccagcaaatcaagagccaagaacaacaattacaacacttgcaacagcaccacttggaggaattcttacatcaacagattcaacagcaacatttcatcatgttacaggaaaggattcatcaacaacaacaggagattcagatccaagagattcaacatcatcatcacttacatttaagtcaaagagatcaagaagaatgacagttagaagaagacttccaattacacttccagcaagatttagatgtcttcttacaagatcaacatcatcaagaacatcatcagcaagaagaattaaggatgcatcaagaagatcacaacaaacatcatcatggtgtcattcaatggatacatcaccatga

SEQ ID NO: 36 - DNA, 618 bp, Artificial Sequence; identical to SEQ ID NO: 30
  AAV2 AAP optimized for Solanum lycopersicum

SEQ ID NO: 37 - DNA, 618 bp, Artificial Sequence
  AAV2 AAP optimized for Lactuca sativa
atgcttgaaacacaaacacaatatcttacaccatctctttctgattctcatcaacaaccaccacttgtttgggaacttattagatggcttcaagctgttgctcatcaatggcaaacaattacaagagctccaacagaatgggttattccaagagaaattggaattgctattccacatggatgggctacagaatcttctccaccagctccagaaccaggaccatgtccaccaacaacaacaacatctacaaataaatttccagctaatcaagaaccaagaacaacaattacaacacttgctacagctccacttggaggaattcttacatctacagattctacagctacatttcatcatgttacaggaaaagattcttctacaacaacaggagattctgatccaagagattctacatcttcttctcttacatttaaatctaaaagatctagaagaatgacagttagaagaagacttccaattacacttccagctagatttagatgtcttcttacaagatctacatcttctagaacatcttctgctagaagaattaaagatgcttctagaagatctcaacaaacatcttcttggtgtcattctatggatacatctccatga

SEQ ID NO: 38 - protein, 205 aa, adeno-associated virus 2
MLETQTQYLTPSLSDSHQQPPLVWELIRWLQAVAHQWQTITRAPTEWVIPREIGIAIPHGWATESSPPAPEPGPCPPTTTTSTNKFPANQEPRTTITTLATAPLGGILTSTDSTATFHHVTGKDSSTTTGDSDPRDSTSSSLTFKSKRSRRMTVRRRLPITLPARFRCLLTRSTSSRTSSARRIKDASRRSQQTSSWCHSMDTSP

SEQ ID NO: 39 - DNA, 453 bp, adeno-associated virus 2
atgaccaccagcggcgtgcccttcggcatgaccctgagacccaccagaagcagactgagcagaagaaccccctacagcagagacagactgccccccttcgagaccgagaccagagccaccatcctggaggaccaccccctgctgcccgagtgcaacaccctgaccatgcacaacgcctggaccagccccagcccccccgtgaagcagccccaggtgggccagcagcccgtggcccagcagctggacagcgacatgaacctgagcgagctgcccggcgagttcatcaacatcaccgacgagagactggccagacaggagaccgtgtggaacatcacccccaagaacatgagcgtgacccacgacatgatgctgttcaaggccagcagaggcgagagaaccgtgtacagcgtgtgctgggagggcggcggcagactgaacaccagagtgctgtaa

SEQ ID NO: 40 - DNA, 453 bp, Artificial Sequence
  Ad5 E4orf6 optimized for Nicotiana benthamiana
atgactacatctggtgttccatttggaatgactcttagacctacaagatctaggttgtcaagaaggacaccatattcaagagataggcttccaccttttgaaactgagacaagggctactattttggaagatcatccacttttgcctgagtgtaatactcttacaatgcataatgcttggacatctccttcaccacctgttaagcaaccacaagttggtcaacaacctgttgctcaacaattggattctgatatgaatctttcagaattgccaggagagtttattaatatcactgatgaaagacttgctaggcaagagactgtttggaacatcacacctaagaacatgtctgttactcatgatatgatgttgtttaaagcttctagaggtgaaaggacagtttactcagtttgttgggagggaggtggaagacttaatactagggttttgtaa

SEQ ID NO: 41 - DNA, 453 bp, Artificial Sequence
  Ad5 E4orf6 optimized for Arabidopsis thaliana
atgactacttctggagttccttttggaatgactcttagacctactagatctagactttctagaagaactccttattctagagatagacttcctccttttgaaactgaaactagagctactattcttgaagatcatcctcttcttcctgaatgtaatactcttactatgcataatgcttggacttctccttctcctcctgttaagcaacctcaagttggacaacaacctgttgctcaacaacttgattctgatatgaatctttctgaacttcctggagaatttattaatattactgatgaaagacttgctagacaagaaactgtttggaatattactcctaagaatatgtctgttactcatgatatgatgctttttaaggcttctagaggagaaagaactgtttattctgtttgttgggaaggaggaggaagacttaatactagagttctttga

SEQ ID NO: 42 - DNA, 453 bp, Artificial Sequence
  Ad5 E4orf6 optimized for Solanum tuberosum
atgactacttctggagttccttttggaatgactcttagacctactagatctagactttctagaagaactccttattctagagatagacttcctccttttgaaactgaaactagagctactattcttgaagatcatcctcttcttcctgaatgtaatactcttactatgcataatgcttggacttctccttctcctcctgttaagcaacctcaagttggacaacaacctgttgctcaacaacttgattctgatatgaatctttctgaacttcctggagaatttattaatattactgatgaaagacttgctagacaagaaactgtttggaatattactcctaagaatatgtctgttactcatgatatgatgctttttaaggcttctagaggagaaagaactgtttattctgtttgttgggaaggaggaggaagacttaatactagagttctttaa

SEQ ID NO: 43 - DNA, 453 bp, Artificial Sequence
  Ad5 E4orf6 optimized for Cannabis sativa
atgactacttcaggagttccttttggaatgactttgagacctactagatcaagattgtcaagaagaactccttattcaagagatagattgcctccttttgaaactgaaactagagctactattttggaagatcatcctttgttgcctgaatgcaatactttgactatgcataatgcttggacttcaccttcacctcctgttaaacaacctcaagttggacaacaacctgttgctcaacaattggattcagatatgaatttgtcagaattgcctggagaatttattaatattactgatgaaagattggctagacaagaaactgtttggaatattactcctaaaaatatgtcagttactcatgatatgatgttgtttaaagcttcaagaggagaaagaactgtttattcagtttgctgggaaggaggaggaagattgaatactagagttttgtaa

SEQ ID NO: 44 - DNA, 453 bp, Artificial Sequence
  Ad5 E4orf6 optimized for Fagopyrum esculentum
atgaccacctccggagttcctttcggaatgaccctcaggcctaccaggtccaggctctccaggaggaccccttactccagggacaggctccctcctttcgagaccgagaccagggccaccatcctcgaggaccatcctctcctccctgagtgcaacaccctcaccatgcataacgcctggacctccccttcccctcctgttaagcagcctcaggttggacagcagcctgttgcccagcagctcgactccgacatgaacctctccgagctccctggagagttcatcaacatcaccgacgagaggctcgccaggcaggagaccgtttggaacatcacccctaagaacatgtccgttacccatgacatgatgctcttcaaggcctccaggggagagaggaccgtttactccgtttgctgggagggaggaggaaggctcaacaccagggttctctaa

SEQ ID NO: 45 - DNA, 453 bp, Artificial Sequence
  Ad5 E4orf6 optimized for Oryza sativa
atgaccacctccggcgtgccgttcggcatgaccctcaggccgaccaggtccaggctctccaggaggaccccgtactccagggacaggctcccgccgttcgagaccgagaccagggccaccatcctcgaggaccacccgctcctcccggagtgcaacaccctcaccatgcacaacgcctggacctccccgtccccgccggtgaagcagccgcaggtgggccagcagccggtggcccagcagctcgactccgacatgaacctctccgagctcccgggcgagttcatcaacatcaccgacgagaggctcgccaggcaggagaccgtgtggaacatcaccccgaagaacatgtccgtgacccacgacatgatgctcttcaaggcctccaggggcgagaggaccgtgtactccgtgtgctgggagggcggcggcaggctcaacaccagggtgctctga

SEQ ID NO: 46 - DNA, 453 bp, Artificial Sequence
  Ad5 E4orf6 optimized for Zea mays
atgaccaccagcggcgtgccgttcggcatgaccctgaggccgaccaggagcaggctgagcaggaggaccccgtacagcagggacaggctgccgccgttcgagaccgagaccagggccaccatcctggaggaccacccgctgctgccggagtgcaacaccctgaccatgcacaacgcctggaccagcccgagcccgccggtgaagcagccgcaggtgggccagcagccggtggcccagcagctggacagcgacatgaacctgagcgagctgccgggcgagttcatcaacatcaccgacgagaggctggccaggcaggagaccgtgtggaacatcaccccgaagaacatgagcgtgacccacgacatgatgctgttcaaggccagcaggggcgagaggaccgtgtacagcgtgtgctgggagggcggcggcaggctgaacaccagggtgctgtga

SEQ ID NO: 47 - DNA, 453 bp, Artificial Sequence
  Ad5 E4orf6 optimized for Solanum lycopersicoides
atgacaacatcaggagttccatttggaatgacacttagaccaacaagatcaagactttcaagaagaacaccatactcaagagatagacttccaccatttgagacagagacaagagcaacaattcttgaggatcatccacttcttccagagtgtaatacacttacaatgcataatgcatggacatcaccatcaccaccagttaagcaaccacaagttggacaacaaccagttgcacaacaacttgattcagatatgaatctttcagagcttccaggagagtttattaatattacagatgagagacttgcaagacaagagacagtttggaatattacaccaaagaatatgtcagttacacatgatatgatgctttttaaggcatcaagaggagagagaacagtttactcagtttgttgggagggaggaggaagacttaatacaagagttctttga

SEQ ID NO: 48 - DNA, 453 bp, Artificial Sequence; identical to SEQ ID NO: 42
  Ad5 E4orf6 optimized for Solanum lycopersicum

SEQ ID NO: 49 - DNA, 453 bp, Artificial Sequence
  Ad5 E4orf6 optimized for Lactuca sativa
atgacaacatctggagttccatttggaatgacacttagaccaacaagatctagactttctagaagaacaccatattctagagatagacttccaccatttgaaacagaaacaagagctacaattcttgaagatcatccacttcttccagaatgtaatacacttacaatgcataatgcttggacatctccatctccaccagttaaacaaccacaagttggacaacaaccagttgctcaacaacttgattctgatatgaatctttctgaacttccaggagaatttattaatattacagatgaaagacttgctagacaagaaacagtttggaatattacaccaaaaaatatgtctgttacacatgatatgatgctttttaaagcttctagaggagaaagaacagtttattctgtttgttgggaaggaggaggaagacttaatacaagagttctttga

SEQ ID NO: 50 - protein, 150 aa, adeno-associated virus 2
MTTSGVPFGMTLRPTRSRLSRRTPYSRDRLPPFETETRATILEDHPLLPECNTLTMHNAWTSPSPPVKQPQVGQQPVAQQLDSDMNLSELPGEFINITDERLARQETVWNITPKNMSVTHDMMLFKASRGERTVYSVCWEGGGRLNTRVL

SEQ ID NO: 51 - DNA, 2729 bp, Artificial Sequence
  AAV reporter construct optimized for Nicotiana benthamiana
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagaggatccggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctttcacgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcgccaccatggtttctaaaggagaagagctttttacaggtgttgttccaattcttgttgagttggatggagatgttaatggtcataagttttctgtttcaggagaaggagagggagatgctacttacggaaagcttacattgaagtttatttgtactacaggaaagcttccagttccttggccaactcttgttactacattgacatatggagttcaatgtttttcaaggtaccctgatcatatgaagcaacatgatttctttaagtctgctatgccagaaggatatgttcaagagagaactattttctttaaggatgatggtaactacaaaactagggctgaggttaagtttgagggagatacattggttaacagaatcgaacttaagggtatcgatttcaaggaggatggaaacatccttggtcataagttggaatacaactacaactcacataacgtttacatcatggctgataagcaaaagaatggtattaaggttaacttcaagatcagacataatattgaggatggttctgttcaacttgctgatcattaccaacaaaacactcctattggagatggacctgttcttttgccagataatcattacttgtctacacaatcagctctttctaaggatccaaatgagaaaagggatcatatggttcttttggagtttgttactgctgctggaatcacacttggtatggatgaattgtataagtcaggtcttagatcttactaataggattttaaacggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 52 - DNA, 21 bp, Artificial Sequence
  ITR forward PCR primer
ggaacccctagtgatggagtt

SEQ ID NO: 53 - DNA, 16 bp, Artificial Sequence
  ITR reverse PCR primer
cggcctcagtgagcga

SEQ ID NO: 54 - DNA, 15 bp, Artificial Sequence
  Plant engineered Kozak for AAV2 REP
gggtttatgactggt

SEQ ID NO: 55 - DNA, 25 bp, Artificial Sequence
  Plant engineered Kozak for AAV2 CAP
gggtttatgactggccgccggttat

(19) United States
(12) Patent Application Publication
(10) Pub. No.: US 2023/0087751 A1
(43) Pub. Date: Mar. 23, 2023

(54) RECOMBINANT ADENO-ASSOCIATED VIRAL VECTORS IN PLANTS

(71) Applicant: VECPROBIO, INC., Del Mar, CA (US)

(72) Inventors: Daniel Gibbs, Del Mar, CA (US); Jake Orion Connors, Del Mar, CA (US)

(21) Appl. No.: 17/795,822

(22) PCT Filed: Feb. 3, 2021
(86) PCT No.: PCT/US2021/016393

§ 371 Date: Jul. 27, 2022

Related U.S. Application Data
(60) Provisional application No. 62/971,750, filed on Feb. 7, 2020.

(51) Int. Cl. C12N 15/86; C12N 15/82; C07K 14/005

(52) U.S. Cl. CPC C12N 15/86; C12N 15/8205; C07K 14/005; C12N 2750/14143; C12N 2750/14122; C12N 2750/14152

(57) ABSTRACT

The present disclosure relates to nucleic acid sequences encoding components of adeno-associated virus (AAV), such as those that have been codon optimized for expression in plants, and the proteins that are expressed from these nucleic acid sequence. Also disclosed are methods of producing functional AAV particles using these nucleic acid sequences in plants. Production of AAV in plants as disclosed herein offer many advantages over conventional processes, such as efficiency, cost, yield, scalability, and safety.

### CROSS-REFERENCE TO RELATED APPLICATIONS

This application claims the benefit of priority of U.S. Provisional Patent Application No. 62/971,750, filed Feb. 7, 2020, which is hereby expressly incorporated by reference in its entirety.

### REFERENCE TO SEQUENCE LISTING

The present application is being filed along with a Sequence Listing in electronic format. The Sequence Listing is provided in a file entitled SeqListingVCPRO002WO.TXT, which was created on Feb. 3, 2021 and is 115,770 bytes in size. The information in the electronic Sequence Listing is hereby incorporated by reference in its entirety.

### FIELD OF THE INVENTION

The present disclosure relates to nucleic acid sequences encoding components of adeno-associated virus (AAV), such as those that have been codon optimized for expression in plants, and the proteins that are expressed from these nucleic acid sequence. Also disclosed are methods of producing functional AAV particles using these nucleic acid sequences in plants. Production of AAV in plants as disclosed herein offer many benefits as compared to conventional processes of virus production, including efficiency, cost, purity, yield, scalability, and safety.

### BACKGROUND OF THE INVENTION

Adeno-associated viruses (AAV) have found great popularity for use in both in vitro transduction into human cells and in vivo transduction for gene therapy due to its minimal immunogenicity, high efficacy, and relative safety. AAV particles are typically produced in mammalian or insect cell culture systems but maintaining these cell cultures, purification of the AAV particles, and obtaining sufficient viral titers is difficult and expensive. There is a present need for improved methods for producing AAV particles.

### SUMMARY OF THE INVENTION

Described herein are embodiments directed to nucleic acids comprising, consisting essentially of, or consisting of sequences that encode adeno-associated virus (AAV) proteins. In some embodiments, the AAV is an AAV serotype 1, 2, 3, 4, 5, 6, 7, 8, 9, 10, 11 or 12. In some embodiments, the AAV is AAV serotype 2 (AAV2), which is a serotype commonly used in research and clinical applications. AAV proteins include but are not limited to REP proteins, REP78, REP68, REP52, REP40, CAP proteins, VP1, VP2, VP3, or AAP. Adenovirus proteins, which may enhance the replication of AAV in host cells, include but are not limited to E4orf6, Ela, E2a, E2b and VA. In some embodiments, the nucleic acids comprising, consisting essentially of, or consisting of sequences that encode for AAV proteins are transcribed and translated into AAV proteins in a live host or a cell-free system. In other embodiments, the nucleic acids comprising, consisting essentially of, or consisting of sequences that encode for AAV proteins have at least 90%, 91%, 92%, 93%, 94%, 95%, 96%, 97%, 98%, 99%, or 100% sequence identity to wild type sequences that encode for the AAV proteins. In some embodiments, the nucleic acids have at least at least 90%, 91%, 92%, 93%, 94%, 95%, 96%, 97%, 98%, 99%, or 100% sequence identity to wild type sequences that encode for wild type AAV2 proteins. In some embodiments, the nucleic acids are codon optimized for improved, increased, or enhanced expression in a plant. In some embodiments, the nucleic acids have at least 90%, 91%, 92%, 93%, 94%, 95%, 96%, 97%, 98%, 99%, or 100% sequence identity to SEQ ID NOs: 2-11 and encode an AAV2 REP/REP78/REP/68/REP52/REP48 protein, to SEQ ID NOs: 15-24 and encode an AAV2 CAP/VP1/VP2/VP3 protein, to SEQ ID NOs: 28-37 and encode an AAV2 AAP protein, or to SEQ ID NOs: 40-49 and encode an Ad5 E4orf6 protein. In some embodiments, the nucleic acids are codon optimized for expression in Nicotiana benthamiana, Nicotiana tabacum, Arabidopsis thaliana, Solanum tuberosum, Cannabis sativa, Fagopyrum esculentum, Oryza sativa, Zea mays, Solanum lycopersicoides, Solanum lycopersicum, Lactuca sativa. In some embodiments, a recombinant nucleic acid vector, including but not limited to a pEAQ vector, an AAV particle, an Agrobacterium tumefaciens cell, a plant cell, or a plant comprises the nucleic acids that encode for AAV proteins. Additionally described are methods for isolating AAV particles from a plant, wherein the plant may belong to the genera Nicotiana, Arabidopsis, Solanum, Cannabis, Fagopyrum, Oryza, Lactuca or Zea. In some embodiments, the AAV particles are isolated from a plant by a method comprising centrifugation, filtration, chromatography, affinity chromatography, ion exchange chromatography, anion exchange chromatography, size exclusion chromatography, or hydrophobic interaction chromatography.

In some embodiments, the purified AAV particles are used as a medicament. In some embodiments, the purified AAV particles are used in the manufacture of a medicament. In some embodiments, the purified AAV particles are used to infect a mammalian host cell, such as a human host cell. In some embodiments, the purified AAV particles are used to treat a disease. In some embodiments, the purified AAV particles are used for gene therapy for a patient in need of a therapeutic protein or peptide, such as a human patient. In some embodiments, the purified AAV particles are used to treat inborn errors in metabolism including but not limited to enzyme deficiencies, glycogen storage disease (GSD), GSD type 0, GSD type I, GSD type II, Pompe disease, Danon disease, GSD type III, GSD type IV, GSD type V, GSD type VI, GSD type VII, GSD type VIII, GSD type IX, congenital alactasia, sucrose intolerance, fructosuria, fructose intolerance, galactokinase deficiency, galactosemia, adult polyglucosan body disease, diabetes, hyperinsulinemic hypoglycemia, triosephosphate isomerase deficiency, pyruvate kinase deficiency, pyruvate carboxylate deficiency, fructose bisphosphate deficiency, glucose-6-phosphate dehydrogenase deficiency, transaldolase deficiency, 6-phosphonogluconate dehydrogenase deficiency, hyperoxaluria, pentosuria, or aldolase A deficiency. In some embodiments, the purified AAV particles are used to treat neurological or neurodegenerative disorders, including but not limited to amyotrophic lateral sclerosis, spinal muscular atrophy, Parkinson's disease, Alzheimer's disease, motor neuron disease, muscular dystrophies, Becker muscular dystrophy, Duchenne muscular dystrophy, mucopolysaccharidosis IIIB, or aromatic L-amino acid decarboxylase deficiency. In some embodiments, the purified AAV particles are used to treat retinal degenerative diseases including but not limited to retinitis pigmentosa, Usher syndrome, Stargardt disease, choroideremia, achromatopsia, or X-linked retinoschisis. In some embodiments, the purified AAV particles are used to treat blood disorders, including but not limited to β-thalassemia, sickle cell disease, or hemophilia. In some embodiments, the purified AAV particles are used to treat hereditary or congenital causes of deafness. In some embodiments, the purified AAV particles are used to treat Wiskott-Aldrich syndrome, X-linked chronic granulomatous disease, recessive dystrophic epidermolysis bullosa, mucopolysaccharidosis type I, alpha 1 antitrypsin deficiency, or homozygous familial hypercholesterolemia.

In some embodiments, plants are prepared with hydroponics. In some embodiments, plant seeds are prepared in Grodan rockwool cubes soaked in fertilizer solution with humidity to germinate. In some embodiments, germinating seeds or plants are held under a light cycle, such as 16 hours light/8 hours dark, 24 hours light/0 hours dark, 12 hours light/12 hours dark, or 18 hours light/6 hours dark. In some embodiments, germinating seeds or plants are held at an appropriate temperature, such as 50, 55, 60, 65, 70, 75, 80, 85, 90, 95, or 100 degrees Fahrenheit or any temperature within a range defined by any two of the aforementioned temperatures. In some embodiments, the seeds germinate within 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. In some embodiments, the growing plant should be transferred to a bigger container within 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days once roots being protruding.

In some embodiments, nucleic acid plasmids, constructs, or vectors comprising AAV2 genes are assembled. In some embodiments, these nucleic acid plasmids, constructs, or vectors comprising AAV2 genes include pEAQ-HT-Ad5Orf6-OPT_AAV2-AAP-OPT, pEAQ-HT_CAPopt, or pEAQ-HT-REPopt_AVGFPopt. In some embodiments, these plasmids, constructs, or vectors are transformed into A. tumefaciens. In some embodiments, transformed A. tumefaciens are grown in cultures appropriate for scale, such as 10 mL, 20 mL, 30 mL, 40 mL, 50 mL, 100 mL, 200 mL, 300 mL, 400 mL, 500 mL, 1 L, 2 L, 3 L, 4 L, 5 L, 10 L, 20 L, 30 L, 40 L, 50 L, 100 L, 1000 L, 5000 L, 10000 L, 50000 L, 100000 L, 1000000 L or any volume within a range defined by any two of the aforementioned volumes. In some embodiments, plants are agroinfiltrated with cultures of transformed A. tumefaciens. In some embodiments, agroinfiltrated plants produce AAV2 particles within the cells of the plant. In some embodiments, parts of the plant, such as the leaves, stems, flowers, roots, or fruits are removed for processing to purify the AAV2 particles.

In some embodiments, AAV2 particles are processed from biological material using centrifugation, chromatography, filtration, or other method. In some embodiments, at least 10⁴, 10⁵, 10⁶, 10⁷, 10⁸, 10⁹, 10¹⁰, 10¹¹, 10¹², 10¹³, or 10¹⁴ viral particles or viral genomes are purified from each plant. In some embodiments, intact viral particles make up at least 40%, 50%, 60%, 70%, 80%, 90%, 91%, 92%, 93%, 94%, 95%, 96%, 97%, 98%, 99%, or 100% of the total viral particles purified. In some embodiments, the viral particles are 50%, 60%, 70%, 80%, 90%, 91%, 92%, 93%, 94%, 95%, 96%, 97%, 98%, 99%, or 100% pure. In some embodiments, these purified viral particles are used for transduction, research, gene therapy, or a therapeutic purpose.

Preferred aspects of the present invention relate to the following numbered alternatives:

1. A nucleic acid molecule comprising a sequence that encodes an AAV2 REP protein, wherein the sequence has at least 90%, 91%, 92%, 93%, 94%, 95%, 96%, 97%, 98%, 99%, or 100% sequence identity to SEQ ID NO: 2-11.

2. The nucleic acid molecule of claim 1, wherein the sequence has at least 90%, 91%, 92%, 93%, 94%, 95%, 96%, 97%, 98%, 99%, or 100% sequence identity to SEQ ID NO: 2.

3. A nucleic acid molecule comprising a sequence that encodes an AAV2 CAP protein, wherein the sequence has at least 90%, 91%, 92%, 93%, 94%, 95%, 96%, 97%, 98%, 99%, or 100% sequence identity to SEQ ID NO: 15-24.

4. The nucleic acid molecule of claim 3, wherein the sequence has at least 90%, 91%, 92%, 93%, 94%, 95%, 96%, 97%, 98%, 99%, or 100% sequence identity to SEQ ID NO: 15.

5. A nucleic acid molecule comprising a sequence that encodes an AAV2 AAP protein, wherein the sequence has at least 90%, 91%, 92%, 93%, 94%, 95%, 96%, 97%, 98%, 99%, or 100% sequence identity to SEQ ID NO: 28-37.

6. The nucleic acid molecule of claim 5, wherein the sequence has at least 90%, 91%, 92%, 93%, 94%, 95%, 96%, 97%, 98%, 99%, or 100% sequence identity to SEQ ID NO: 28.

7. A nucleic acid molecule comprising a sequence that encodes an Ad5 E4orf6 protein, wherein the sequence has at least 90%, 91%, 92%, 93%, 94%, 95%, 96%, 97%, 98%, 99%, or 100% sequence identity to SEQ ID NO: 40-49.

8. The nucleic acid molecule of claim 7, wherein the sequence has at least 90%, 91%, 92%, 93%, 94%, 95%, 96%, 97%, 98%, 99%, or 100% sequence identity to SEQ ID NO: 40.

9. A recombinant nucleic acid vector comprising a nucleic acid molecule of any one of claims 1-8.

10. A protein encoded by any one of the nucleic acids of any one of claims 1-8 or the vector of claim 10.

11. An AAV particle comprising at least one nucleic acid molecule of any one of claims 1-8, the vector of claim 9, or the protein of claim 10.

12. A plant cell comprising at least one nucleic acid molecule of any one of claims 1-8, the recombinant nucleic acid vector of claim 9, the protein of claim 10, or the AAV particle of claim 11.

13. A plant comprising the plant cell of claim 12.

14. The plant cell of claim 12 or the plant of claim 13, wherein the plant cell or plant belong to the genera Nicotiana, Arabidopsis, Solanum, Cannabis, Fagopyrum, Oryza, or Zea.

15. The plant cell or plant of claim 14, wherein the plant is a Nicotiana species.

16. The plant cell or plant of claim 15, wherein the plant is Nicotiana benthamiana or Nicotiana tabacum.

17. Leaves, stems, flowers, or roots from any one of the plant cells or plants of claims 12-16.

18. A method for producing an AAV protein in a plant, comprising:

contacting a plant with Agrobacterium tumefaciens comprising at least one recombinant nucleic acid vector, wherein the at least one recombinant nucleic acid vector comprises a nucleic acid sequence that encodes an AAV protein and, wherein the nucleic acid sequences are codon optimized for expression in the plant, optionally using the recombinant nucleic acid vector of claim 9;

transferring the at least one recombinant nucleic acid vector to the cells of the plant;

expressing the AAV protein in the cells of the plant; and, optionally isolating the AAV protein from the cells of the plant.

19. The method of claim 18, wherein a plurality of AAV proteins are produced in the same plant.

20. The method of claim 19, wherein an AAV particle is produced in said plant and said AAV particle is, optionally, isolated from said plant.

21. The method of claim 20, wherein the AAV particle is capable of infecting a mammalian cell, optionally a human cell, optionally HEK293T.

22. The method of any one of claims 18-21, wherein the plant belongs to the genera Nicotiana, Arabidopsis, Solanum, Cannabis, Fagopyrum, Oryza, Lactuca or Zea.

23. The method of claim 22, wherein the plant is a Nicotiana species.

24. The method of claim 23, wherein the plant is Nicotiana benthamiana or Nicotiana tabacum and the nucleic acid sequences are codon optimized for expression in Nicotiana benthamiana or Nicotiana tabacum.

25. The method of any one of claims 18-24, wherein isolating the AAV protein comprises centrifugation, filtration and/or chromatography.

26. The method of claim 25, wherein the chromatography is affinity, ion exchange, anion exchange, size exclusion, or hydrophobic interaction chromatography.

27. The method of any one of claims 18-26, wherein the at least one recombinant nucleic acid vector comprises at least one sequence that has at least 90%, 91%, 92%, 93%, 94%, 95%, 96%, 97%, 98%, 99%, or 100% sequence identity to SEQ ID NOs: 2-11, 15-24, 28-37, or 40-49.

28. The method of any one of claims 18-27, wherein the plant yields at least 10⁷, 10⁸, 10⁹, 10¹⁰, 10¹¹, 10¹², 10¹³, or 10¹⁴ copies of the AAV protein.

29. The method of claim 28, wherein the plant yields at least 10¹², 10¹³, or 10¹⁴ copies of the AAV protein.

30. A method of gene therapy comprising administering an AAV particle produced and isolated by the method of any one of claims 18-29 to a cell of a subject in need thereof.

31. The recombinant nucleic acid vector of claim 9 or the AAV particle of claim 11 or the AAV particle produced by the method of claim 20 or 21 for use as a medicament.

32. The recombinant nucleic acid vector of claim 9 or the AAV particle of claim 11 or the AAV particle produced by the method of claim 20 or 21 for use in gene therapy to treat a human disease, such as inborn errors in metabolism, enzyme deficiencies, Pompe disease, Danon disease, neurodegenerative disorders, Parkinson's disease, Alzheimer's disease, motor neuron disease, muscular dystrophies, Duchenne muscular dystrophy, retinal degenerative disease, retinitis pigmentosa, Usher syndrome, Stargardt disease, or genetic causes of deafness.

33. A method of producing functional AAV particles in a plant, comprising:

transforming the plant with at least one recombinant nucleic acid vector comprising nucleic acid sequences that encode for components of the AAV particles or components that are involved in the assembly of the AAV particles;

growing the plant under conditions where the AAV particles are expressed and assembled in the plant; and

isolating the AAV particles from the plant.

34. The method of claim 33, wherein the step of transforming the plant is done by agroinfiltration.

35. The method of claim 33 or 34, wherein the nucleic acid sequence that encode for components of the AAV particles are codon optimized for the plant.

36. The method of any one of claims 33-35, wherein the plant belongs to the genera Nicotiana, Arabidopsis, Solanum, Cannabis, Fagopyrum, Oryza, Lactuca or Zea.

37. The method of any one of claims 33-36, wherein the plant is a Nicotiana, Lactuca, or Cannabis species.

38. The method of any one of claims 33-37, wherein the plant is Nicotiana benthamiana, Nicotiana tabacum, Lactuca sativa, or Cannabis sativa.

39. The method of any one of claims 33-38, wherein the components of the AAV particles or components that are involved in the assembly of the AAV particles comprise a REP protein, a CAP protein, an AAP protein, or an Ad5 E4orf6 protein, or any combination thereof.

40. The method of claim 39, wherein the REP protein is encoded by a nucleic acid sequence comprising a weak plant Kozak sequence that enhances translation of downstream in-frame polypeptides, and/or mutations in internal methionine codons to prevent potential expression of cryptic ORFs.

41. The method of claim 39 or 40, wherein the REP protein is encoded by a nucleic acid sequence having at least 90%, 91%, 92%, 93%, 94%, 95%, 96%, 97%, 98%, 99%, or 100% sequence identity to SEQ ID NOs: 1-11.

42. The method of any one of claims 39-41, wherein the REP protein comprises a peptide sequence having at least 90%, 91%, 92%, 93%, 94%, 95%, 96%, 97%, 98%, 99%, or 100% sequence identity to SEQ ID NO: 12 or 13.

43. The method of any one of claims 39-42, wherein the CAP protein is encoded by a nucleic acid sequence comprising a weak plant Kozak sequence that enhances translation of downstream in-frame polypeptides.

44. The method of any one of claims 39-43, wherein the CAP protein is encoded by a nucleic acid sequence having at least 90%, 91%, 92%, 93%, 94%, 95%, 96%, 97%, 98%, 99%, or 100% sequence identity to SEQ ID NOs: 14-24.

45. The method of any one of claims 39-44, wherein the CAP protein comprises a peptide sequence having at least 90%, 91%, 92%, 93%, 94%, 95%, 96%, 97%, 98%, 99%, or 100% sequence identity to SEQ ID NO: 25 or 26.

46. The method of any one of claims 39-45, wherein the AAP protein is encoded by a nucleic acid sequence having at least 90%, 91%, 92%, 93%, 94%, 95%, 96%, 97%, 98%, 99%, or 100% sequence identity to SEQ ID NOs: 27-37.

47. The method of any one of claims 39-46, wherein the AAP protein comprises a peptide sequence having at least 90%, 91%, 92%, 93%, 94%, 95%, 96%, 97%, 98%, 99%, or 100% sequence identity to SEQ ID NO: 38.

48. The method of any one of claims 39-47, wherein the Ad5 E4orf6 protein is encoded by a nucleic acid sequence having at least 90%, 91%, 92%, 93%, 94%, 95%, 96%, 97%, 98%, 99%, or 100% sequence identity to SEQ ID NOs: 39-49.

49. The method of any one of claims 39-48, wherein the Ad5 E4orf6 protein comprises a peptide sequence having at least 90%, 91%, 92%, 93%, 94%, 95%, 96%, 97%, 98%, 99%, or 100% sequence identity to SEQ ID NO: 50.

50. The method of any one of claims 33-49, wherein isolating the AAV particles comprises centrifugation, filtration and/or chromatography.

51. The method of claim 50, wherein the chromatography is affinity, ion exchange, anion exchange, size exclusion, or hydrophobic interaction chromatography.

52. The method of any one of claims 33-51, wherein at least 10⁷, 10⁸, 10⁹, 10¹⁰, 10¹¹, 10¹², 10¹³, or 10¹⁴ AAV particles are isolated from the plant.

53. The method of any one of claims 33-52, wherein at least 10¹², 10¹³, or 10¹⁴ AAV particles are isolated from the plant.

54. The method of any one of claims 33-53, wherein the AAV particles are capable of infecting a mammalian cell, optionally a human cell, optionally HEK293T.

55. The method of any one of claims 33-53, further comprising administering the AAV particles to a mammal, such as a human.

56. The AAV particles produced by the method of any one of claims 33-53 for use in the treatment of a disease.

57. The AAV particles produced by the method of any one of claims 33-53 for use in the manufacture of a medicament.

### BRIEF DESCRIPTION OF THE DRAWINGS

In addition to the features described above, additional features and variations will be readily apparent from the following descriptions of the drawings and exemplary embodiments. It is to be understood that these drawings depict typical embodiments and are not intended to be limiting in scope.

FIG. 1 depicts a sequence alignment of the AAV2 REP nucleic acid sequence codon optimized for N. benthamiana, A. thaliana, S. tuberosum, C. sativa, F. esculentum, O. sativa, Z. mays, S. lycopersicum, L. sativa and S. lycopersicoides. The sequence for N. benthamiana used in this alignment corresponds to the coding sequence of SEQ ID NO: 2. The sequence for A. thaliana used in this alignment corresponds to the coding sequence of SEQ ID NO: 3. The sequence for S. tuberosum used in this alignment corresponds to the coding sequence of SEQ ID NO: 4. The sequence for C. sativa used in this alignment corresponds to the coding sequence of SEQ ID NO: 5. The sequence for F. esculentum used in this alignment corresponds to the coding sequence of SEQ ID NO: 6. The sequence for O. sativa used in this alignment corresponds to the coding sequence of SEQ ID NO: 7. The sequence for Z. mays used in this alignment corresponds to the coding sequence of SEQ ID NO: 8. The sequence for S. lycopersicoides used in this alignment corresponds to the coding sequence of SEQ ID NO: 9. The sequence for S. lycopersicum used in this alignment corresponds to the coding sequence of SEQ ID NO: 10. The sequence for L. sativa used in this alignment corresponds to the coding sequence of SEQ ID NO: 11.

FIG. 2 depicts a sequence alignment of the AAV2 CAP nucleic acid sequence codon optimized for N. benthamiana, A. thaliana, S. tuberosum, C. sativa, F. esculentum, O. sativa, Z. mays, S. lycopersicum, L. sativa and S. lycopersicoides. The sequence for N. benthamiana used in this alignment corresponds to the coding sequence of SEQ ID NO: 15. The sequence for A. thaliana used in this alignment corresponds to the coding sequence of SEQ ID NO: 16. The sequence for S. tuberosum used in this alignment corresponds to the coding sequence of SEQ ID NO: 17. The sequence for C. sativa used in this alignment corresponds to the coding sequence of SEQ ID NO: 18. The sequence for F. esculentum used in this alignment corresponds to the coding sequence of SEQ ID NO: 19. The sequence for O. sativa used in this alignment corresponds to the coding sequence of SEQ ID NO: 20. The sequence for Z. mays used in this alignment corresponds to the coding sequence of SEQ ID NO: 21. The sequence for S. lycopersicoides used in this alignment corresponds to the coding sequence of SEQ ID NO: 22. The sequence for S. lycopersicum used in this alignment corresponds to the coding sequence of SEQ ID NO: 23. The sequence for L. sativa used in this alignment corresponds to the coding sequence of SEQ ID NO: 24.

FIG. 3 depicts a sequence alignment of the AAV2 AAP nucleic acid sequence codon optimized for N. benthamiana, A. thaliana, S. tuberosum, C. sativa, F. esculentum, O. sativa, Z. mays, S. lycopersicum, L. sativa, and S. lycopersicoides. The sequence for N. benthamiana used in this alignment corresponds to the coding sequence of SEQ ID NO: 28. The sequence for A. thaliana used in this alignment corresponds to the coding sequence of SEQ ID NO: 29. The sequence for S. tuberosum used in this alignment corresponds to the coding sequence of SEQ ID NO: 30. The sequence for C. sativa used in this alignment corresponds to the coding sequence of SEQ ID NO: 31. The sequence for F. esculentum used in this alignment corresponds to the coding sequence of SEQ ID NO: 32. The sequence for O. sativa used in this alignment corresponds to the coding sequence of SEQ ID NO: 33. The sequence for Z. mays used in this alignment corresponds to the coding sequence of SEQ ID NO: 34. The sequence for S. lycopersicoides used in this alignment corresponds to the coding sequence of SEQ ID NO: 35. The sequence for S. lycopersicum used in this alignment corresponds to the coding sequence of SEQ ID NO: 36. The sequence for L. sativa used in this alignment corresponds to the coding sequence of SEQ ID NO: 37.

FIG. 4 depicts a sequence alignment of the Ad5 E4orf6 nucleic acid sequence codon optimized for N. benthamiana, A. thaliana, S. tuberosum, C. sativa, F. esculentum, O. sativa, Z. mays, S. lycopersicum, L. sativa, and S. lycopersicoides. The sequence for N. benthamiana used in this alignment corresponds to the coding sequence of SEQ ID NO: 40. The sequence for A. thaliana used in this alignment corresponds to the coding sequence of SEQ ID NO: 41. The sequence for S. tuberosum used in this alignment corresponds to the coding sequence of SEQ ID NO: 42. The sequence for C. sativa used in this alignment corresponds to the coding sequence of SEQ ID NO: 43. The sequence for F. esculentum used in this alignment corresponds to the coding sequence of SEQ ID NO: 44. The sequence for O. sativa used in this alignment corresponds to the coding sequence of SEQ ID NO: 45. The sequence for Z. mays used in this alignment corresponds to the coding sequence of SEQ ID NO: 46. The sequence for S. lycopersicoides used in this alignment corresponds to the coding sequence of SEQ ID NO: 47. The sequence for S. lycopersicum used in this alignment corresponds to the coding sequence of SEQ ID NO: 48. The sequence for L. sativa used in this alignment corresponds to the coding sequence of SEQ ID NO: 49.

FIG. 5 depicts an experimental procedure for the production of AAV particles in plants using A. tumefaciens infiltration.

FIG. 6 depicts a plasmid map for pEAQ-HT-REPopt_AVGFPopt.

FIG. 7 depicts a plasmid map for pEAQ-HT-Ad5Orf6-OPT_AAV2-AAP-OPT.

FIG. 8 depicts a plasmid map for pEAQ-HT_CAPopt.

FIG. 9 depicts relative yields of AAV2 genomic particles in infiltrated N. benthamiana, N. tabacum, L. sativa, and C. sativa as detected by AAV2-specific qPCR.

FIG. 10A depicts a total protein-stained SDS-PAGE gel of N. benthamiana, L. sativa, and C. sativa leaf lysates showing the presence of bands corresponding to VP1, VP2, and VP3 protein.

FIG. 10B depicts a Western blot of N. benthamiana leaf lysate showing the presence of bands corresponding to VP1, VP2, and VP3 protein as detected by an anti-AAV2 VP monoclonal antibody. VP1=“*”, VP2=“\hat{ }”, VP3=“#”.

FIG. 11 depicts EGFP expression in HEK293T following transduction with plant produced AAV2-CMV-EGFP particles at an MOI of 2.7×10⁴, 2.7×10³, or 2.7×10² viral genomes per HEK293T cell.

FIG. 12 depicts exemplary sequences described in the present disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, technical and scientific terms used herein have the same meaning as commonly understood by one of ordinary skill in the art to which the present disclosure belongs. For purposes of the present disclosure, the following terms are defined below.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The practice of the present disclosure will employ, unless indicated specifically to the contrary, conventional methods of molecular biology and recombinant DNA techniques within the skill of the art.

The terms “function” and “functional” as used herein refer to a biological or enzymatic function.

The term “isolated” as used herein refers to material that is substantially or essentially free from components that normally accompany it in its native state. For example, an “isolated protein,” includes a protein that has been purified from the milieu or organism in its naturally occurring state.

The terms “nucleic acid” or “nucleic acid molecule” as used herein refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, adeno-associated virus (AAV), bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 300, 400, 500, 1000, 2000, 3000, 4000, or 5000 bases long, or any length in a range defined by any two of the aforementioned lengths. The term “downstream” on a nucleic acid as used herein refers to a sequence being after the 3′-end of a previous sequence, on the strand containing the encoding sequence (sense strand) if the nucleic acid is double stranded. The term “upstream” on a nucleic acid as used herein refers to a sequence being before the 5′-end of a subsequent sequence, on the strand containing the encoding sequence (sense strand) if the nucleic acid is double stranded. The term “grouped” on a nucleic acid as used herein refers to two or more sequences that occur in proximity either directly or with extra nucleic acids in between, e.g. linkers, repeats, or restriction enzyme sites, or any other sequence that is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 300, 400, 500, 1000, 2000, 3000, 4000, or 5000 bases long, or any length in a range defined by any two of the aforementioned lengths, but generally not with a sequence in between that encodes for a functioning or catalytic polypeptide, protein, or protein domain.

The term “codon optimized” regarding a nucleic acid as used herein refers to the substitution of codons of the nucleic acid to enhance or maximize translation in a host of a particular species without changing the polypeptide sequence based on species-specific codon usage biases and relative availability of each aminoacyl-tRNA in the target cell cytoplasm. Codon optimization and techniques to perform such optimization is known in the art. Additionally, synthetic codon optimized sequences can be obtained commercially from DNA sequencing services. Those skilled in the art will appreciate that gene expression levels are dependent on many factors, such as promoter sequences and regulatory elements. As noted for most bacteria, small subsets of codons are recognized by tRNA species leading to translational selection, which can be an important limit on protein expression. In this aspect, many synthetic genes can be designed to increase their protein expression level. In some embodiments, codon optimization of a gene for a certain organism results in a level of expression of the gene at least 100%, 150%, 200%, 250%, 300%, 350%, 400%, 450%, 500%, 550%, 600%, 650%, 700%, 750%, 800%, 850%, 900%, 950%, or 1000% of the level of expression with a non-codon optimized or wild type gene sequence.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

The terms “peptide”, “polypeptide”, and “protein” as used herein refers to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths.

In some embodiments, the nucleic acid or peptide sequences presented herein and used in the examples are optimized for plants but may also function in other organisms such as bacteria, fungi, protozoans, or animals. In other embodiments, nucleic acid or peptide sequences sharing 0%, 10%, 20%, 30%, 40%, 50%, 60%, 70%, 80%, 90%, 91%, 92%, 93%, 94%, 95%, 96%, 97%, 98%, 99%, or 100% similarity, or any percentage within a range defined by any two of the aforementioned percentages similarity to the nucleic acid or peptide sequences presented herein and used in the examples can also be used with no effect or little effect on the function of the sequences in biological systems. As used herein, the term “similarity” refers to a nucleic acid or peptide sequence having the same overall order of nucleotide or amino acids, respectively, as a template nucleic acid or peptide sequence with specific changes such as substitutions, deletions, repetitions, or insertions within the sequence. In some embodiments, two nucleic acid sequences sharing as low as 0%, 10%, 20%, 30%, 40%, 50%, 60%, 70%, 80%, 90%, 91%, 92%, 93%, 94%, 95%, 96%, 97%, 98%, or 99% similarity can encode for the same polypeptide by comprising different codons that encode for the same amino acid during translation.

As used herein, the terms “virion,” “virus or viral vector” and “viral particle” are interchangeably used, unless otherwise indicated.

As use herein, the term “packaging” refers to the events including production of single-strand viral genomes, assembly of coat (capsid) proteins, encapsulation of viral genomes, and the like. When an appropriate plasmid vector (normally, a plurality of plasmids) is introduced into a cell line that allows packaging under an appropriate condition, recombinant viral particles (i.e., virions, viral vectors) are constructed and secreted into the culture.

Viruses of the Parvoviridae family are small DNA animal viruses characterized by their ability to infect particular hosts, among other factors. Specifically, the family Parvoviridae is divided between two subfamilies: the Parvovirinae, which infect vertebrates, and the Densovirinae, which infect insects. The subfamily Parvovirinae (members of which are herein referred to as parvoviruses) includes the genus Dependovirus, which, under most conditions, require coinfection with a helper virus such as adenovirus, vaccinia virus, or herpes virus for productive infection in cell culture. Dependovirus includes adeno-associated virus (AAV), which normally infects humans (e.g. serotypes 2, 3A, 3B, 5, and 6) or primates (e.g. serotypes 1, 4, and rh10), and related viruses that infect other warm-blooded animals (e.g. bovine, canine, equine, and ovine adeno-associated viruses and bocaviruses).

In recent years, AAV has emerged as a preferred viral vector for gene therapy due to its ability to efficiently infect both non-dividing and dividing cells, maintain long term transgene expression from episomal non-integrating AAV genomes in mammalian cells, and pose relatively low pathogenic risk to humans. In view of these advantages, recombinant adeno-associated virus (rAAV) presently is being used in gene therapy clinical trials for neurological disorders, ophthalmologic disorders, hearing disorders, hemophilia B, malignant melanoma, cystic fibrosis, and other disease, and has recently passed FDA approval and BLA licensing for the treatment of the retinal degenerative disease Leber congenital amaurosis (LCA) and the motor neuron disease spino-muscular atrophy type 1 (SMA1).

AAV is able to infect a number of mammalian cells. Moreover, AAV transduction of human synovial fibroblasts is significantly more efficient than in similar murine cells, making AAV especially appealing for human gene therapy. Tropism of AAV differs significantly by serotype, underscoring the need to produce the AAV serotype most suitable for a particular target of gene therapy. rAAVs are currently produced in mammalian cells, including HEK 293T cells, COS cells, HeLa cells, KB cells, and other mammalian cell lines and in insect cells including Sf9, Sf21, and other insect cells using the baculovirus expression vector system (BEVS). See, e.g., U.S. Pat. Nos. 6,156,303, 5,387,484, 5,741,683, 5,691,176, and 5,688,676; U.S. PGPub 2002/0081721, and International Patent Applications WO 2000/047757, WO 2000/024916, WO 2003/042361, and WO 1996/017947, each of which is hereby expressly incorporated by reference in its entirety. The production of infectious AAV in non-mammalian, non-invertebrate plant cells and whole organisms is previously not known. The replication of parvoviral genomes including, particularly, Dependovirus genomes, in non-mammalian, non-invertebrate plant cells and whole organisms, is similarly previously not known.

Current methods for producing the large quantities of potent and high purity clinical grade AAV vectors rely on the use of mammalian cell culture or insect cell culture platforms. These platforms are expensive, non-standardized and non-modular, and are difficult to scale from the process and development scale to production scales required to meet a global demand for AAV gene therapy products, representing a significant bottleneck. According to J. Fraser Wright, “cGMP lots in the range of 10¹⁶ to 10¹⁸ viral genomes will be required to meet the requirements of late-stage clinical development and product licensure for many recombinant AAV products, especially those aimed at the most commercially viable disease applications” (J. F. Wright, “Adeno-associated viral vector manufacturing: keeping pace with accelerating clinical development,” Hum. Gene Ther., vol. 22, no. 8, pp. 913-914, August 2011, hereby expressly incorporated by reference in its entirety). AAV production using transient gene expression in plants would address the most significant challenges currently found with conventional mammalian and insect cell-based production methods. Namely, dramatically reduced production costs and infrastructure costs, modular production, scalable production, and a standardized production method and process for all AAV based viral vector products.

In the last 20 years, plants have become serious competitors to other production systems, such as bacteria, yeast, mammalian, or insect cells, for pharmaceuticals. Plants are robust, inexpensive to grow, and bring a low risk of contamination with endotoxins or mammalian pathogens which can be an issue with mammalian and insect cell cultures. Unlike prokaryotic expression systems, plants are able to introduce post-translational modification such as glycosylation. In insect and yeast cells, glycosylation is limited to very simple and inconsistent high mannose glycoforms. Any production system for pharmaceutical components, especially viral vectors, has to be quick in the response to a sudden increase in demand. Transient expression in plants can be adjusted rapidly with very low manufacturing costs that are linearly scalable with each individual plant representing a reproducible module of production and is highly efficient in terms of biomass production and viral vector yield. The advantages of transient plant bio-factories are the ease of manipulation, speed, low cost, and high protein yield per weight of plant tissue up to 1 g/Kg of biomass (Gleba et al., 2007; Thuenemann et al., 2013).

The difficulties involved in scaling-up rAAV production for clinical trials and commercialization using current mammalian and insect cell production systems can be significant, if not entirely prohibitive. For example, for certain clinical studies, more than 10¹⁵ particles of rAAV per dose may be required, meaning up to 10²⁰ particles per manufactured batch for large patient cohorts for a licensed drug. An example would be SRP9001 for the treatment of Duchenne muscular dystrophy from Sarepta Therapeutics, with patient dosing of 2×10¹⁴ vg/kg for children aged 3 months (average weight 6 kg) to 7 years (average weight 23 kg) old; and a global prevalence of ˜200,000 patients (Stark, A. E. Ann Transl Med. 2015 November; 3(19): 287 and clinical trial NCT03375164). Analysis of AAV production costs across clinical (200 L) or manufacturing (1000 L) scales calculated the inclusive cGMP production cost (Upstream, Downstream, QC, fill/finish) for 1×10¹⁴ vg of AAV using either adherent cell culture, single use bioreactors or fixed bed bioreactors ranging from $8000-$25000 (Cameau, E. et al. Cell Gene Therapy Insights 2019; 5(11), 1663-1675). This would make production costs for large scale cGMP global manufacturing for drug products such as SRP9001 prohibitively expensive using even optimized single use stirred or fixed bed mammalian cell bioreactors. Related difficulties associated with the production of AAV using known mammalian cell lines are recognized in the art. In addition, the insect cell BEVS system is subject to significant genome instability and genetic drift preventing effective development of stable producer cell lines. There is also the possibility that a vector destined for clinical use produced in a mammalian and insect cell culture will be contaminated with undesirable, perhaps pathogenic, material present in a mammalian or insect cell. In view of these and other issues, there remains a need for alternative and improved methods of efficiently, safely, and economically producing a large amount of infectious rAAV particles.

In contrast to cell culture-based production systems, plant biomass generation does not require the construction of expensive fermentation facilities, and correspondingly, scale-up production can be achieved without the need to construct duplicate facilities. As a result, plant biomass generation and upstream processing capacity can be operated and scaled in a capital-efficient manner with established agriculture practices. One 4-6 week old plantlet following infiltration/production and purification is estimated to be equivalent to one liter of suspension adapted mammalian cells based on experimentally determined yields of up to 1 g/kg of plant biomass for optimized recombinant protein production in N. benthamiana. By comparison, plant-made biologics cost significantly less than current cell culture-based systems because mammalian cell cultures require considerable startup investment and expensive growth media (Lai H, Chen Q Plant Cell Rep. 2012 March; 31(3):573-84). Plants also outpace the scalability of other expression systems, as recombinant protein-expressing biomass can be produced on an agricultural scale without the need to build duplicated bioreactors and associated facilities (Chen Q. Biological Engineering Transactions. 2008; 1:291-321). In contrast to bacterial cells, plants can produce large functional pharmaceutical proteins that require the proper post-translational modification of proteins, including glycosylation and the assembly of multiple hetero-subunits similar to mammalian or insect cells (Lai H, et al., Proc Natl Acad Sci USA. 2010 Feb. 9; 107(6):2419-24.)

Described herein are rapid, scalable, and cost-effective methods for producing clinical grade recombinant replication defective adeno-associated viral vectors in plants. Also disclosed herein are nucleic acid sequence that encode for AAV proteins and AAV genomes that have been codon optimized for efficient expression or function in plants.

AAV is a non-enveloped, replication defective virus around 20 nm in diameter with a single stranded DNA genome approximately 4.8 kilobases long. Over 100 serotypes of AAV have been identified, with at least 12 serotypes being characterized to some degree. These AAV serotypes exhibit remarkable divergence, such as the specific host cell receptor or primary receptor used for entry, and preference for certain host cell types (e.g. muscle cells, neurons, astrocytes, hepatocytes). For example, AAV1, 4, 5, and 6 bind to N- or O-linked sialylated proteoglycans, AAV9 binds to galactose, and AAV2 and 3 bind to heparin sulfate proteoglycans. AAV2 has historically been the best studied and utilized, but usage of different serotypes depending on their unique properties is possible. The AAV genome comprises three genes: REP, CAP, and AAP, but internal open reading frames and promoters in these genes result in multiple different proteins or protein fragments. REP encodes for REP78, REP68, REP52, and REP40, which are all involved in genome replication and packaging of viral particles. CAP encodes for VP1, VP2, and VP3, which form the icosahedral viral capsid. AAP, which is found within the CAP sequence in a different reading frame, encodes for the assembly-activating protein (AAP), which is needed for proper capsid formation at least in AAV2, but dispensable in other AAV serotypes. The nucleic acid material or genome that gets packaged into the AAV particles correspond to the sequence found flanked by inverted terminal repeats (ITR). In wild type viruses, the ITR flank the REP, CAP, and AAP gene sequences. For recombinant AAV, different transgenes, including but not limited to genes encoding an enzymatic marker (e.g. LacZ), genes encoding fluorescent proteins (e.g. GFP, EGFP), genes encoding optogenetic proteins (e.g. Chr2, ArctT, C1V1), genes encoding genetic sensors of cell metabolism, calcium and electrical activity (e.g. GCaMPs, rCaMPs, genetically encoded voltage sensors), genes encoding drug selection markers, genes encoding gene and RNA editing proteins (e.g. zinc finger nucleases, TALENs, CRISPR-Cas proteins, Streptococcus pyogenes Cas9, Streptococcus thermophilus Cas9, Staphylococcus aureus Cas9, Neisseria meningitidis Cas9, Francisella novicidia Cas12a or Cas12b, Prevotella sp. p 5-125 Cas13a, Cas13b, Cas13c or Cas13d, Porphyromonas gulae Cas13a, Cas13b, Cas13c, or Cas13d, Riemerella anatipestifer Cas13a, Cas13b, Cas13c, or Cas13d), genes to regulate or induce transgene expression (e.g. Dox inducible gene switches, Cumate inducible gene switches, PhyB-light regulated gene switches) or genes to treat a disease (e.g. CFTR for cystic fibrosis, Factor IX for hemophilia B, RPE65 for Leber congenital amaurosis, neurotrophins for neurodegenerative diseases). By excluding the REP proteins from the ITR-flanked region, the transgenes exist as episomes and can be expressed transiently by the host instead of integrating into the host genome. Hybrid AAV particles combining two or more serotypes can also be done to alter transducing efficiencies, cell type tropism, or affinity to host cell receptors.

As a replication defective virus, AAV requires a helper virus to replicate efficiently. Co-infection with an adenovirus accomplishes this but leads to adenoviral contamination during purification. To avoid this, expression (either from the nucleic acid vector containing the AAV genes, or previously engineering the host cell) of the E1, E2A, E4 and VA regions of the adenovirus genome provides an additional set of components needed for efficient AAV production. In some embodiments, the E1, E2A, and VA regions are only needed for efficient AAV production when using an endogenous AAV promoter. In some embodiments, the AAV genes can be driven with other promoters, such as constitutive promoters, inducible promoters, other viral promoters, mammalian promoters, bacterial promoters, fungal promoters, or plant promoters. In some embodiments, only the E4 region is needed for AAV replication. In some embodiments, the adenovirus type 5 E4orf6 gene (Ad5 E4orf6) is provided with the AAV expression vectors during transformation of the plant to increase AAV yield.

In some embodiments, AAV particles are produced under sterile conditions and under regulated or controlled procedures. Methods for maintaining and ensuring sterility may adhere to good manufacturing practice (GMP), good tissue practice (GTP), good laboratory practice (GLP), and good distribution practice (GDP) standards. Methods for maintaining and ensuring sterility include but are not limited to high-efficiency particulate air (HEPA) filtration, wet or dry heat, radiation, e.g., X-rays, gamma rays, or UV light, sterilizing agents or fumigants, such as ethylene oxide, nitrogen dioxide, ozone, glutaraldehyde, formaldehyde, peracetic acid, chlorine dioxide, or hydrogen peroxide, aseptic filling of sterile containers, packaging in plastic film or wrap, or vacuum sealing.

AAV are purified with methods to provide optimal yield of functional viral particles while excluding potential contaminants that may harm individuals and avoiding purification of non-functional empty capsids. Toward this goal, AAV can be purified using techniques known in the art, including but not limited to extraction, freeze-thawing, homogenization, permeabilization, centrifugation, density gradient centrifugation, CsCl gradient centrifugation, iodixanol gradient centrifugation, ultracentrifugation, fractionation, precipitation, SDS-PAGE, native PAGE, size exclusion chromatography, liquid chromatography, gas chromatography, hydrophobic interaction chromatography, ion exchange chromatography, anion exchange chromatography, cation exchange chromatography, affinity chromatography, heparin sulfate affinity chromatography, sialic acid affinity chromatography, immunoaffinity chromatography, metal binding chromatography, nickel column chromatography, epitope tag purification, or lyophilization, or any combination thereof.

As with any other group of organisms, certain plants have found favor for use in research or production due to properties such as size, growth rate, ease of culture, available pathogens or vectors, disease resistance, adaptability to external conditions, light requirements, ease of genetic manipulation, types of phytochemicals produced, or availability of a genomic sequence. Plants that are useful for these properties or any other desirable property include but are not limited to Nicotiana, Nicotiana benthamiana, Nicotiana tabacum, Arabidopsis, Arabidopsis thaliana, Solanum, Solanum tuberosum, Solanum lycopersicum, Solanum lycopersicoides, Cannabis, Cannabis sativa, Fagopyrum, Fagopyrum esculentum, Oryza, Oryza sativa, Zea, Zea mays, Hordeum, Hordeum vulgare, Selaginella, Selaginella moellendorffii, Brachypodium, Brachypodium distachyon, Lotus, Lotus japonicus, Lemna, Lemna gibba, Medicago, Medicago truncatula, Mimulus, Mimulus guttatus, Physcomitrella, Physcomitrella patens, Populus, Populus trichocarpa, Lactuca, Lactuca sativa, or any plant species able to be transformed by Agrobacterium tumefaciens. In some embodiments, the plant belongs to Nicotiana. In some preferred embodiments, the plant is Nicotiana benthamiana.

Agrobacterium tumefaciens is a bacterium pathogenic to plants, causing galls, crown galls, or tumors in the plant. A. tumefaciens accomplishes this through the tumor inducing plasmid (Ti plasmid), which comprises a T-DNA region which gets transferred to the host plant and a pathogenicity island or virulence region of genes encoding a type IV secretion mechanism used to perform said transfer. The T-DNA region comprises genes encoding proteins that synthesize plant hormones such as auxin and cytokinin, which cause growth of the galls or tumors. By removing these genes (to abolish formation of the disease) and inserting desirable genes for expression, A. tumefaciens is a potent tool for genetically engineering plants. Successful transformation of A. tumefaciens or plants may be selected by, for example, resistance to neomycin, kanamycin, or G418 (geniticin) through expression of neomycin phosphotransferase. More information about transformation of plants using A. tumefaciens can be found in U.S. Pat. No. 5,792,935, hereby expressly incorporated by reference in its entirety.

As used herein, “plant promoter” refers to the untranslated nucleic acid sequence upstream of a coding sequence that initiate transcription. Plants can have promoters responsive to certain environmental conditions, including but not limited to light responsive promoters, stress responsive promoters, plant hormone responsive promoters, sucrose responsive promoters, low-oxygen responsive promoters, or the nopaline synthase promoter. For the production and subsequent purification of AAV and other viruses or proteins in plants, strong constitutive promoters are typically desired. In some embodiments, some strong constitutive promoters used include but are not limited to Cauliflower Mosaic Virus 35S promoter, Cowpea Mosaic Virus promoter, opine promoters, ubiquitin promoters, rice actin 1 promoter, or maize alcohol dehydrogenase 1 promoter. In some embodiments, a pEAQ-HT vector is used to transform plants either transiently or stably using A. tumefaciens (agroinfiltration). This pEAQ vector uses the Cowpea Mosaic Virus promoter sequence (with a U162C mutation to enhance activity) within the T-DNA to obtain high rates of protein expression without extraneous virus production in plants. However, in other embodiments, different plant expression vectors such as pBINPLUS, pPZP3425, pPZP5025, pPZPTRBO, pJLTRBO, or pBY030-2R can be used. More information about the pEAQ vectors is provided in U.S. Pat. No. 8,674,084, hereby expressly incorporated by reference in its entirety.

As used herein, the term “plantlet” refers to young plants. Relative to fully grown plants, plantlets are smaller, therefore easier to handle, and experience rapid growth and cellular activity. In some embodiments, small scale purification of AAV involves the use of at least 1, 2, 3, 4, 5, 6, 7, 8, 9, 10, 15, 20, 25, 30, 35, 40, 45, 50, 60, 70, 80, 90, or 100 plantlets. In other embodiments, larger scale purification of AAV can be scaled up to use at least 100, 200, 300, 400, 500, 600, 700, 800, 900, 1000, 2000, 5000, 10000, 20000, 30000, 40000, or 50000 plants.

The term “purity” of any given substance, compound, or material as used herein refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to nucleic acids, DNA, RNA, nucleotides, proteins, polypeptides, peptides, amino acids, lipids, cell membrane, cell debris, small molecules, degradation products, solvent, carrier, vehicle, or contaminants, or any combination thereof. In some embodiments, the AAV product is substantially free of host cell proteins, host cell nucleic acids, plasmid DNA, empty viral vectors, AAV particles with incomplete protein composition and oligomerized structures, or contaminating viruses, e.g., non AAV, lipid enveloped viruses, Heat shock protein 70 (HSP70), Lactate dehydrogenase (LDH), proteasomes, contaminant non-AAV viruses, host cell culture components, process related components, mycoplasma, pyrogens, bacterial endotoxins, and adventitious agents. Purity can be measured using technologies including but not limited to electrophoresis, SDS-PAGE, capillary electrophoresis, PCR, rtPCR, qPCR, chromatography, liquid chromatography, gas chromatography, thin layer chromatography, enzyme-linked immunosorbent assay (ELISA), spectroscopy, UV-visible spectrometry, infrared spectrometry, mass spectrometry, nuclear magnetic resonance, gravimetry, or titration, or any combination thereof.

Producing AAV particles in plant or plant material using techniques such as agroinfiltration results in greater purity of AAV compared to techniques known in the art, such as production in mammalian or insect cells. In some embodiments, plant-derived AAV particles are free of animal or mammalian cellular components, animal or mammalian-specific pathogens, including viruses, bacteria, protozoans, and fungi, serum, bovine serum, antibiotics, or hormones, or any combination thereof.

The term “yield” of any given substance, compound, or material as used herein refers to the actual overall amount of the substance, compound, or material relative to the expected overall amount. For example, the yield of the substance, compound, or material may be at least 80, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

Producing AAV particles in plant or plant material using techniques such as agroinfiltration results in greater yield of AAV compared to techniques known in the art, such as production in mammalian or insect cells. In some embodiments, one 4-6 week old plantlet yields at least 10⁷, 10⁸, 10⁹, 10¹⁰, 10¹¹, 10¹², 10¹³, or 10¹⁴ AAV particles.

The invention is generally disclosed herein using affirmative language to describe the numerous embodiments. The invention also includes embodiments in which subject matter is excluded, in full or in part, such as substances or materials, method steps and conditions, protocols, or procedures.

### AAV Particles and Components

Disclosed herein in some embodiments are nucleic acid molecules comprising a sequence that encodes an AAV2 REP protein. In some embodiments, the REP protein comprises REP78, REP68, REP52, or REP 40. In some embodiments, the sequence has at least 90%, 91%, 92%, 93%, 94%, 95%, 96%, 97%, 98%, 99%, or 100% sequence identity to SEQ ID NO: 2-11. In some embodiments, the sequence has at least 90%, 91%, 92%, 93%, 94%, 95%, 96%, 97%, 98%, 99%, or 100% sequence identity to SEQ ID NO: 2

Also disclosed herein in some embodiments are nucleic acid molecules comprising a sequence that encodes an AAV2 CAP protein. In some embodiments, the CAP protein comprises VP1, VP2, or VP3. In some embodiments, the sequence has at least 90%, 91%, 92%, 93%, 94%, 95%, 96%, 97%, 98%, 99%, or 100% sequence identity to SEQ ID NO: 15-24. In some embodiments, the sequence has at least 90%, 91%, 92%, 93%, 94%, 95%, 96%, 97%, 98%, 99%, or 100% sequence identity to SEQ ID NO: 15.

Also disclosed herein in some embodiments are nucleic acid molecules comprising a sequence that encodes an AAV2 AAP protein. In some embodiments, the sequence has at least 90%, 91%, 92%, 93%, 94%, 95%, 96%, 97%, 98%, 99%, or 100% sequence identity to SEQ ID NO: 28-37. In some embodiments, the sequence has at least 90%, 91%, 92%, 93%, 94%, 95%, 96%, 97%, 98%, 99%, or 100% sequence identity to SEQ ID NO: 28.

Also disclosed herein in some embodiments are nucleic acid molecules comprising a sequence that encodes an Ad5 E4orf6 protein. In some embodiments, the sequence has at least 90%, 91%, 92%, 93%, 94%, 95%, 96%, 97%, 98%, 99%, or 100% sequence identity to SEQ ID NO: 40-49. In some embodiments, the sequence has at least 90%, 91%, 92%, 93%, 94%, 95%, 96%, 97%, 98%, 99%, or 100% sequence identity to SEQ ID NO: 40.

Also disclosed herein in some embodiments are recombinant nucleic acid vectors comprising any one or more of the nucleic acid molecules disclosed herein. Also disclosed herein in some embodiments are proteins encoded by any one of the nucleic acid molecules or nucleic acid vectors disclosed herein. Also disclosed herein in some embodiments are AAV particles comprising any one or more of the nucleic acid molecules, nucleic acid vectors, or proteins disclosed herein.

Also disclosed herein in some embodiments are plant cells comprising any one or more of the nucleic acid molecules, nucleic acid vectors, proteins, or AAV particles disclosed herein. Also disclosed herein in some embodiments are plants comprising any one of the plant cells disclosed herein. In some embodiments, the plant cell or plant belong to the genera Nicotiana, Arabidopsis, Solanum, Cannabis, Fagopyrum, Oryza, or Zea. In some embodiments, the plant is a Nicotiana species. In some embodiments, the plant is Nicotiana benthamiana or Nicotiana tabacum.

Also disclosed herein in some embodiments are leaves, stems, flowers, or roots from any one of the plant cells or plants disclosed herein.

### Methods of Making and Use

Disclosed herein are methods for producing an AAV protein in a plant. In some embodiments, the methods comprise contacting a plant with Agrobacterium tumefaciens comprising at least one recombinant nucleic acid vector, transferring the at least one recombinant nucleic acid vector to the cells of the plant, expressing the AAV protein in the cells of the plant, and, optionally, isolating the AAV protein from the cells of the plant. In some embodiments, the at least one recombinant nucleic acid vector comprises a nucleic acid sequence that encodes an AAV protein. In some embodiments, the nucleic acid sequence are codon optimized for expression in the plant. In some embodiments, the nucleic acid sequences are part of any one of the nucleic acid vectors disclosed herein. In some embodiments, a plurality of AAV proteins are produced in the same plant. In some embodiments, an AAV particle is produced in the plant and the AAV particle is, optionally, isolated from the plant. In some embodiments, the AAV particle is capable of infecting a mammalian cell, optionally a human cell, optionally HEK293T. In some embodiments, the plant belongs to the genera Nicotiana, Arabidopsis, Solanum, Cannabis, Fagopyrum, Oryza, Lactuca or Zea. In some embodiments, the plant is a Nicotiana species. In some embodiments, the plant is Nicotiana benthamiana or Nicotiana tabacum and the nucleic acid sequences are codon optimized for expression in Nicotiana benthamiana or Nicotiana tabacum. In some embodiments, the plant is a Lactuca species. In some embodiments, the plant is Lactuca sativa and the nucleic acid sequences are codon optimized for expression in Lactuca sativa. In some embodiments, the plant is a Cannabis species. In some embodiments, the plant is Cannabis sativa and the nucleic acid sequences are codon optimized for expression in Cannabis sativa. In some embodiments, isolating the AAV protein comprises centrifugation, filtration and/or chromatography. In some embodiments, the chromatography is affinity, ion exchange, anion exchange, size exclusion, or hydrophobic interaction chromatography. In some embodiments, the at least one recombinant nucleic acid vector comprises at least one sequence that has at least 90%, 91%, 92%, 93%, 94%, 95%, 96%, 97%, 98%, 99%, or 100% sequence identity to SEQ ID NOs: 2-11, 15-24, 28-37, or 40-49. In some embodiments, the plant yields at least 10⁴, 10⁵, 10⁶, 10⁷, 10⁸, 10⁹, 10¹⁰, 10¹¹, 10¹², 10¹³, or 10¹⁴ copies of the AAV protein. In some embodiments, the plant yields at least 10¹², 10¹³, or 10¹⁴ copies of the AAV protein.

Also disclosed herein are methods of producing functional AAV particles in a plant. In some embodiments, the methods comprise transforming the plant with at least one recombinant nucleic acid vector comprising nucleic acid sequences that encode for components of the AAV particles or components that are involved in the assembly of the AAV particles, growing the plant under conditions where the AAV particles are expressed and assembled in the plant, and isolating the AAV particles from the plant. In some embodiments, the step of transforming the plant is done by agroinfiltration. In some embodiments, the nucleic acid sequence that encode for components of the AAV particles are codon optimized for the plant. In some embodiments, the plant belongs to the genera Nicotiana, Arabidopsis, Solanum, Cannabis, Fagopyrum, Oryza, Lactuca or Zea. In some embodiments, the plant is a Nicotiana, Lactuca, or Cannabis species. In some embodiments, the plant is Nicotiana benthamiana, Nicotiana tabacum, Lactuca sativa, or Cannabis sativa. In some embodiments, the components of the AAV particles or components that are involved in the assembly of the AAV particles comprise a REP protein, a CAP protein, an AAP protein, or an Ad5 E4orf6 protein, or any combination thereof.

In any of the methods disclosed herein, in some embodiments, the REP protein is encoded by a nucleic acid sequence comprising a weak plant Kozak sequence that enhances translation of downstream in-frame polypeptides, and/or mutations in internal methionine codons to prevent potential expression of cryptic ORFs. In some embodiments, the REP protein is encoded by a nucleic acid sequence having at least 90%, 91%, 92%, 93%, 94%, 95%, 96%, 97%, 98%, 99%, or 100% sequence identity to SEQ ID NOs: 1-11. In some embodiments, the REP protein comprises a peptide sequence having at least 90%, 91%, 92%, 93%, 94%, 95%, 96%, 97%, 98%, 99%, or 100% sequence identity to SEQ ID NO: 12 or 13.

In any of the methods disclosed herein, in some embodiments, the CAP protein is encoded by a nucleic acid sequence comprising a weak plant Kozak sequence that enhances translation of downstream in-frame polypeptides. In some embodiments, the CAP protein is encoded by a nucleic acid sequence having at least 90%, 91%, 92%, 93%, 94%, 95%, 96%, 97%, 98%, 99%, or 100% sequence identity to SEQ ID NOs: 14-24. In some embodiments, the CAP protein comprises a peptide sequence having at least 90%, 91%, 92%, 93%, 94%, 95%, 96%, 97%, 98%, 99%, or 100% sequence identity to SEQ ID NO: 25 or 26.

In any of the methods disclosed herein, in some embodiments, the AAP protein is encoded by a nucleic acid sequence having at least 90%, 91%, 92%, 93%, 94%, 95%, 96%, 97%, 98%, 99%, or 100% sequence identity to SEQ ID NOs: 27-37. In some embodiments, the AAP protein comprises a peptide sequence having at least 90%, 91%, 92%, 93%, 94%, 95%, 96%, 97%, 98%, 99%, or 100% sequence identity to SEQ ID NO: 38. In some embodiments, the Ad5 E4orf6 protein is encoded by a nucleic acid sequence having at least 90%, 91%, 92%, 93%, 94%, 95%, 96%, 97%, 98%, 99%, or 100% sequence identity to SEQ ID NOs: 39-49. In some embodiments, the Ad5 E4orf6 protein comprises a peptide sequence having at least 90%, 91%, 92%, 93%, 94%, 95%, 96%, 97%, 98%, 99%, or 100% sequence identity to SEQ ID NO: 50.

In any of the methods disclosed herein, isolating the AAV particles comprises centrifugation, filtration, and/or chromatography. In some embodiments, the chromatography is affinity, ion exchange, anion exchange, size exclusion, or hydrophobic interaction chromatography. In some embodiments, at least 10⁴, 10⁵, 10⁶, 10⁷, 10⁸, 10⁹, 10¹⁰, 10¹¹, 10¹², 10¹³, or 10¹⁴ AAV particles are isolated from the plant. In some embodiments, at least 10¹², 10¹³, or 10¹⁴ AAV particles are isolated from the plant. In some embodiments, the AAV particles are capable of infecting a mammalian cell, optionally a human cell, optionally HEK293T.

In any of the methods disclosed herein, the methods further comprise administering the AAV particles to a mammal. In some embodiments, the mammal is a human.

Also disclosed herein are methods of gene therapy. In some embodiments, the methods comprise administering an AAV particle produced and isolated by any one of the methods disclosed herein to a cell of a subject in need thereof.

Also disclosed herein are the recombinant nucleic acid vectors or AAV particles disclosed herein for use as a medicament.

Also disclosed herein are the recombinant nucleic acid vectors or AAV particles disclosed herein for use in gene therapy to treat a human disease. In some embodiments, the human diseases is inborn errors in metabolism, enzyme deficiencies, Pompe disease, Danon disease, neurodegenerative disorders, Parkinson's disease, Alzheimer's disease, motor neuron disease, muscular dystrophies, Duchenne muscular dystrophy, retinal degenerative disease, retinitis pigmentosa, Usher syndrome, Stargardt disease, or genetic causes of deafness.

Also disclosed herein are the AAV particles produced by any of the methods disclosed herein for use in the treatment of a disease.

Also disclosed herein are the AAV particles produced by any of the methods disclosed herein for use in the manufacture of a medicament.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Example 1: AAV Sequences

Wild-type nucleic acid sequences of AAV2 REP, CAP, and AAP and Ad5 E4orf6 were codon optimized for expression in several plants, including but not limited to Nicotiana benthamiana, Nicotiana tabacum, Arabidopsis thaliana, Solanum tuberosum, Cannabis sativa, Fagopyrum esculentum, Oryza sativa, Zea mays, Solanum lycopersicoides, Solanum lycopersicum, or Lactuca sativa. These nucleic acid sequences are represented in Table 1. Corresponding translated protein sequences are represented in Table 2.

TABLE 1
Nucleic acid sequences of viral components

| Organism | AAV2 REP | AAV2 CAP | AAV2 AAP | Ad5 E4orf6 |
|---|---|---|---|---|
| AAV2 Wild-type | SEQ ID NO: 1 | SEQ ID NO: 14 | SEQ ID NO: 27 | SEQ ID NO: 39 |
| N. benthamiana, | SEQ ID NO: 2 | SEQ ID NO: 15 | SEQ ID NO: 28 | SEQ ID NO: 40 |
| N. tabacum | | | | |
| A. thaliana | SEQ ID NO: 3 | SEQ ID NO: 16 | SEQ ID NO: 29 | SEQ ID NO: 41 |
| S. tuberosum | SEQ ID NO: 4 | SEQ ID NO: 17 | SEQ ID NO: 30 | SEQ ID NO: 42 |
| C. sativa | SEQ ID NO: 5 | SEQ ID NO: 18 | SEQ ID NO: 31 | SEQ ID NO: 43 |
| F. esculentum | SEQ ID NO: 6 | SEQ ID NO: 19 | SEQ ID NO: 32 | SEQ ID NO: 44 |
| O. sativa | SEQ ID NO: 7 | SEQ ID NO: 20 | SEQ ID NO: 33 | SEQ ID NO: 45 |
| Z. mays | SEQ ID NO: 8 | SEQ ID NO: 21 | SEQ ID NO: 34 | SEQ ID NO: 46 |
| S. lycopersicoides | SEQ ID NO: 9 | SEQ ID NO: 22 | SEQ ID NO: 35 | SEQ ID NO: 47 |
| S. lycopersicum | SEQ ID NO: 10 | SEQ ID NO: 23 | SEQ ID NO: 36 | SEQ ID NO: 48 |
| L. sativa | SEQ ID NO: 11 | SEQ ID NO: 24 | SEQ ID NO: 37 | SEQ ID NO: 49 |

TABLE 2
Protein sequences of viral components

| Organism | AAV2 REP | AAV2 CAP | AAV2 AAP | Ad5 E4orf6 |
|---|---|---|---|---|
| AAV2 Wild-type | SEQ ID NO: 12 | SEQ ID NO: 25 | SEQ ID NO: 38 | SEQ ID NO: 50 |
| N. benthamiana | SEQ ID NO: 13 | SEQ ID NO: 26 | SEQ ID NO: 38 | SEQ ID NO: 50 |
| N. tabacum | | | | |
| A. thaliana | | | | |
| S. tuberosum | | | | |
| C. sativa | | | | |
| F. esculentum | | | | |
| O. sativa | | | | |
| Z. mays | | | | |
| S. lycopersicoides | | | | |
| S. lycopersicum | | | | |
| L. sativa | | | | |

The nucleic acid sequences for all plant codon optimized cDNA sequences for REP (SEQ ID NOs: 2-11) and CAP (SEQ ID NOs: 15-24) as shown herein have been engineered with nucleotide differences compared to the sequences for wild-type (SEQ ID NOs: 1 and 14). The modified REP sequences begin with the sequence GGGTTTATGACTGGT (SEQ ID NO: 54), which forms a weak plant Kozak sequence that enhances translation of the downstream in-frame polypeptides (i.e. REP52), and the modified CAP sequences begin with the sequence GGGTTTATGACTGGCCGCCGGTTAT (SEQ ID NO: 55), which forms a weak plant Kozak sequence that enhances translation of the downstream in-frame polypeptides (i.e. VP2, VP3). Wild-type REP translates to SEQ ID NO: 12, and wild-type CAP translates to SEQ ID NO: 25. Plant codon optimized REP translates to SEQ ID NO: 13, and plant codon optimized CAP translates to SEQ ID NO: 25. The plant codon optimized proteins AAP (SEQ ID NO: 38) and E4orf6 (SEQ ID NO: 50) are unchanged from wild-type.

The plant codon optimized sequences for REP have been modified to enhance expression or ratio of expression of the four in-frame proteins, REP78, REP68, REP52, and REP40. Codon 2 (CCG, proline) were substituted (to ACT, threonine) to create a weak Kozak sequence, increasing the expression rate of REP52 and REP40, which initiate with an internal start codon by leaky mRNA ribosome scanning. In addition, internal methionine residues (M43, M91, M103, and M172) were mutated to leucine to eliminate in frame start codons between the REP78 and REP52 ATG start codons, preventing potential expression of cryptic ORFs. REP52 and REP40 initiate at codon 225. It is envisioned that any one or more of these mutations are optional.

Similarly, the plant codon optimized sequences for CAP have been modified to enhance expression or ratio of expression of the three in-frame proteins, VP1, VP2, and VP3. The first 6 amino acids of CAP (corresponding to the first 6 amino acids of VP1), of the wild-type sequence is MAADGY. For the plant codon optimized sequences, these amino acids were changed to MTAAGY to create a weak Kozak sequence, increasing the expression rate of VP2 and VP3, which initiate with internal start codons by leaky mRNA ribosome scanning. VP2 initiates with the alternative start codon ACG at codon 138, and VP3 initiates with ATG at codon 203. It is envisioned that any one or more of these mutations are optional.

Although these nucleic acid and amino acid changes to REP and CAP to improve AAV production in plants is exemplified with N. benthamiana, they are also applied to the other plants listed herein, or any other genetically tractable plant, with no anticipated issues or limitations, as embodied in the codon optimized and transcriptionally optimized cDNA and protein sequences for N. benthamiana, N. tabacum, A. thaliana, S. tuberosum, C. sativa, F. esculentum, O. sativa, Z. mays, S. lycopersicoides, S. lycopersicum, and L. sativa.

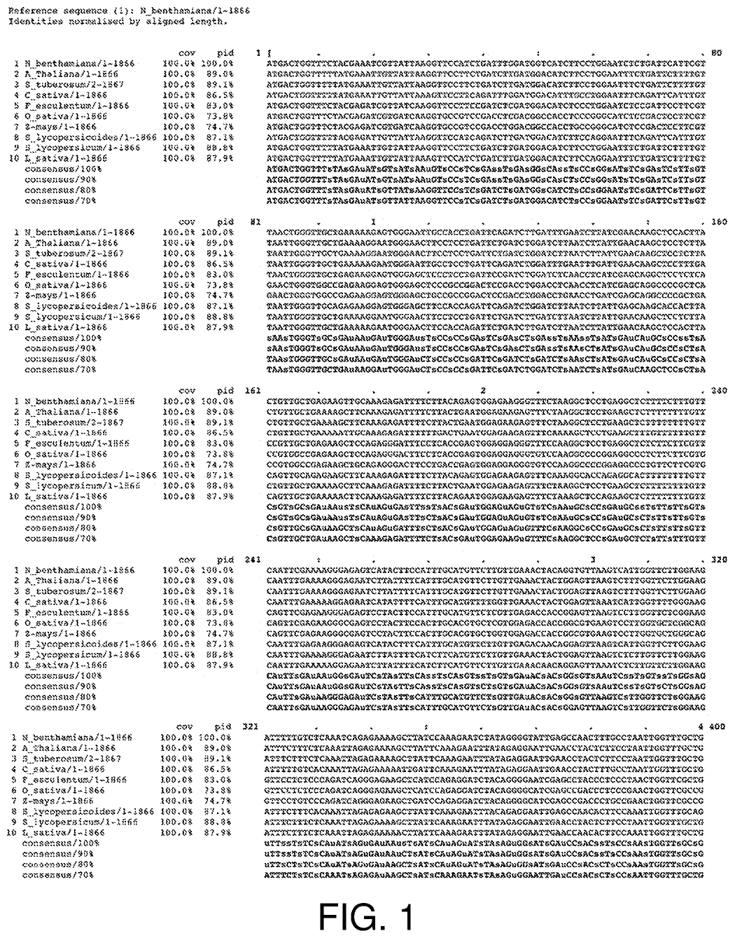

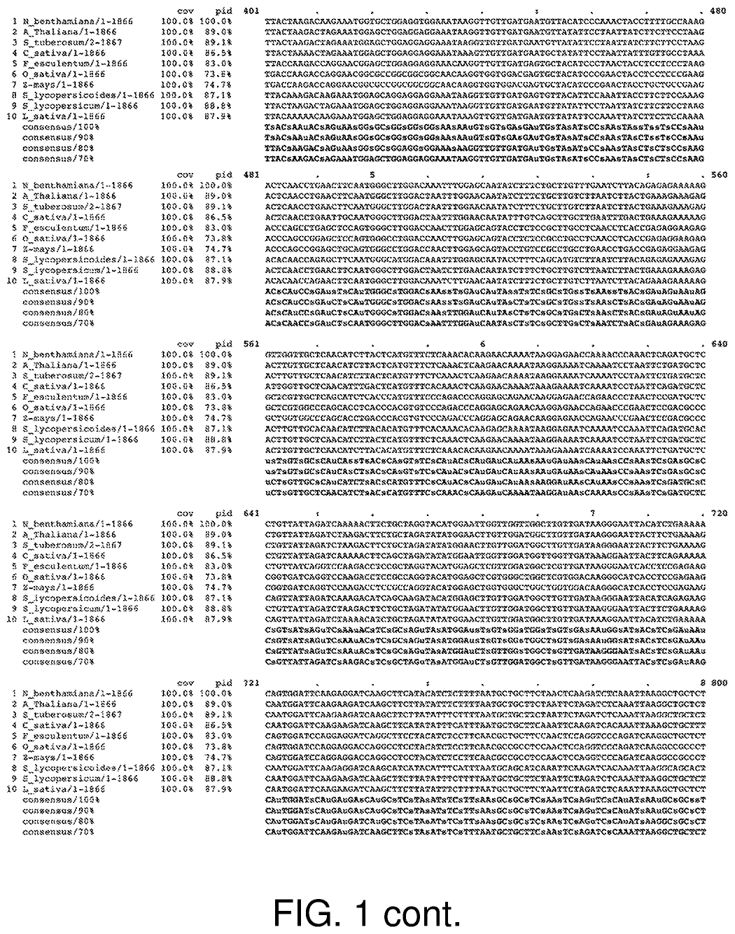

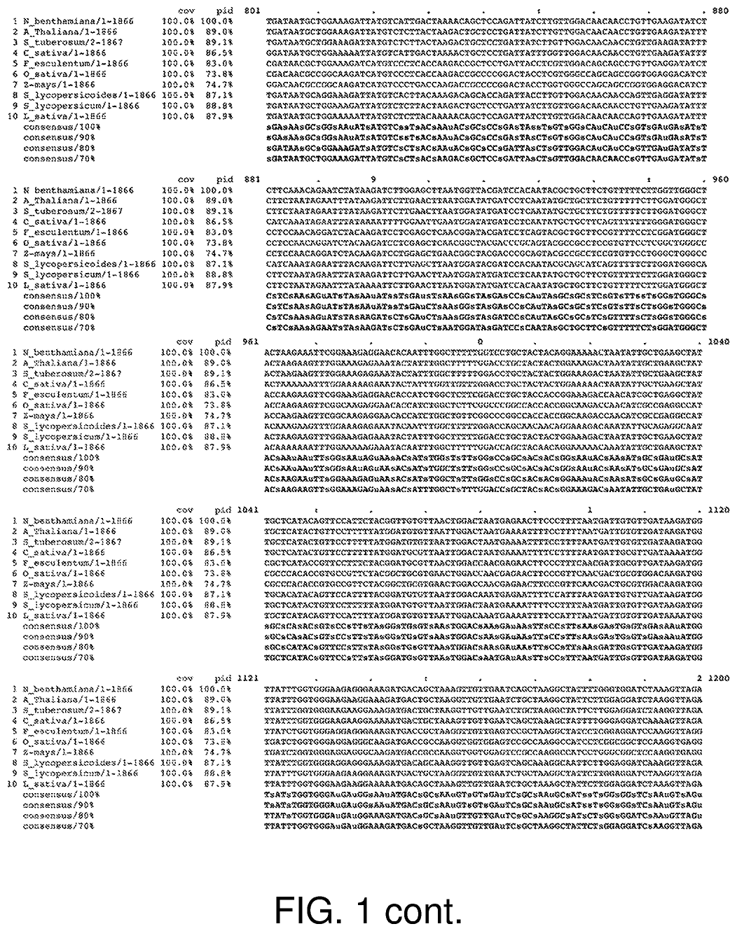

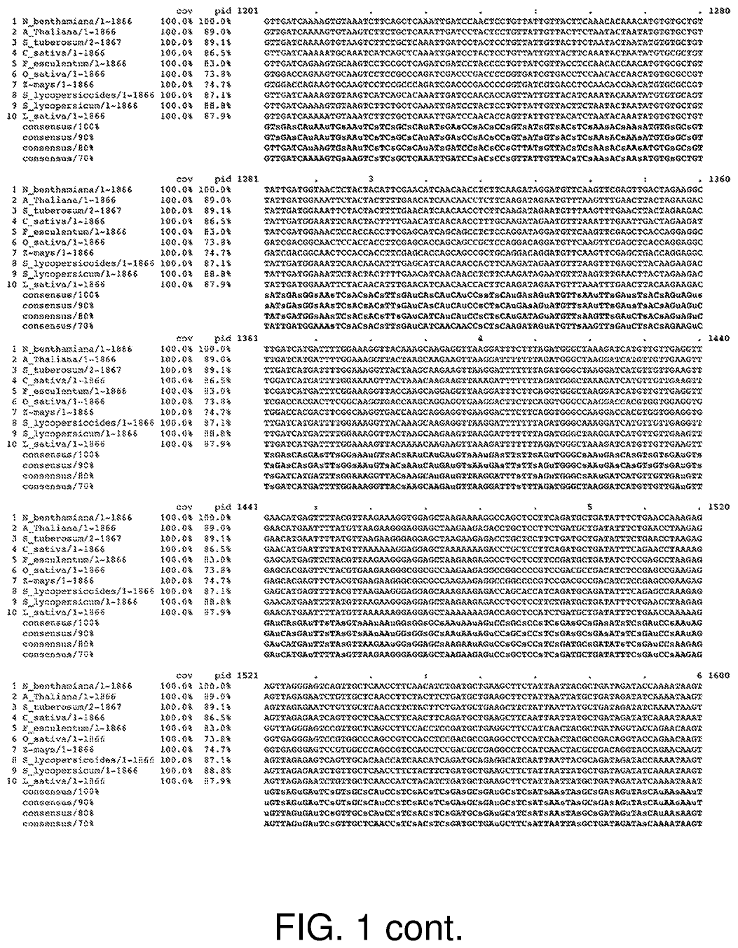

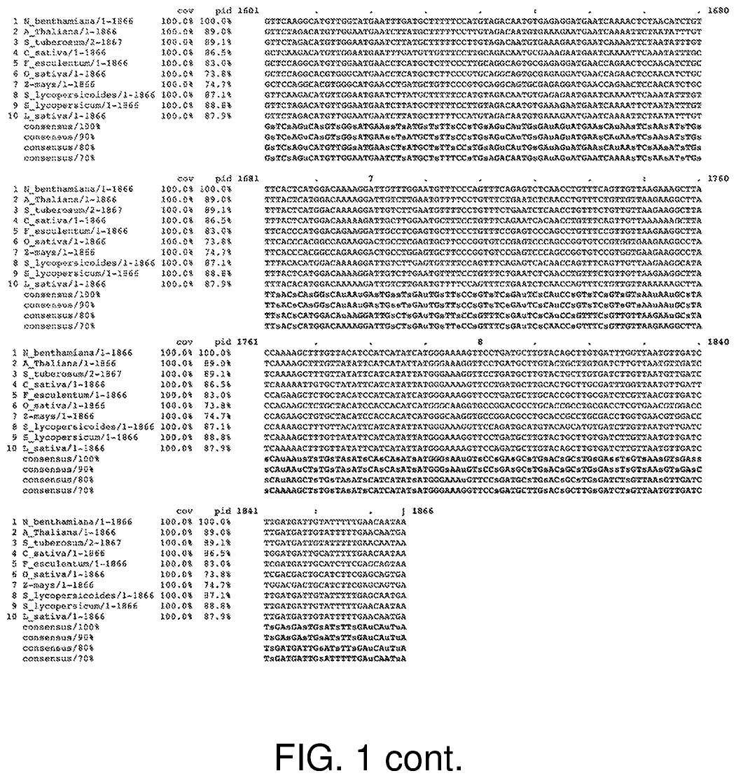

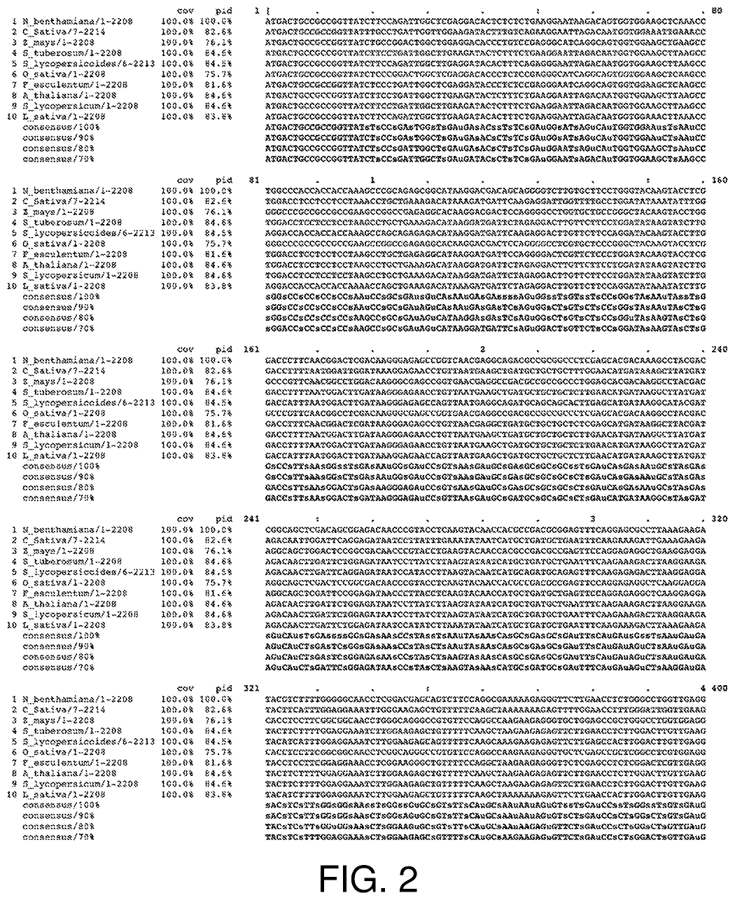

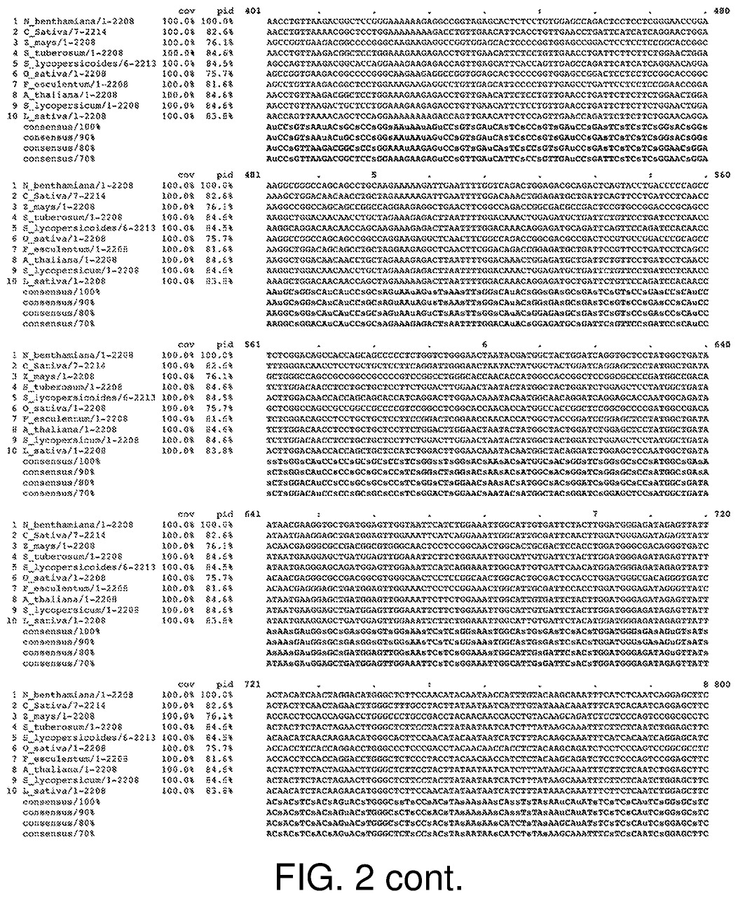

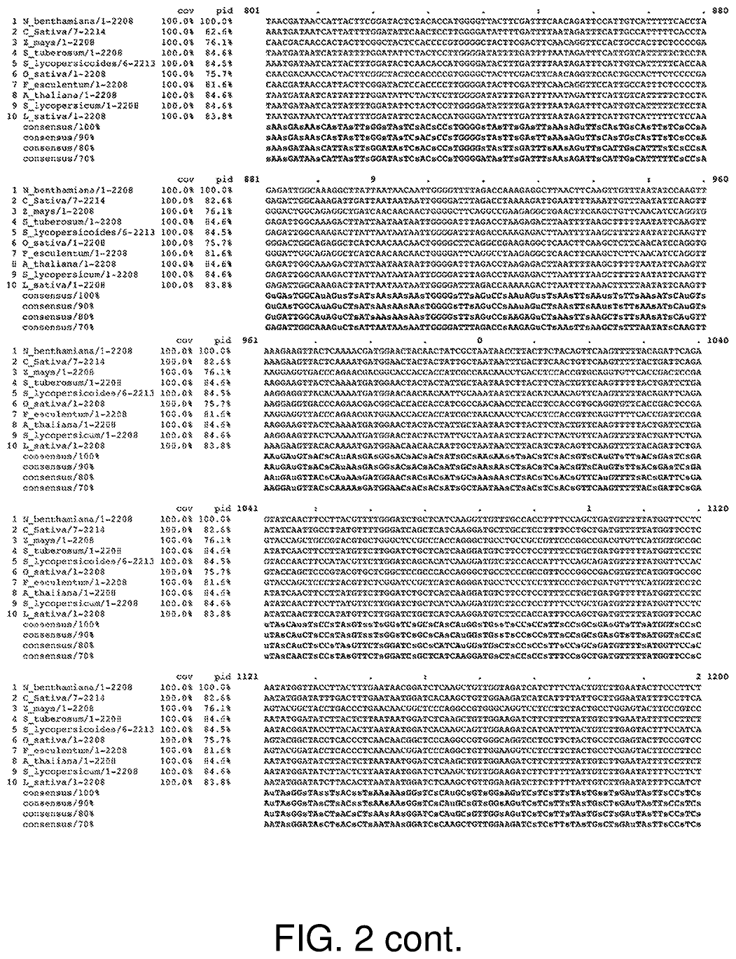

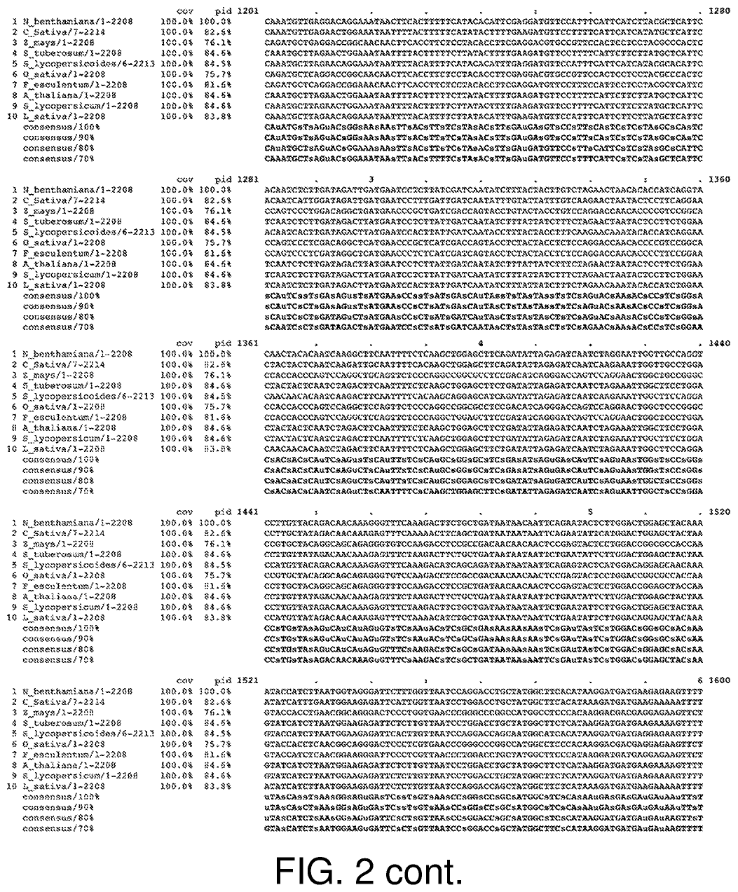

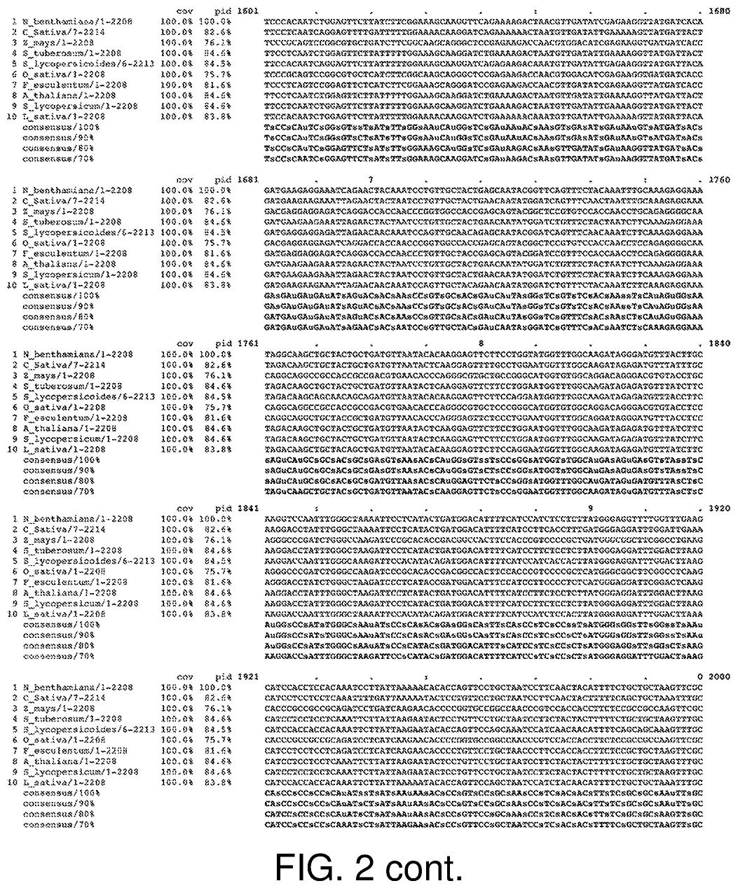

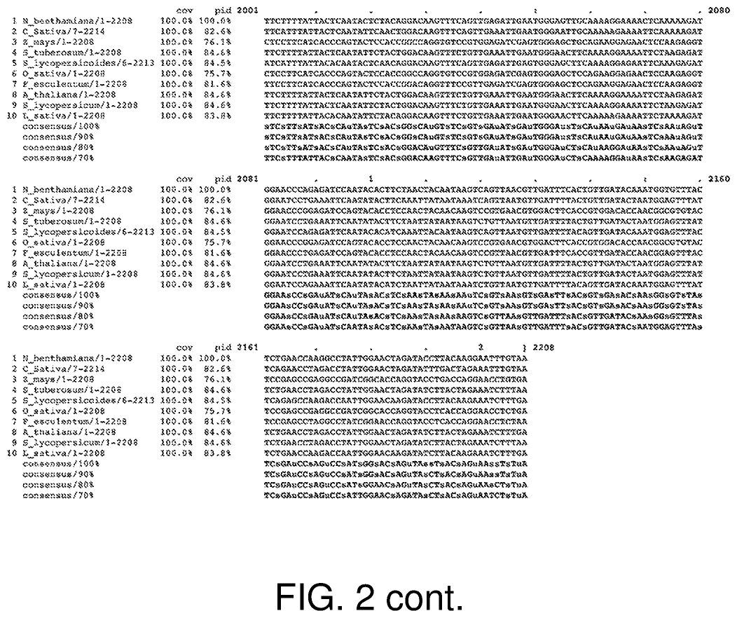

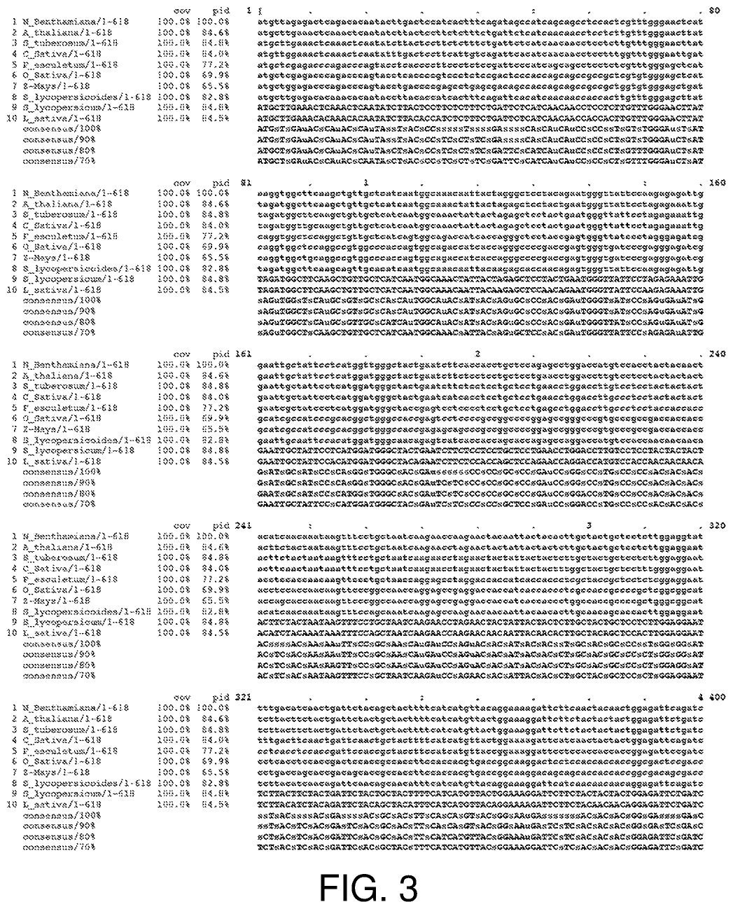

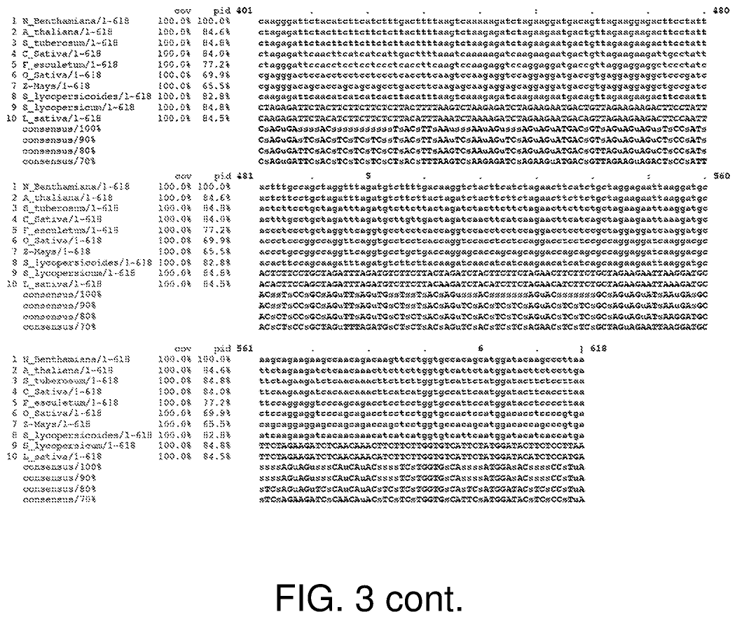

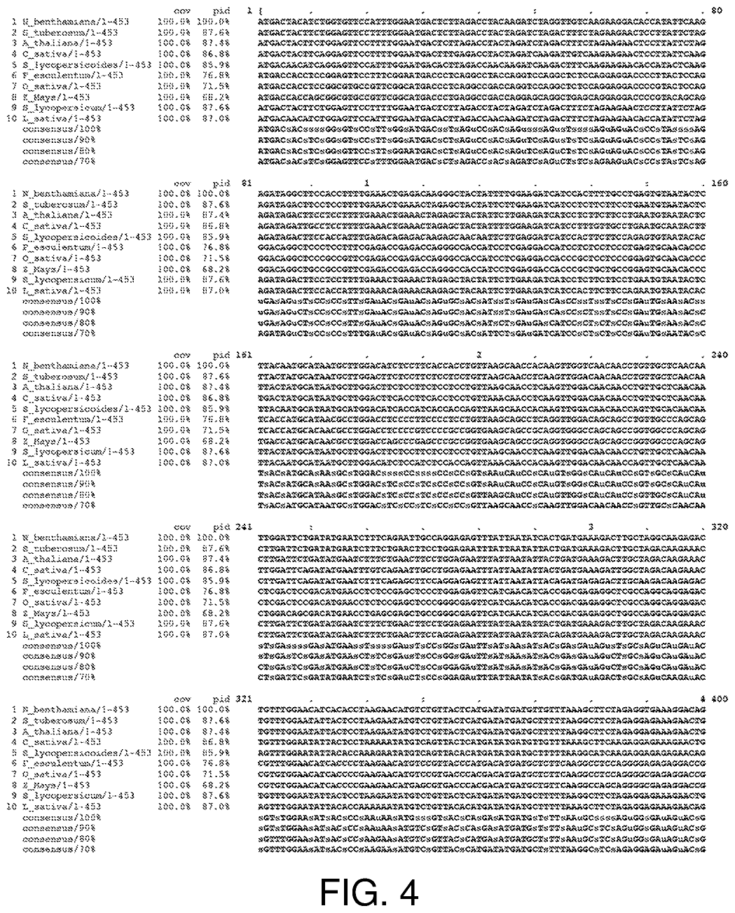

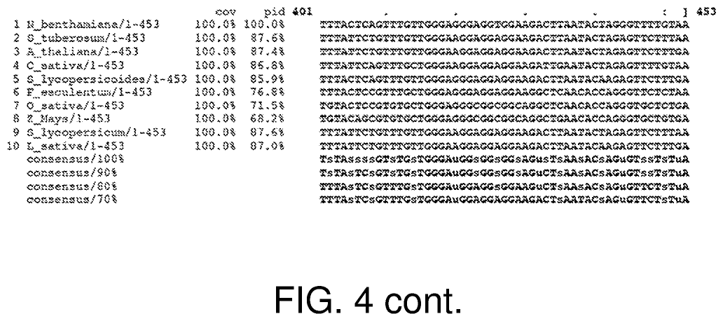

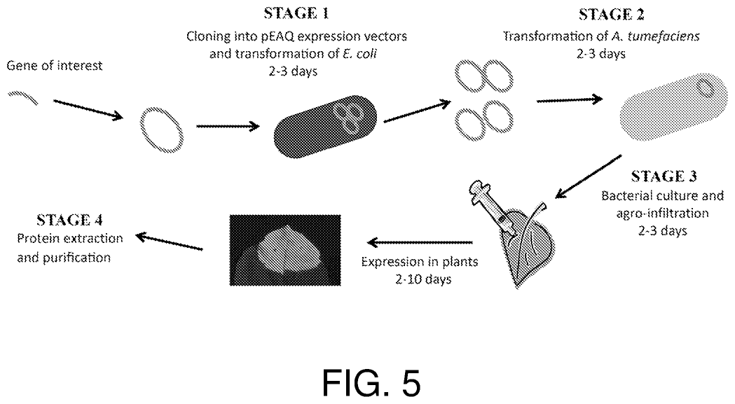

Nucleic acid sequence alignments with N. benthamiana, A. thaliana, S. tuberosum, C. sativa, F. esculentum, O. sativa, Z. mays, S. lycopersicoides, S. lycopersicum, and L. sativa codon optimized cDNA sequences for AAV2 REP (FIG. 1), AAV2 CAP (FIG. 2), AAV2 AAP (FIG. 3), and Ad5 E4orf6 (FIG. 4) are provided.

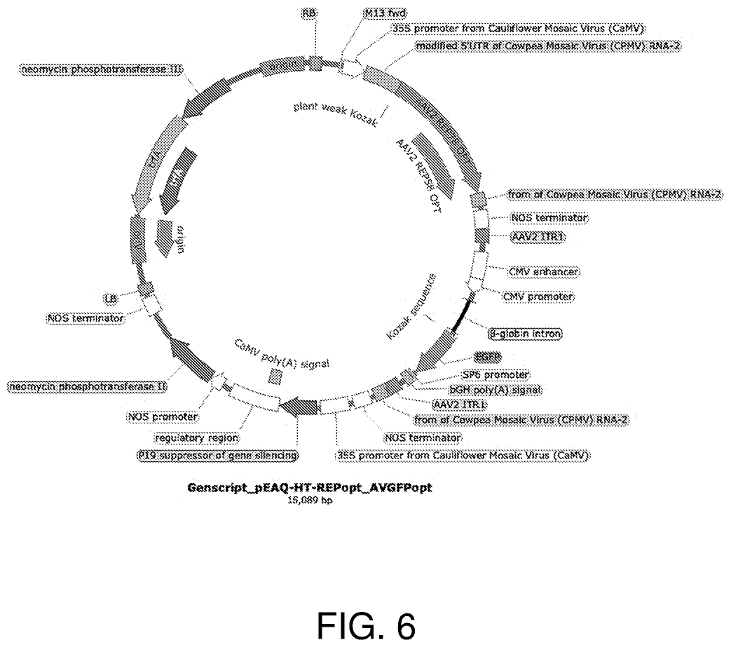

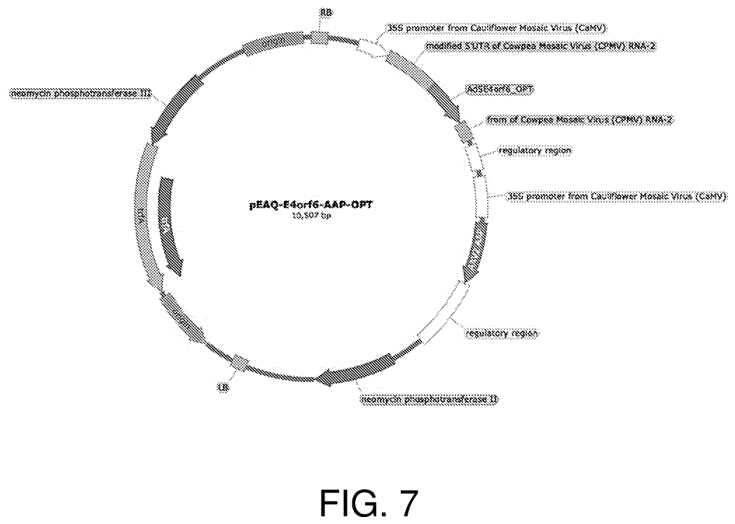

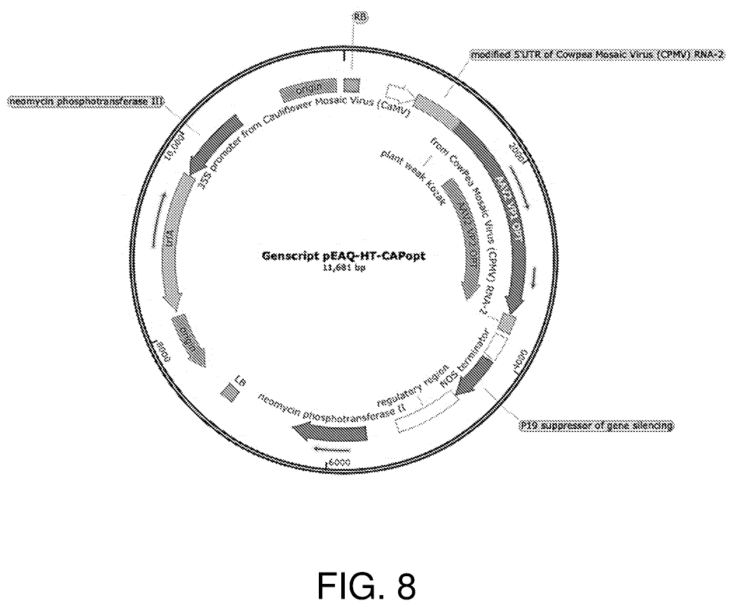

The necessary codon optimized AAV2 and Ad5 sequences were inserted into pEAQ-HT plant infiltration vectors. The codon optimized REP nucleic acid sequence and codon optimized ITR-flanked transgene (SEQ ID NO: 51), comprising EGFP driven by the strong constitutive cytomegalovirus (CMV) mammalian promoter, were inserted into the plasmid pEAQ-HT-REPopt_AVGFPopt (FIG. 6). The codon optimized AAP and E4orf6 nucleic acid sequences were inserted into the plasmid pEAQ-HT-Ad5Orf6-OPT_AAV2-AAP-OPT (FIG. 7). The codon optimized CAP nucleic acid sequence was inserted into the plasmid pEAQ-HT_CAPopt (FIG. 8). Concurrent expression of these three plasmids in a plant cell results in fully assembled AAV2-CMV-EGFP virus particles.

### Example 2: Propagation of N. benthamiana

### Germination Protocol

1. Grodan rockwool cubes (2″×2″×1.5″) were prepared by soaking them in a fertilizer solution of 80 ppm at pH 5.8-6.2 for 5 minutes. One example of fertilizer is VEG+BLOOM RO/Soft (Hydroponic Research) at 0.2-2 g/L supplemented with SuperThrive vitamin solution added at 0.25 mL/L.

2. N. benthamiana seeds were placed on top of each of the prepared rockwool cubes.

3. Seeded cubes were placed into a grow tray and a humidity dome was placed over the tray. The vents were left slightly open to allow air exchange.

4. The tray and dome were placed into a greenhouse. If being germinated in sunlight, a shade-cloth was used over the dome. If being germinated under a grow-light, no shading was needed. Light cycle was set to 16 hours light and 8 hours dark cycles (16L/8D). In greenhouse conditions, supplemental light was added in order to ensure sufficient hours of light are present to keep the tobacco from flowering prematurely.

5. Temperatures were kept between 75-80 degrees Fahrenheit during germination. Temperatures should never drop below 65 degrees Fahrenheit. The root development of the seedling can be seriously impaired when subjected to low temperatures.

6. The surface of rockwool was kept moist at all times. This was achieved by a light misting from a spray bottle. Every other day, each rockwool starting cube was picked up and tested for moisture through touch. If dry, the cube was misted with solution from spray bottle until the cube was wet to the touch. Care was taken not to overwater. Overwatering will impede root development of seedling.

7. When seedlings were kept under optimal conditions, germination was observed within 7-14 days. If both seeds germinate, one was selected and removed so that there was only one plant per cube.

8. The humidity dome was removed once growth is observed.

9. The cubes were kept moist and fed with a spray bottle until roots were observed protruding from the bottom of the cube.

Growth and Manicuring Guidelines

As multiple roots began to protrude from the bottom of the 2″×2″×1.5″ Grodan cubes, they were transferred to a Grodan Delta 4 cube (3″×3″×2.5″). These cubes were prepared in the same manner as outlined in the germination protocol. The plants were grown under same conditions as during germination. The humidity dome was not used. This step typically occurred 7-10 days after seedlings have begun to sprout from the rockwool.

As plants began to transition from the germination to vegetation stage, the apical growth bud was removed. This process is also commonly referred to as topping. This will allow for heavy vegetative leaf growth. Directly after the process of topping, the infiltration protocol was performed.

Heavy sucker growth (axillary bud), apical bud, and perhaps even calyx growth (flower bud) was observed after topping. It is extremely important to remove these growths in order to force the plant to focus growth in the infiltrated leaves, thus providing more biomass in the leaves of interest.

This process was continued on a daily basis for at least 2 weeks or however long as determined through testing is needed in order to allow for expression of the viral capsids inside the leaves.

### Example 3: Infiltration of N. benthamiana with Agrobacterium tumefaciens Containing AAV2-CMV-EGFP Helper Plasmids

Plasmids for the production of AAV2-CMV-EGFP (pEAQ-HT-Ad5Orf6-OPT_AAV2-AAP-OPT, pEAQ-HT_CAPopt, or pEAQ-HT-REPopt_AVGFPopt) were transformed into A. tumefaciens strains AGL1, GV3101 or LBA4404 (Intact Genomics Inc.) via electroporation as detailed in the manufacturer recommendations. Briefly, competent cells were thawed on ice, and DNA to be transformed (1 μL) was added to the pre-chilled tubes on ice. When the cells were thawed, they were added (25 μl) to the chilled DNA on ice and mixed gently by tapping. The cell/DNA mixture (26 μl) was pipetted into a chilled 1 mm electroporation cuvette without introducing bubbles and electroporated (exponential mode, 1800V, 25 FD, 200 ohms). Recovery medium was immediately added (976 L) and electroporated cells in recovery medium were transferred to Eppendorf tubes and incubated at 30° C. for 3 hours with shaking at 200 rpm before plating on to selective medium and culturing for 2 days at 30° C. A. tumefaciens strains transformed with individual helper plasmids were prepared for infiltration using a modified protocol of Sainsbury and Lomonossoff (Plant Physiol. 2008; 148(3):1212-8). Briefly, a single colony of recombinant bacteria was inoculated into liquid LB Lennox or Miller media containing kanamycin (100 mg/L) and rifampicin (50 mg/L). Cultures were incubated overnight at 28° C. with shaking. Bacteria were pelleted by centrifugation (14,000×g for 5 min) and resuspended to an OD₆₀₀=1.0 in optimized infiltration buffer (100 mM MES pH 5.6, 10 mM MgCl2, 300 μM acetosyringone, 5 μM α-lipoic acid, 0.002% Pluronic F-68). Cultures were then incubated for 2-4 hours at room temperature with gentle rocking. For small scale experiments, bacteria were delivered into the underside of leaves of 3-6 week plantlets using a blunt tipped plastic syringe and applying gentle pressure. For whole plant infiltration, 3-6 week old plantlets were completely submerged in 1-3 L of infiltration buffer inside a vacuum desiccator unit containing Agrobacterium strains transformed with the helper plasmids, generated above. The desiccator unit was sealed, and the plantlets were infiltrated by applying a vacuum of 100 mBar for 1 min and then releasing vacuum. This was repeated two times. In both cases, recombinant bacterial strains containing the individual helper plasmids were mixed at a 1:1:1 ratio (pEAQ-HT-Ad5Orf6-OPT_AAV2-AAP-OPT:pEAQ-HT_CAPopt:pEAQ-HT-REPopt_AVGFPopt) immediately prior to infiltration. Whole plants were subject to heat shock 2 days post infiltration (37° C. for 30 min) to increase transient helper protein expression.

### Example 4: Purification of AAV2-CMV-EGFP from N. benthamiana Leaf Tissue

Agroinfiltrated N. benthamiana leaves were removed as close to the base of the plant as possible using sterilized garden shears. Once removed, leaves were placed in a chlorine dioxide fumigation chamber to sanitize for 10 minutes, followed by 3 washes in sterile de-ionized distilled water. Total leaf protein from the sanitized leaves was extracted by homogenization with extraction buffer (25 mM sodium phosphate, 100 mM NaCl, 50 mM sodium ascorbate, 2 mM PMSF, pH 5.75) with a Hamilton blender following the manufacturer's instruction. The crude plant extract was clarified by centrifugation at 14,000×g for 10 min at 4° C.

After 1 hour of incubation at 4° C., the homogenates were centrifuged at 6,000×g for 30 minutes at 4° C. to remove leaf debris and the abundant plant photosynthetic enzyme ribulose 1,5-bisphosphate carboxylase-oxygenase (RuBisCO). The supernatant was then incubated at 4° C. for 24 hours and centrifuged for 30 minutes at 6,000×g at 4° C. to further remove RuBisCO that precipitated during incubation. This process was repeated for a total of 3 times to completely remove residual RuBisCO. The supernatant was then filtered with a 0.22 μM filter (Millipore). The clarified supernatant was then concentrated using ultrafiltration/diafiltration (UF/DF) with a 100 kDa polyethersulfone tangential (PES TFF) membrane (Pall Corporation) to remove any residual plant-derived small molecules whilst retaining the recombinant AAV2 particles. Pre-filtered clarified supernatant containing crude rAAV2 particles was then further purified by sequential affinity and ion exchange chromatography. Briefly, the clarified cell lysate containing the rAAV vectors was loaded onto an AVB Sepharose HP column (GE Life Sciences). Columns with bound rAAV particles were washed with wash buffer (20 mM Tris HCl, 0.5 M NaCl, pH 8.0) to remove all unbound proteins and contaminants as measured by absorbance at A₂₆₀ and A₂₈₀. The bound rAAV was then eluted with low-pH buffer. The eluted rAAV solution was immediately neutralized by adding 1 M Tris-HCl (pH 8.7) at 1/10 of the fraction volume directly into the fraction collection tube prior to elution. Following AVB affinity purification, the AAV vector was further purified using anion exchange chromatography by binding and elution from a POROS 50HQ (ThermoFisher) anion exchange column to separate empty from full (genome containing) particles. Bound AAV capsids were eluted with increasing conductivity in the presence of a 10-mM to 300-mM Tris-acetate gradient (pH 8), and sequential fractions enriched for full rAAV2 particles were collected, pooled and then diafiltered into formulation buffer (180 mM NaCl, 10 mM Sodium phosphate, 0.001% Pluronic F-68) by spinning at 3,000×g through a Vivaspin 15R 30 kD diafiltration column. This was repeated 3 times with addition of formulation buffer each time. Purified and concentrated rAAV2-CMV-EGFP viral vectors were then aliquoted into low protein binding tubes and stored at −80° C.

### Example 5: Titration of AAV2-CMV-EGFP Purified from Leaf Tissue Using qPCR

Purified rAAV-CMV-EGFP viral particles (2 μL) and AAV2-CMV-EGFP reference control vector with a known genomic titer (2 μL) (ATCC #VR-1616) were denatured using 50 μL of AAV PCR alkaline digestion buffer (25 mM NaOH, 0.2 mM EDTA) for 10 min at 100° C. Samples were then cooled on ice and neutralized by addition of 50 μL of neutralization buffer (40 mM Tris-HCl, pH 5.0). For each sample, quantitative PCR reactions were set up in triplicate using SYBR Green qPCR Master Mix (Sigma) and primers designed to amplify the EGFP transgene by the conserved ITR sequences (forward: 5′-GGAACCCCTAGTGATGGAGTT-3′ (SEQ ID NO: 52), reverse: 5′-CGGCCTCAGTGAGCGA-3 (SEQ ID NO: 53). AAV2 reference standard were prepared identically using the same master mix and a standard curve was generated by making a log dilution series of the reference vector ranging from 1×10⁹ viral genomes per mL (vg/ml) to 1×10⁴ vg/ml. Titers of plant-produced AAV2-CMV-EGFP are calculated by fitting relative cycle quantification (C_{q}) values to the reference standard curve.

### Example 6: qPCR Quantification of Plant-Produced AAV2-CMV-EGFP

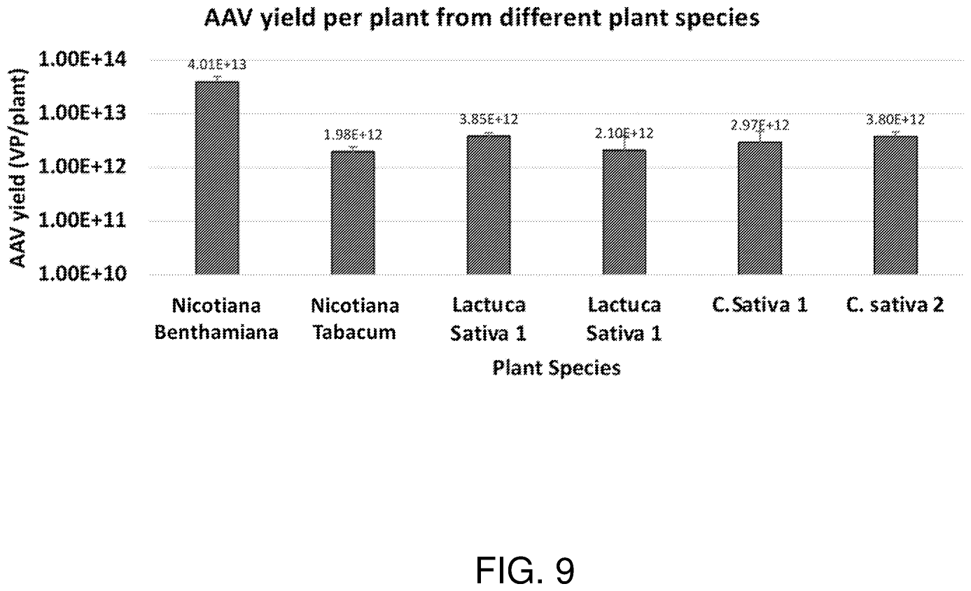

AAV2-CMV-EGPF vector was produced by transient vacuum mediated infiltration of plant codon optimized AAV2 producer plasmids transformed into Agrobacterium. Plants tested were N. benthamiana, N. tabacum, L. sativa, and C. sativa. The L. sativa and C. sativa samples were performed in duplicate. Five days post infiltration plant leaves were harvested, extracted, and AAV2-CMV-EGFP particles were purified using low pH precipitation of plant proteins followed by centrifugation, filtration, and concentration as described herein. Purified AAV2-CMV-EGFP vector preparations were treated with DNAse I to remove any non-encapsidated DNA and batches were titrated using quantitative real time PCR with primers targeting the AAV2 specific ITRs (as described in Example 5). Relative genomic yields per plant were calculated by comparison to a standard curve of known amounts of linearized AAV2-CMV-EGFP plasmid. A range of 10¹² to 10¹⁴ viral genomes per plant was quantified, with N. benthamiana resulting in the greatest relative yield of viral genomes (FIG. 9)

### Example 7: Assessing Protein Content and Purity of AAV2-CMV-EGFP Produced in Leaf Tissue

Purity of the purified and concentrated rAAV particles was assessed by SDS-PAGE with silver stain or other compatible stain. Two volumes of the purified rAAV preparation (e.g. 2 μL and 6 μL) were directly denatured in reducing tris-glycine SDS sample buffer to a final volume of 15 μL and heated to 95° C. for 5 minutes. A volume range (e.g. 0.5, 1, 2, 3, and 4 μL) of an AAV2 reference standard (ATCC) was processed in the same manner. Equivalent volumes of samples were loaded onto an SDS-PAGE gel, and run at 50-200 V for 1-3 hours or until the dye front had run off the gel. The gel was processed for silver staining according to manufacturer's instructions or protocols known in the art. A pure rAAV sample will result in only three bands corresponding to VP1 (87 kDa), VP2 (73 kDa), and VP3 (62 kDa).

Purity can also be assessed by other techniques known in the art, such as capillary electrophoresis or mass spectrometry.

### Example 8: Detection of AAV2 VP1/2/3 Capsid Proteins by SDS-PAGE from Leaf Lysates from AAV2-CMV-EGFP Producing Plants

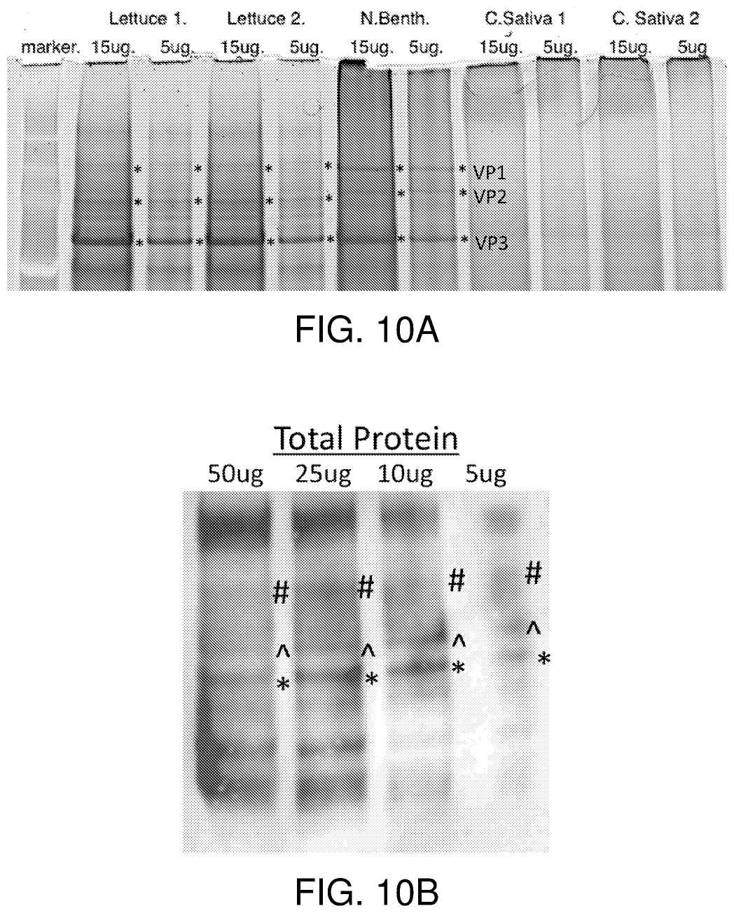

AAV2-CMV-EGFP vectors were produced in N. benthamiana, L. sativa (2 replicates), and C. sativa (2 replicates) by vacuum mediated infiltration of plant codon optimized AAV2 producer plasmids transformed into Agrobacterium. Five days post infiltration, plant leaves were harvested and lysates were produced using low pH precipitation of abundant plant proteins followed by centrifugation, 0.45 μm filtration, and concentration as described herein. Total protein in leaf lysates was quantified using a BCA assay, and different amounts of total protein (5 μg and 15 μg) were loaded onto a 4-12% Bis-Tris SDS-PAGE gel and run for 1 hour at 190 mV. Protein was detected using the Oriole fluorescent protein stain and visualized on a BioRad gel imager. Robust bands corresponding to VP1, VP2, and VP3 protein were detected in the N. benthamiana and L. sativa leaf lysates (FIG. 10A).

Different amounts of total protein (5 μg, 10 μg, 25 μg, 50 μg) from N. benthamiana leaf lysates after purification were loaded onto a 4-12% Bis-Tris SDS-PAGE gel and run for 1 hour at 190 mV. Proteins were transferred onto a nitrocellulose membrane and Western blotting was performed to detect AAV2 VP1, VP2, and VP3 capsid proteins using an anti-AAV2 VP monoclonal primary antibody and an anti-mouse HRP secondary antibody (FIG. 10B).

### Example 9: Infection of Tissue Culture Cells with AAV2-CMV-EGFP Purified from Leaf Tissue

HEK 293T cells (ATCC #CRL-11268) were plated at a density of 5×10⁴ cells per well into a 12-well culture plate in 1 mL of growth medium per well (DMEM High glucose, 1× GlutaMAX (Corning), 10% FBS, 1% Penicillin-Streptomycin). 6-8 hours after plating, individual wells were infected with plant-produced rAAV2-CMV-EGFP at a multiplicity of infection (MOI) ranging from 500 to 5000 viral genomes (vg) per cell. Infected cells were incubated at 37° C., 5% CO₂ for 36 hours and then infectivity per well was assessed using an inverted fluorescent microscope with excitation and emission filters suitable for EGFP.

### Example 10: EGFP Expression in HEK293T Cells Treated with Plant Produced AAV2-CMV-EGFP

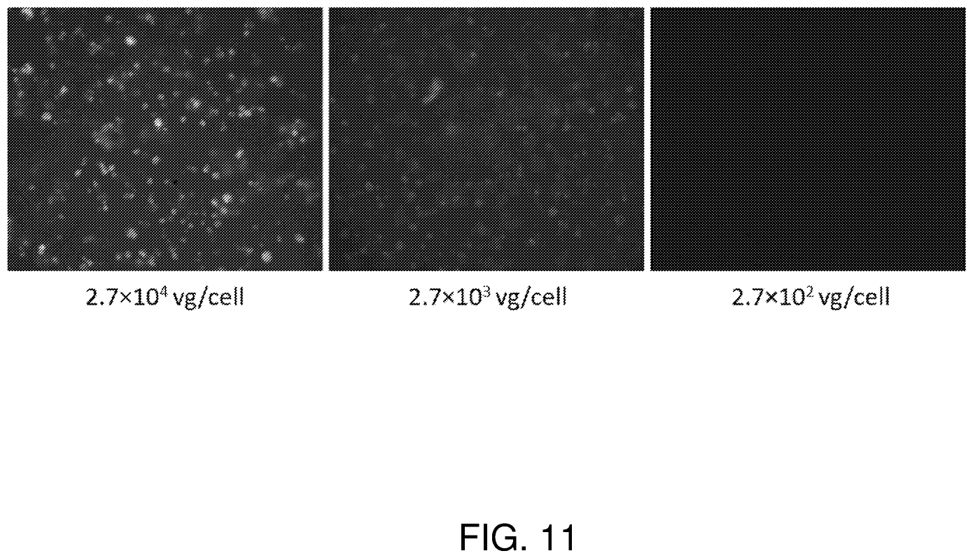

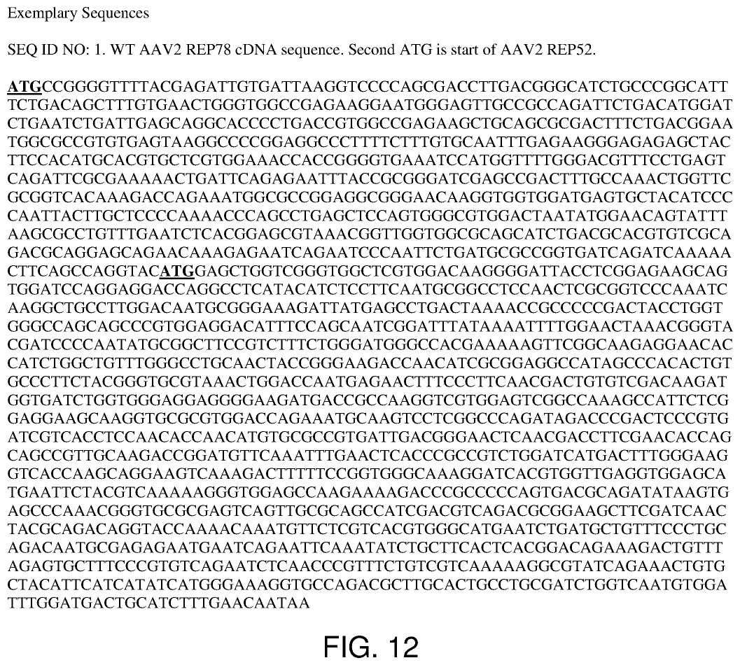

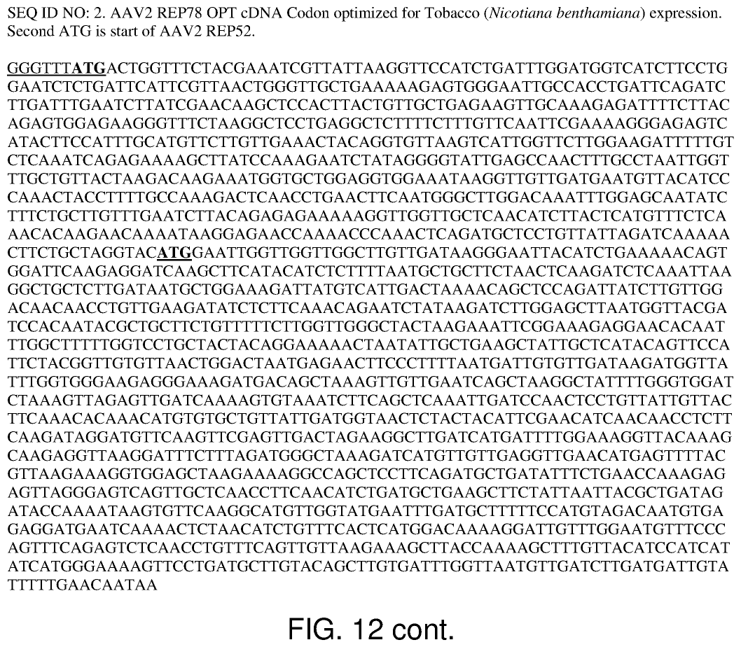

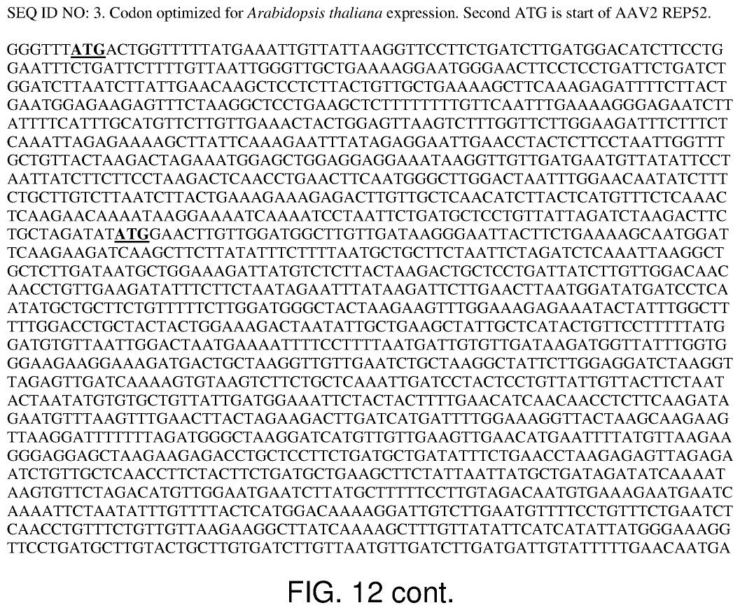

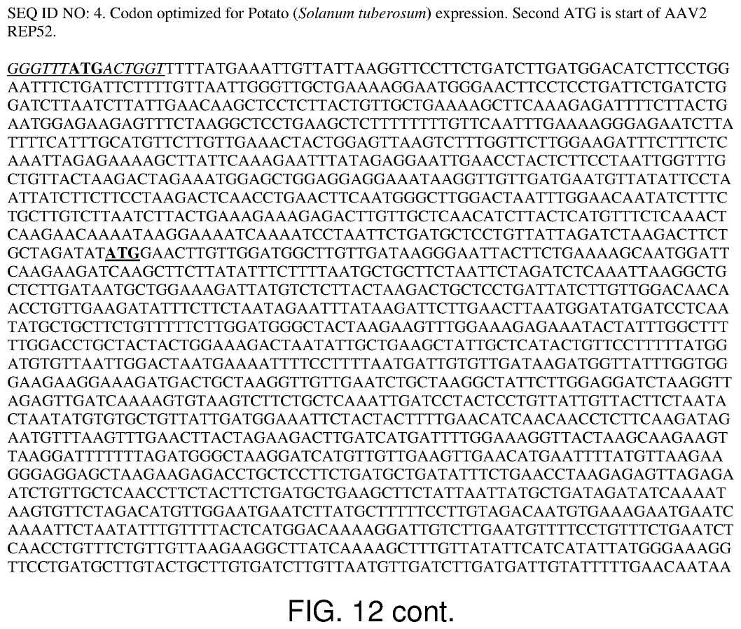

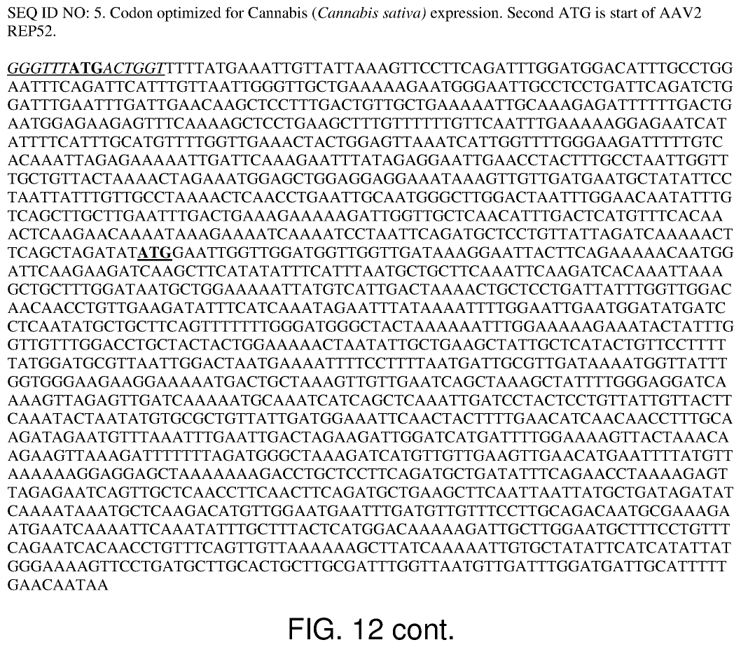

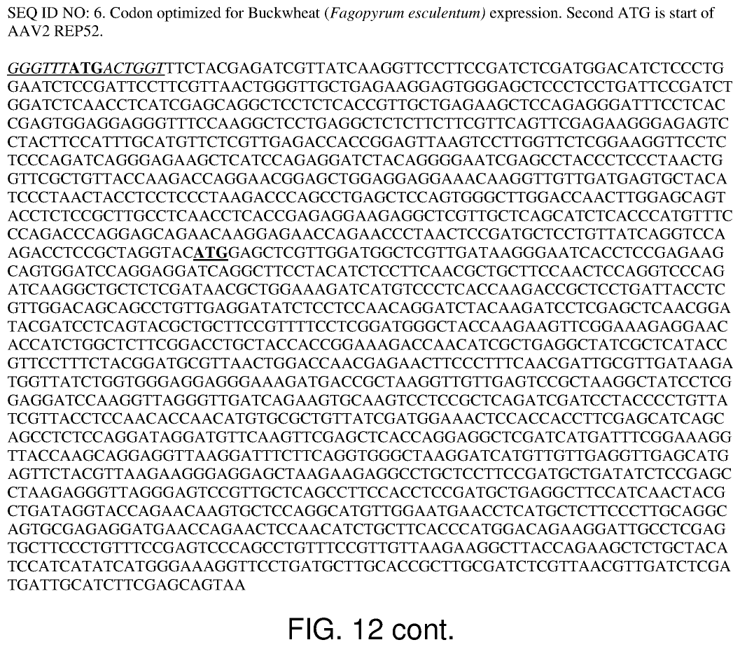

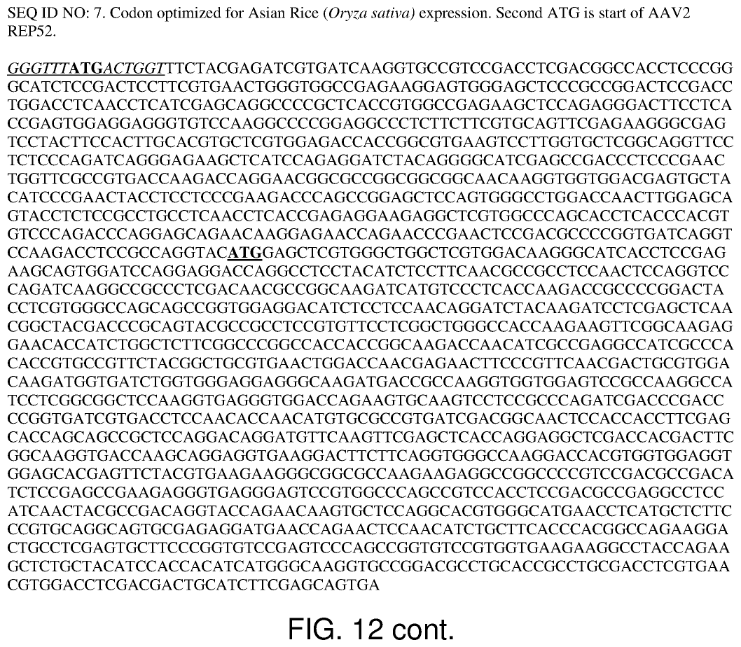

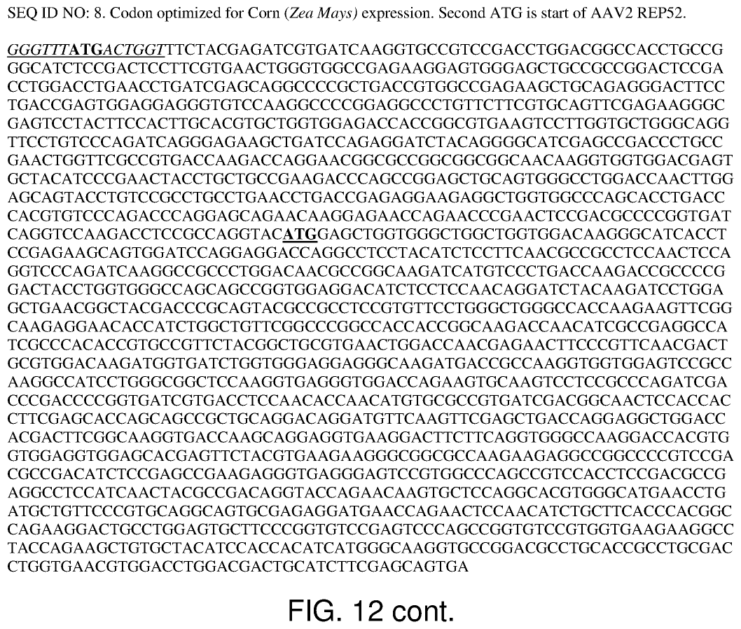

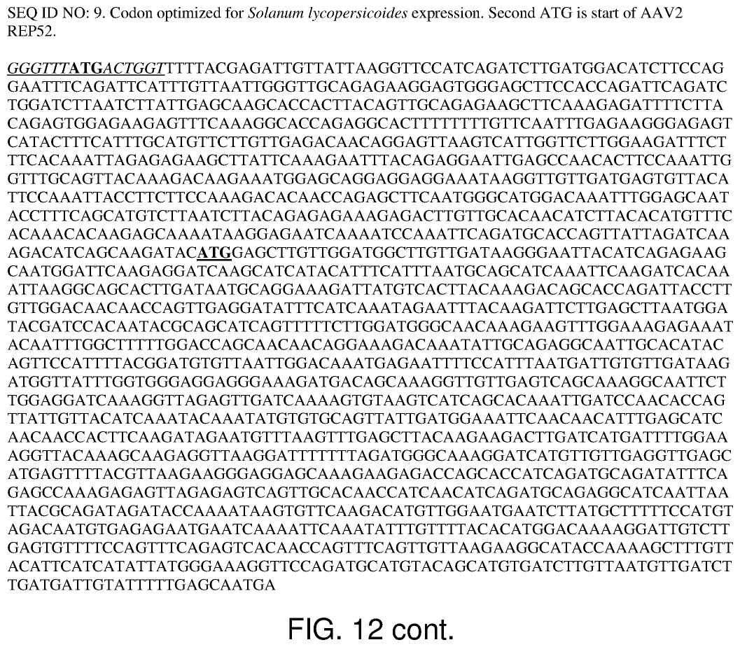

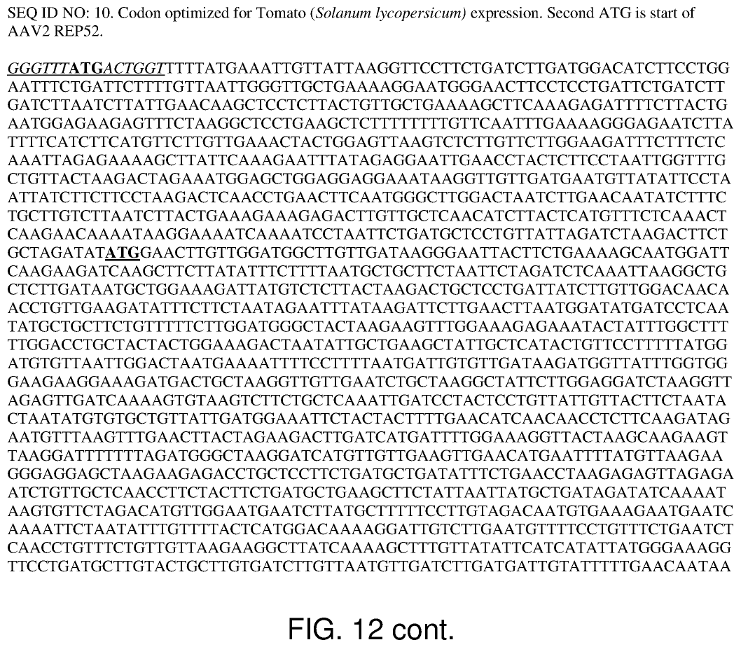

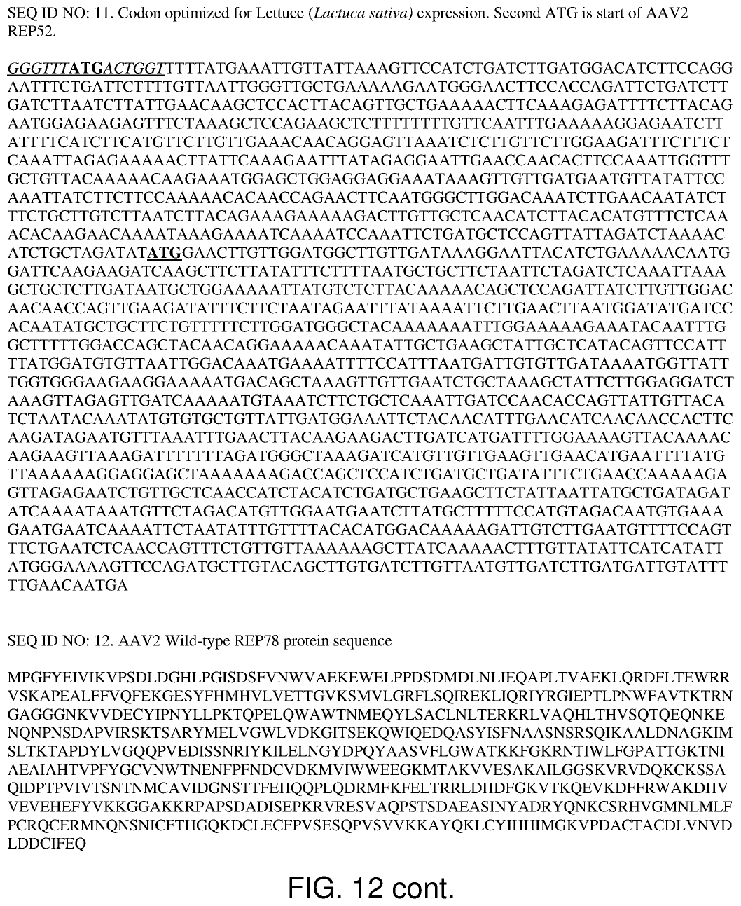

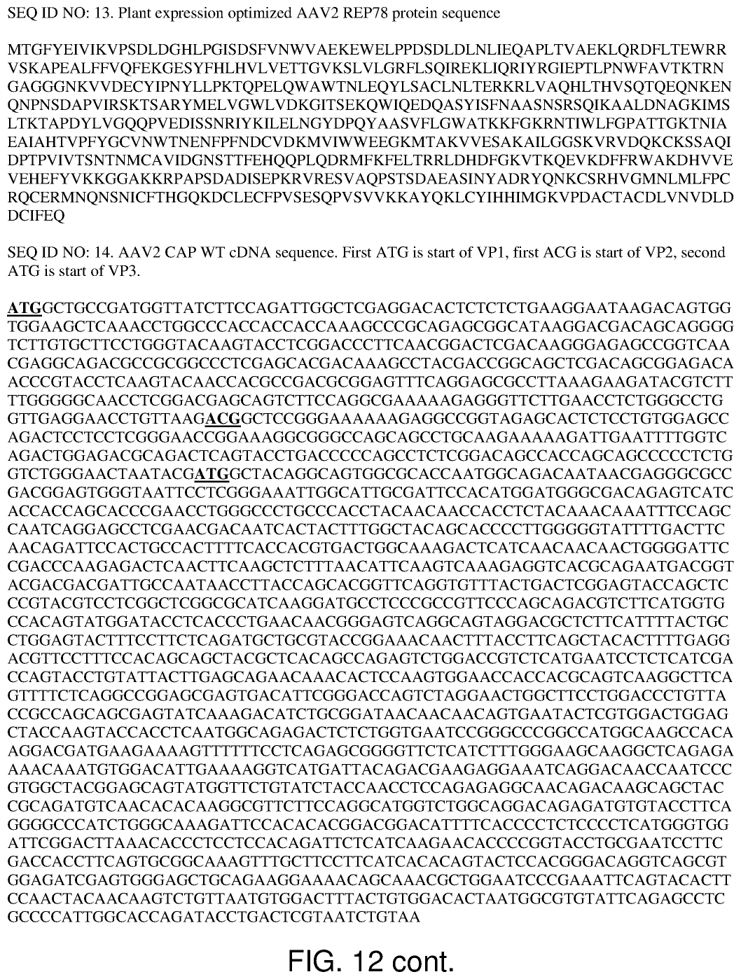

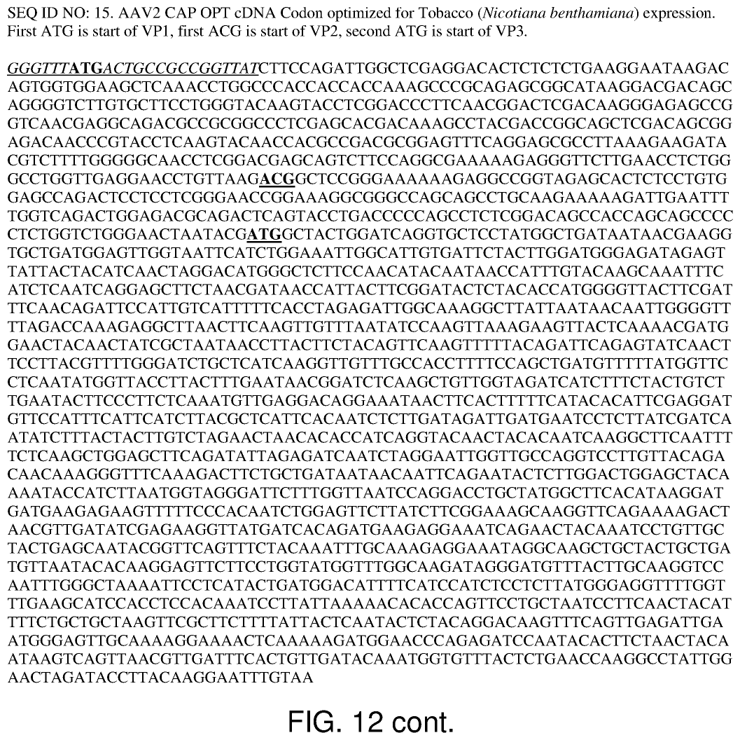

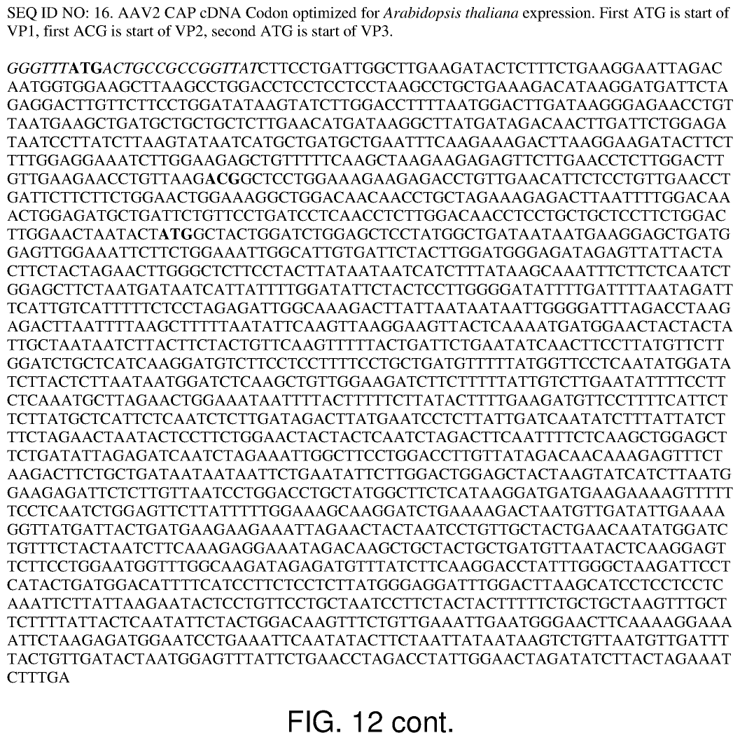

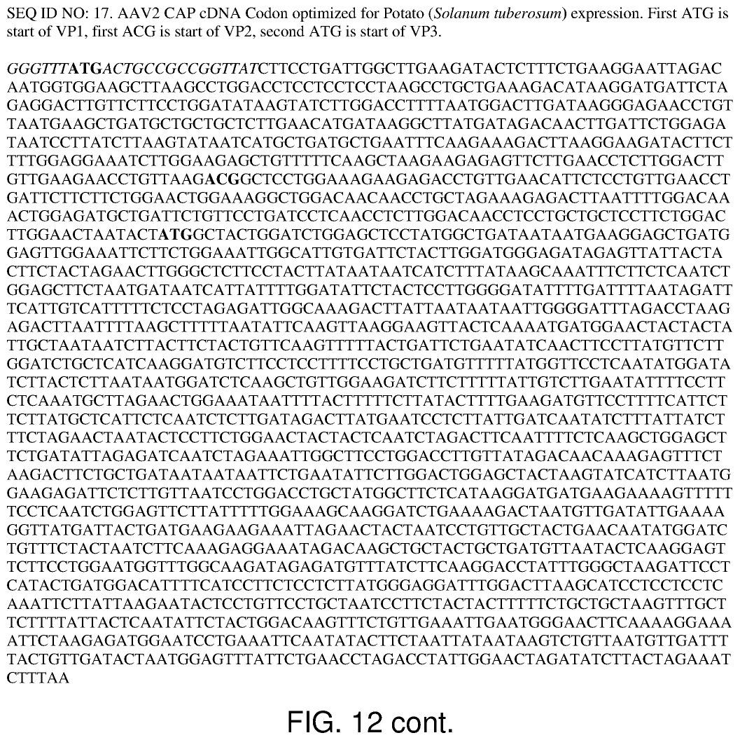

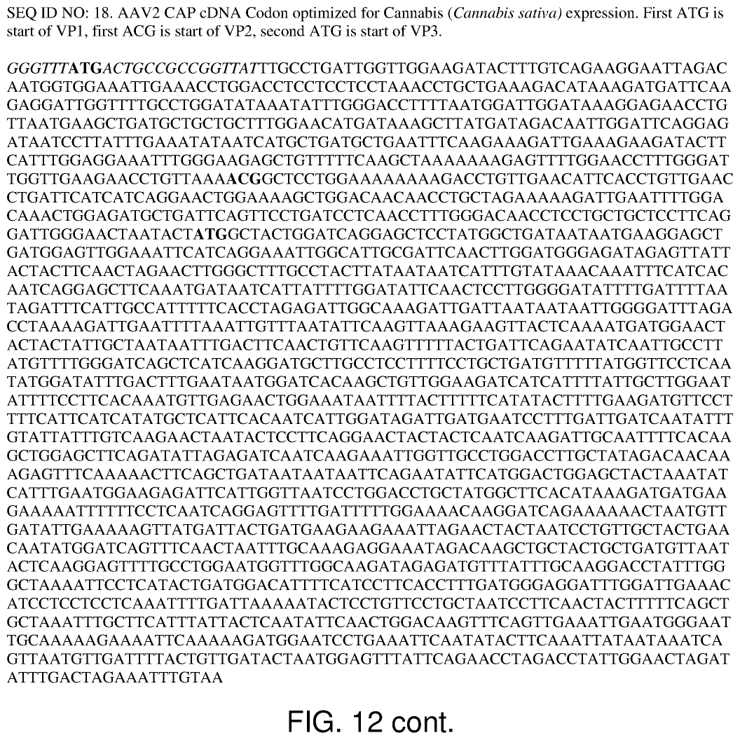

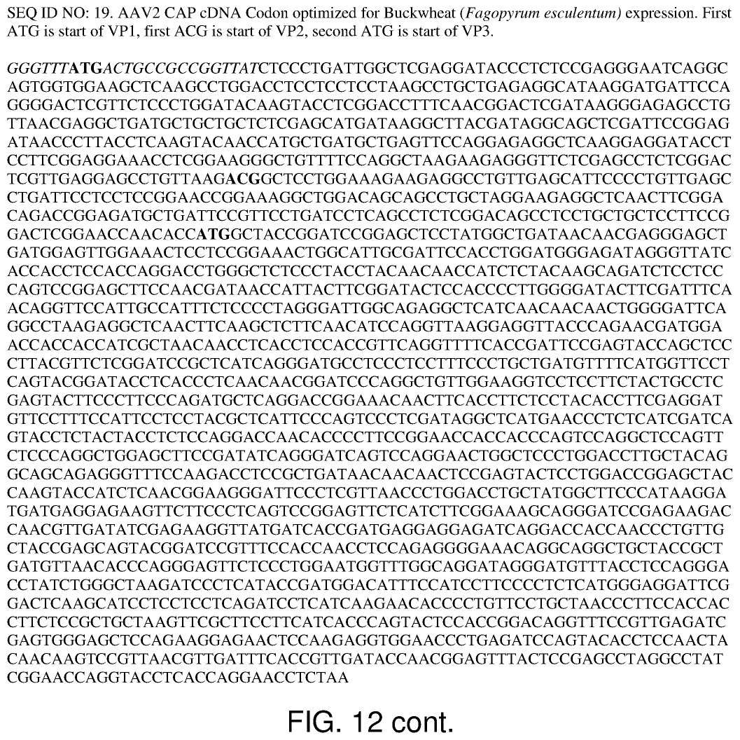

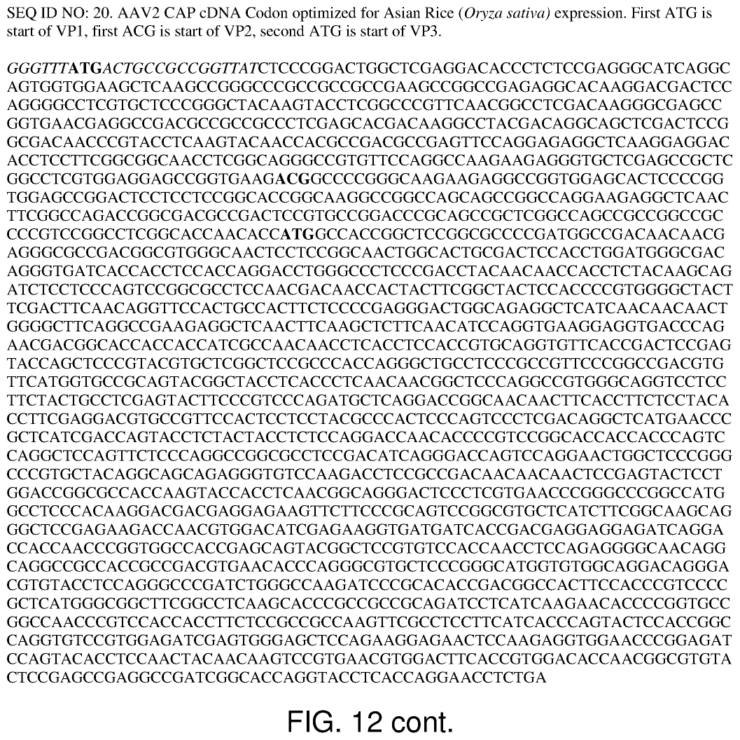

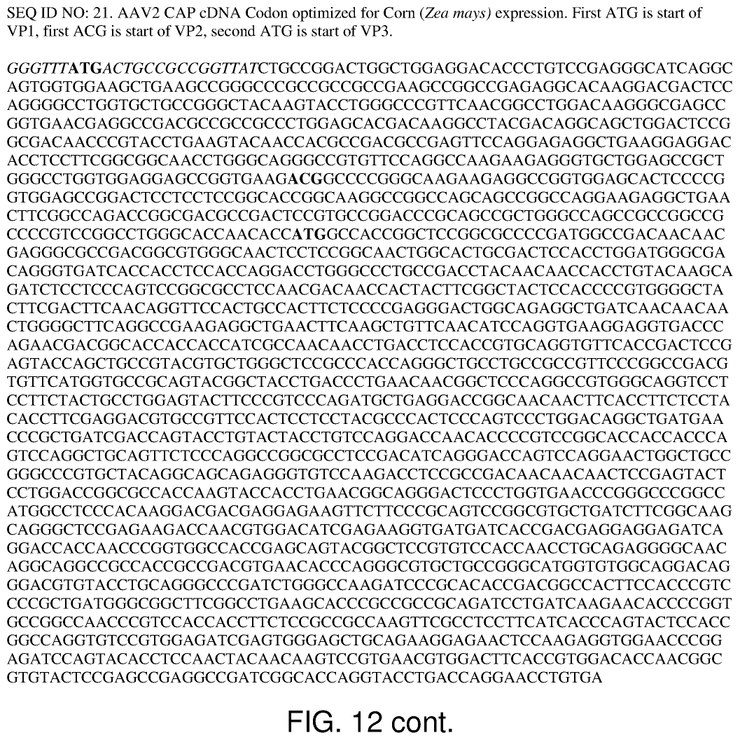

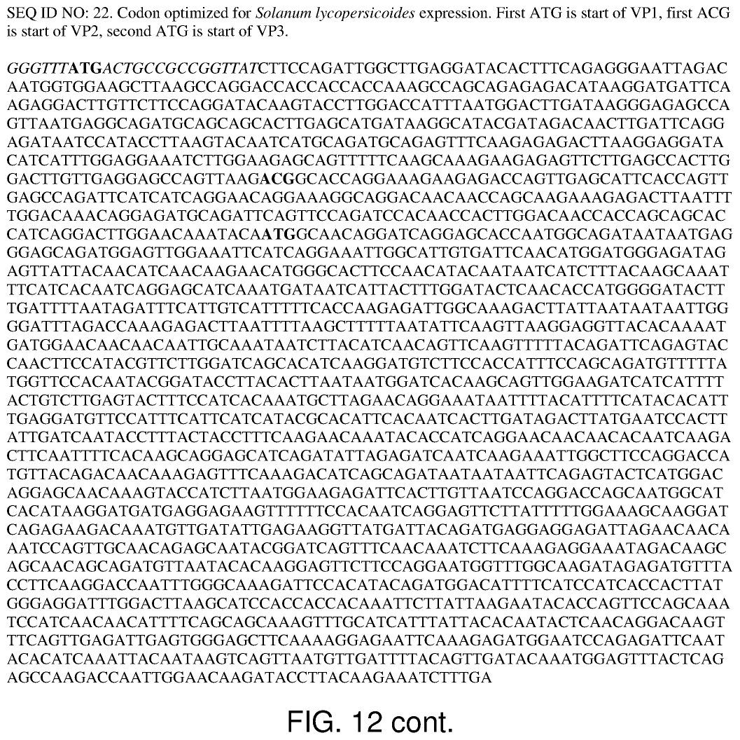

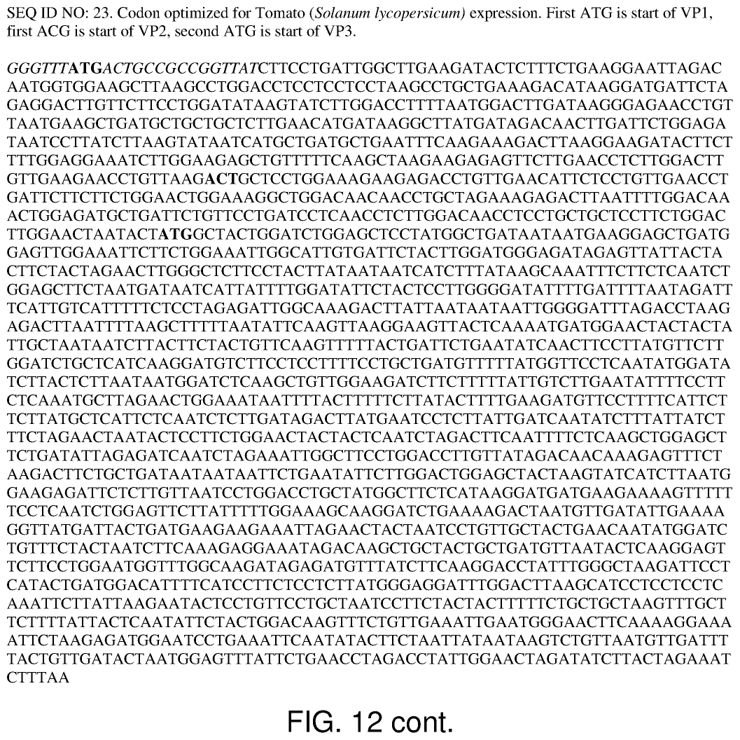

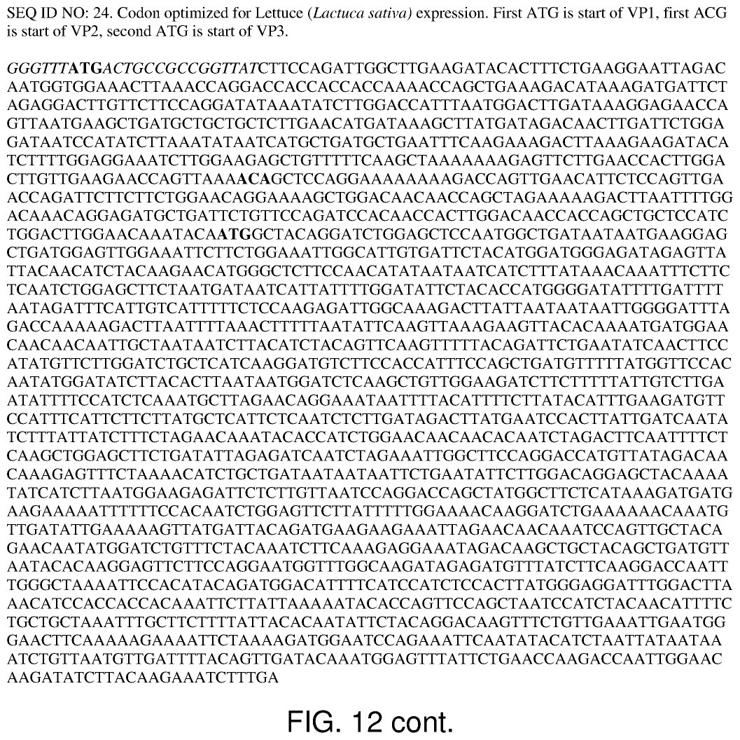

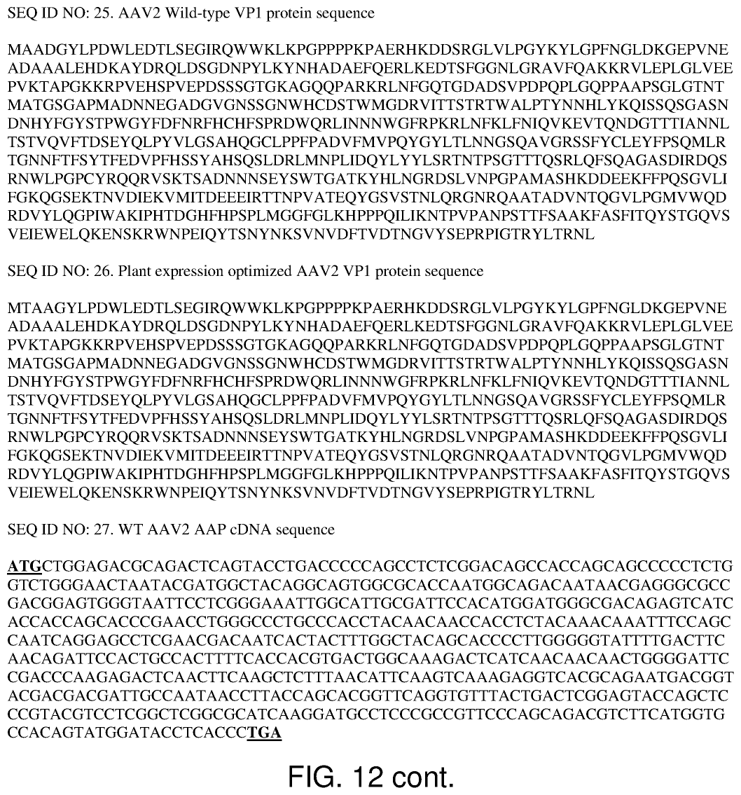

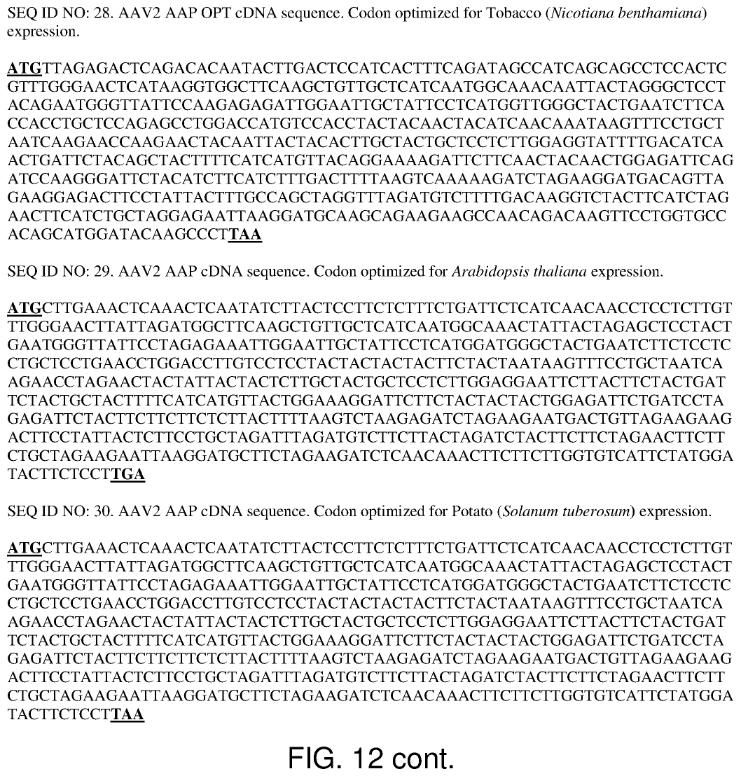

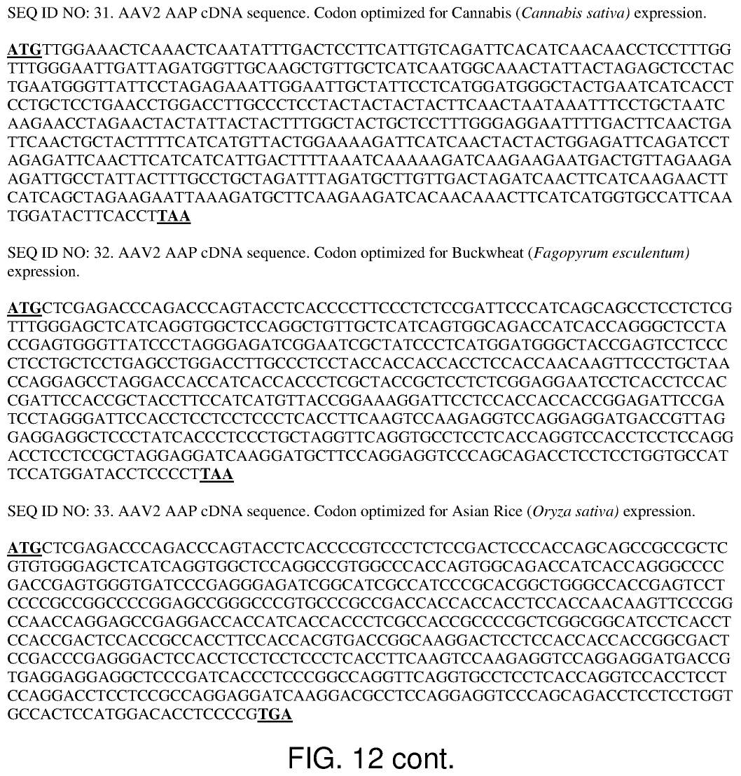

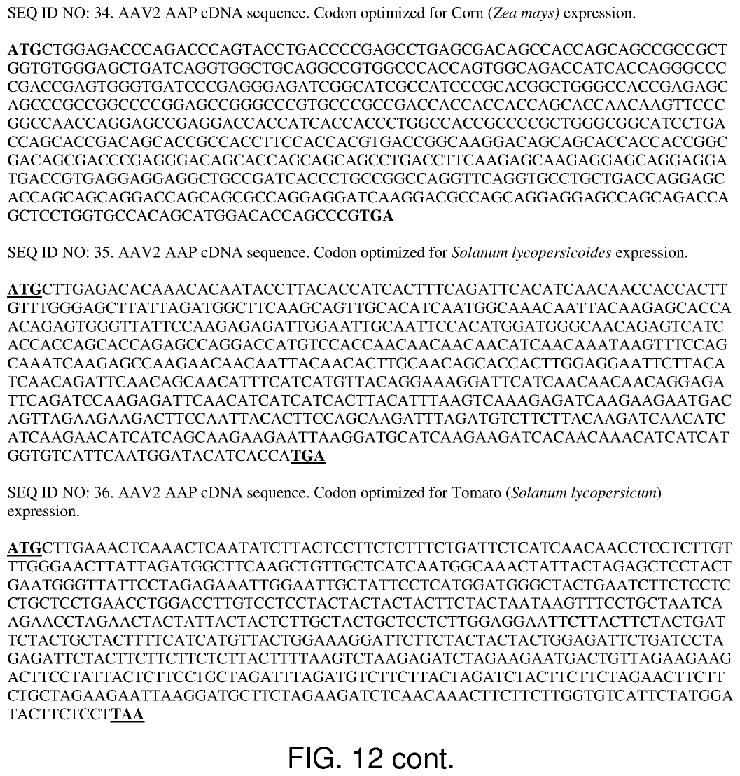

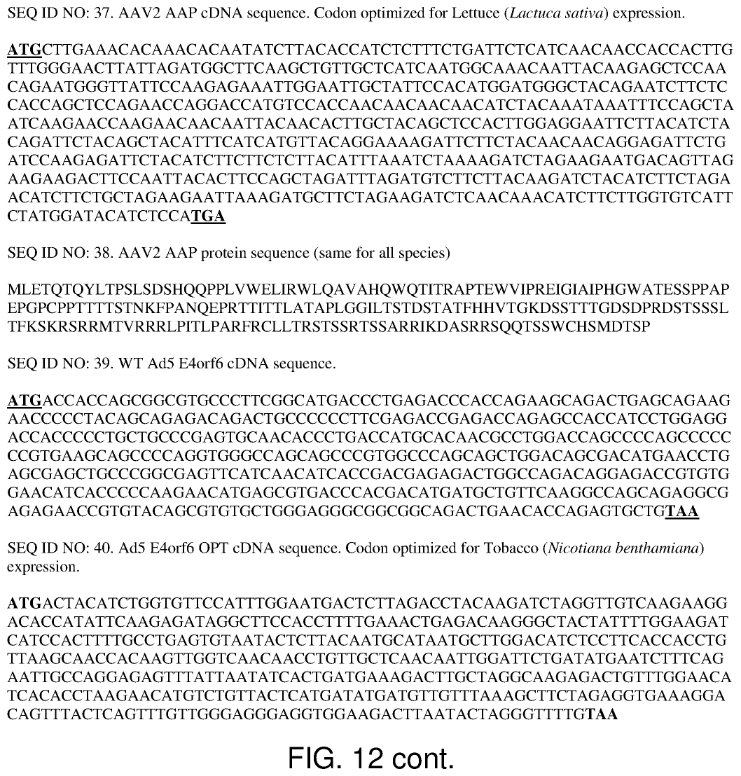

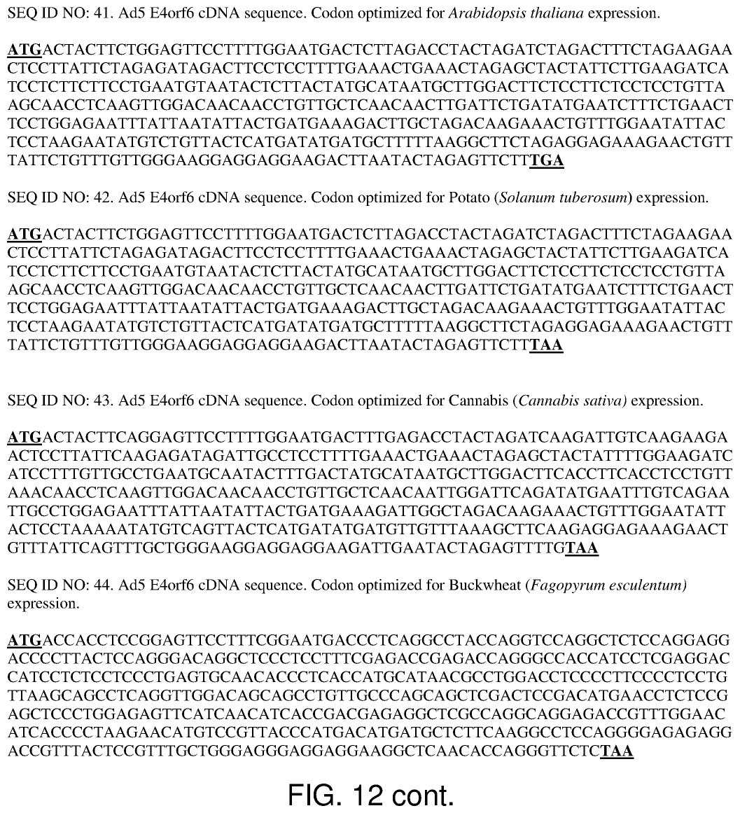

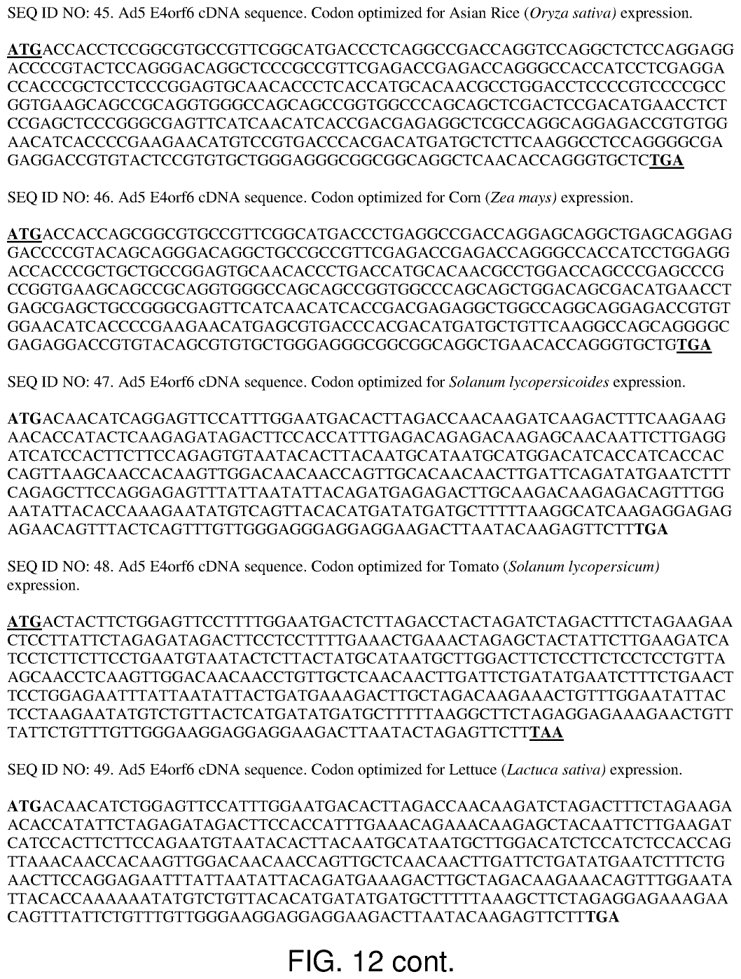

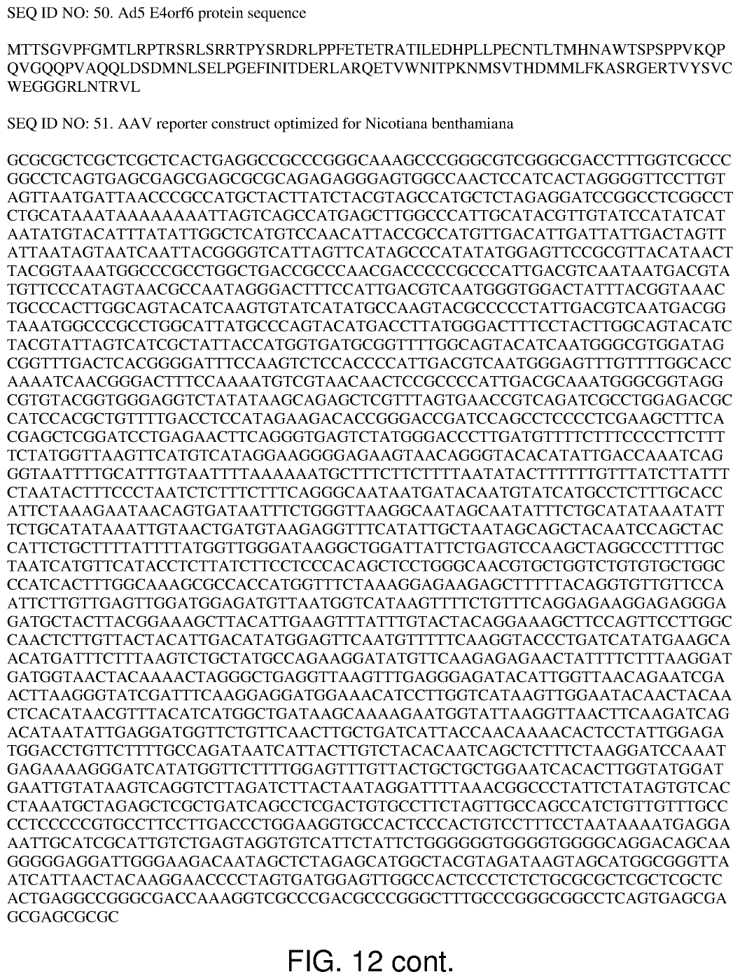

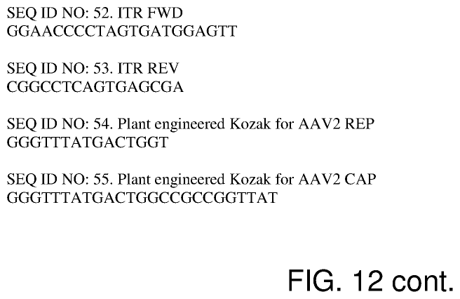

AAV2-CMV-EGFP vectors were produced in N. tabacum plants by transient vacuum mediated infiltration of plant codon optimized AAV2 producer plasmids transformed into Agrobacterium. Five days post infiltration, plant leaves were harvested, extracted, and AAV2-CMV-EGFP particles were purified using low pH precipitation of plant proteins followed by centrifugation, filtration, and concentration as described herein. Purified and titrated AAV2-CMV-EGFP vector at specific multiplicities of infection (2.7×10⁴, 2.7×10³, or 2.7×10² viral genomes per HEK293T cell) were added directly to HEK293T cells grown in 4 chamber slide flasks. Cells were images for native EGFP expression at 4 days post infection. Positive, MOI-dependent EGFP expression in the HEK293T cells was observed by fluorescence microscopy (FIG. 11).

### Example 11: Using Purified AAV2 Particles for Gene Therapy

The recombinant AAV2 viral particles produced in the preceding examples are intact and infective. These particles can be used for gene therapy purposes or other therapeutic purposes. Particles can be used for ex vivo and in vivo treatments or applications. Particles can be administered enterally, parenterally, orally, sublingually, buccally, intranasally, intraocularly, intraaurally, epidurally, epicutaneously, intra-arterially, intravenously, intraportally, intra-articularly, intramuscularly, intradermally, peritoneally, subcutaneously, or directly to an organ, tissue, cancer, or tumor. Particles can also be administered to isolated cells from a patient or individual, such as T cells, Natural Killer cells, B cells, macrophages, lymphocytes, stem cells, bone marrow cells, or hematopoietic stem cells. Particles purified from plants offer improved safety profiles, yield, and efficacy over viral particles purified by other methods, such as from mammalian cell culture or insect cell culture.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A nucleic acid molecule comprising a sequence that encodes an AAV2 REP protein, wherein the sequence has at least 90%, 91%, 92%, 93%, 94%, 95%, 96%, 97%, 98%, 99%, or 100% sequence identity to SEQ ID NO: 2-11.

2-57. (canceled)
